SEQUENCE LISTING (34 sequences)

SEQ ID NO: 1 - protein, 524 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHL

SEQ ID NO: 2 - protein, 133 aa, Homo sapiens [sequence omitted]

SEQ ID NO: 3 - protein, 551 aa, Artificial Sequence
  Description of Artificial Sequence Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - DNA, 1656 bp, Artificial Sequence
  Description of Artificial Sequence Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggacaccattccgtggctcggccacctcctcgtgggtctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 5 - protein, 462 aa, Artificial Sequence
  Description of Artificial Sequence Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPANNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 6 - DNA, 1389 bp, Artificial Sequence
  Description of Artificial Sequence Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 7 - protein, 498 aa, Artificial Sequence
  Description of Artificial Sequence Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 8 - DNA, 1496 bp, Artificial Sequence
  Description of Artificial Sequence Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcacagagacaaggccctctgatcccaccctgggggtggccaggcaacacccttgttgctgtgtccatctttctcctgctgactggcccgacctacctcctgttcaagctgtcgcccagggtgaagagaatcttctaccagaacgtgccctctccagcgatgttcttccagcccctctacagtgtacacaatgggaacttccagacttggatgggggcccacggggccggtgtgctgttgagccaggactgtgctggcaccccacagggagccttggagccctgcgtccaggaggccactgcactgctcacttgtggcccagcgcgtccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattcta

SEQ ID NO: 9 - protein, 545 aa, Artificial Sequence
  Description of Artificial Sequence Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPAEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 10 - DNA, 1638 bp, Artificial Sequence
  Description of Artificial Sequence Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagaggagttaaaggaaggctggaaccctcacctgctgcttctcctcctgcttgtcatagtcttcattcctgccttctggagcctgaagacccatccattgtggaggctatggaagaagatatgggccgtccccagccctgagcggttcttcatgcccctgtacaagggctgcagcggagacttcaagaaatgggtgggtgcacccttcactggctccagcctggagctgggaccctggagcccagaggtgccctccaccctggaggtgtacagctgccacccaccacggagcccggccaagaggctgcagctcacggagctacaagaaccagcagagctggtggagtctgacggtgtgcccaagcccagcttctggccgacagcccagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccccggggacgaaggacccccccggagctacctccgccagtgggtggtcattcctccgccactttcgagccctggaccccaggccagctaa

SEQ ID NO: 11 - protein, 497 aa, Artificial Sequence
  Description of Artificial Sequence Synthetic polypeptide
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASDFFERHLEFEARTLSPGHTWEEAPLLTLKQKQEWICLETLTPDTQYEFQVRVKPLQGEFTTWSPWSQPLAFRTKPASDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 12 - DNA, 1494 bp, Artificial Sequence
  Description of Artificial Sequence Synthetic polynucleotide
atggcggcccctgctctgtcctggcgtctgcccctcctcatcctcctcctgcccctggctacctcttgggcatctgcagcggtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctccgacttctttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag

SEQ ID NO: 13 - protein, 132 aa, Artificial Sequence
  Description of Artificial Sequence Synthetic polypeptide
PTSSSTKKTQLQLSQLLVLLKAILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 14 - protein, 11 aa, Artificial Sequence
  Description of Artificial Sequence Synthetic peptide
DICLPRWGCLW

SEQ ID NO: 15 - protein, 133 aa, Artificial Sequence
  Description of Artificial Sequence Synthetic polypeptide
  MOD_RES (1)..(1): A or absent
  MOD_RES (2)..(2): P or absent
  MOD_RES (3)..(3): T, C, A, G, Q, E, N, D, R, K, P, or absent
  MOD_RES (4)..(4): S or absent
  MOD_RES (5)..(5): S or absent
  MOD_RES (6)..(6): S or absent
  MOD_RES (7)..(7): T or absent
  MOD_RES (8)..(8): K or absent
  MOD_RES (9)..(9): K or absent
  MOD_RES (13)..(13): Q, W or absent
  MOD_RES (14)..(14): L, M, W or absent
  MOD_RES (15)..(15): E, K, D, T, A, S, Q, H or absent
  MOD_RES (16)..(16): H, N or Q or absent
  MOD_RES (18)..(18): L, R, G, M, F, E, H, W, K, Q, S, V, I, Y, H, D or T
  MOD_RES (19)..(19): L, A, V, I or absent
  MOD_RES (20)..(20): D, T, S, M, L, or absent
  MOD_RES (22)..(22): Q, F, E, G, A, L, M, F, W, K, S, V, I, Y, H, R, N, D, T, F or absent
  MOD_RES (23)..(23): M, A, W, H, Y, F, Q, S, V, L, T, or absent
  MOD_RES (27)..(27): G, K, S or absent
  MOD_RES (38)..(38): R, W or G
  MOD_RES (39)..(39): M, L or V
  MOD_RES (42)..(42): F or K
  MOD_RES (51)..(51): T, I or absent
  MOD_RES (55)..(55): H or Y
  MOD_RES (74)..(74): Q, N, H, S
  MOD_RES (80)..(80): L, F or V
  MOD_RES (81)..(81): R, I, D, Y, T or absent
  MOD_RES (85)..(85): L or V
  MOD_RES (86)..(86): I or V
  MOD_RES (88)..(88): N, E or Q or absent
  MOD_RES (89)..(89): I or V
  MOD_RES (91)..(91): V, R or K
  MOD_RES (92)..(92): I or F
  MOD_RES (97)..(97): K or Q
  MOD_RES (104)..(104): M or A
  MOD_RES (109)..(109): D, C or a non-natural amino acid with an activated side chain
  MOD_RES (113)..(113): T or N
  MOD_RES (125)..(125): C, A or S
  MOD_RES (126)..(126): Q, H, M, K, C, D, E, G, I, R, S, or T
  MOD_RES (130)..(130): S, T or R
XXXXXXXXXTQLXXXXLXXXLXXILNXINNYKNPKLTXXLTXKFYMPKKAXELKXLQCLEEELKPLEEVLNLAXSKNFHXXPRDXXSXXNXXVLELXGSETTFXCEYAXETAXIVEFLNRWITFXXSIIXTLT

SEQ ID NO: 16 - protein, 4 aa, Unknown
  Description of Unknown “DHHC” motif sequence
DHHC

SEQ ID NO: 17 - protein, 8 aa, Artificial Sequence
  Description of Artificial Sequence Synthetic peptide
  MOD_RES (2)..(2): V or I
  MOD_RES (4)..(4): Any amino acid
DXEXNPGP

SEQ ID NO: 18 - DNA, 20 bp, Artificial Sequence
  Description of Artificial Sequence Synthetic oligonucleotide
gtgtctttcaaagtagtggg

SEQ ID NO: 19 - DNA, 20 bp, Artificial Sequence
  Description of Artificial Sequence Synthetic oligonucleotide
ccacagatgcaacataagct

SEQ ID NO: 20 - DNA, 20 bp, Artificial Sequence
  Description of Artificial Sequence Synthetic oligonucleotide
caggtgtctttcaaagtagt

SEQ ID NO: 21 - DNA, 151 bp, Artificial Sequence
  Description of Artificial Sequence Synthetic polynucleotide
gcccccatctccctccaagttgtccacgtggagacccacagatgcaacataagctgggaaatctcccaagcatccgacttctttgaaagacacctggagttcgaggcccggacgctgtccccaggccacacctgggaggtgagaatgtggc

SEQ ID NO: 22 - DNA, 138 bp, Artificial Sequence
  Description of Artificial Sequence Synthetic polynucleotide
gcccccatctccctccaagttgtccacgtggagacccacaggtgcaacatatcctgggaaatctcccaagcctccgacttctttgaaagacacctggagttcgaggcccggacgctgtccccaggccacacctgggag

SEQ ID NO: 23 - DNA, 121 bp, Artificial Sequence
  Description of Artificial Sequence Synthetic polynucleotide
gcccccatctccctccaagttgtccacgtggagacccacagatgcaacataagctgggaaatctcccaagccagcgacttctttgaaagacacctggagttcgaggcccggacgctgtccc

SEQ ID NO: 24 - DNA, 151 bp, Artificial Sequence
  Description of Artificial Sequence Synthetic polynucleotide
gccacattctcacctcccaggtgtggcctggggacagcgtccgggcctcgaactccaggtgtctttcaaagaagtcggatgcttgggagatttcccagcttatgttgcatctgtgggtctccacgtggacaacttggagggagatgggggc

SEQ ID NO: 25 - DNA, 119 bp, Artificial Sequence
  Description of Artificial Sequence Synthetic polynucleotide
gccacattctcacctcccaggtgtggcctggggacagcgtccgggcctcgaactccaggtgtctttcaaagaagtcggaggcttgggagatttcccaggatatgttgcacctgtgggtc

SEQ ID NO: 26 - DNA, 105 bp, Artificial Sequence
  Description of Artificial Sequence Synthetic polynucleotide
gccacattctcacctcccaggtgtggcctggggacagcgtccgggcctcgaactccaggtgtctttcaaagaagtcgctggcttgggagatttcccagcttatgt

SEQ ID NO: 27 - DNA, 151 bp, Artificial Sequence
  Description of Artificial Sequence Synthetic polynucleotide
gcccccatctccctccaagttgtccacgtggagacccacagatgcaacataagctgggaaatctcccaagcatccgacttctttgaaagacacctcgagttcgaggcccggacgctgtccccaggccacacctgggaggtgagaatgtggc

SEQ ID NO: 28 - DNA, 145 bp, Artificial Sequence
  Description of Artificial Sequence Synthetic polynucleotide
gcccccatctccctccaagttgtccacgtggagacccacaggtgcaacatatcctgggaaatctcccaagcctccgacttctttgaaagacacctcgagttcgaggcccggacgctgtccccaggccacacctgggaggtgagaa

SEQ ID NO: 29 - DNA, 126 bp, Artificial Sequence
  Description of Artificial Sequence Synthetic polynucleotide
gcccccatctccctccaagttgtccacgtggagacccacagatgcaacataagctgggaaatctcccaagccagcgacttctttgaaagacacctcgagttcgaggcccggacgctgtccccaggc

SEQ ID NO: 30 - DNA, 151 bp, Artificial Sequence
  Description of Artificial Sequence Synthetic polynucleotide
gccacattctcacctcccaggtgtggcctggggacagcgtccgggcctcgaactcgaggtgtctttcaaagaagtcggatgcttgggagatttcccagcttatgttgcatctgtgggtctccacgtggacaacttggagggagatgggggc

SEQ ID NO: 31 - DNA, 129 bp, Artificial Sequence
  Description of Artificial Sequence Synthetic polynucleotide
gccacattctcacctcccaggtgtggcctggggacagcgtccgggcctcgaactcgaggtgtctttcaaagaagtcggaggcttgggagatttcccaggatatgttgcacctgtgggtctccacgtgga

SEQ ID NO: 32 - DNA, 114 bp, Artificial Sequence
  Description of Artificial Sequence Synthetic polynucleotide
gccacattctcacctcccaggtgtggcctggggacagcgtccgggcctcgaactcgaggtgtctttcaaagaagtcgctggcttgggagatttcccagcttatgttgcatctgt

SEQ ID NO: 33 - DNA, 340 bp, Artificial Sequence
  Description of Artificial Sequence Synthetic polynucleotide
agcatgtgccaggagggaggagtggcaggtacaaagtgggaggtggaggtcatggctgactgctctcccctaccccctgttgtctccagttcgcctgatggcccccatctccctccaagttgtccacgtggagacccacagatgcaacataagctgggaaatctcccaagcctcccactactttgaaagacacctggagttcgaggcccggacgctgtccccaggccacacctgggaggtgagaatgtggcccagtccgtacctgccttggttcctccttcctgtcctggtccatgtcctgtccctttttcattgtgtcgaggtcccctcagccccaggt

SEQ ID NO: 34 - protein, 50 aa, Artificial Sequence
  Description of Artificial Sequence Synthetic polypeptide
LRLMAPISLQVVHVETHRCNISWEISQASHYFERHLEFEARTLSPGHTWE

(19) United States
(12) Patent Application Publication
(10) Pub. No.: US 2023/0374454 A1
(43) Pub. Date: Nov. 23, 2023

(54) HUMAN IMMUNE CELLS GENOMICALLY MODIFIED TO EXPRESS ORTHOGONAL RECEPTORS

(71) Applicant: SYNTHEKINE, INC., Menlo Park, CA (US)

(72) Inventors: Paul-Joseph PENAFLOR ASPURIA, Menlo Park, CA (US); Martin OFT, Menlo Park, CA (US)

(21) Appl. No.: 17/916,739

(22) PCT Filed: Apr. 6, 2021
(86) PCT No.: PCT/US2021/026050

§ 371 Date: Oct. 3, 2022

Related U.S. Application Data
(60) Provisional application No. 63/005,975, filed on Apr. 6, 2020.

(51) Int. Cl. C12N 5/0783; C07K 14/705; C12N 15/86

(52) U.S. Cl. CPC C12N 5/0636; C07K 14/70596; C12N 15/86; C12N 2510/00; C12N 2310/20

(57) ABSTRACT

Human lymphocytes or myeloid cells comprising a polynucleotide encoding an engineered orthogonal human CD122, wherein the lymphocyte or myeloid does express native human CD122 are provided.

### CROSS-REFERENCE TO RELATED PATENT APPLICATIONS

The present patent application is a 371 U.S. National Phase of PCT International Application No. PCT/US2021/026050, filed Apr. 6, 2021, which claims priority to U.S. Provisional Patent Application No. 63/005,975, filed Apr. 6, 2020, which is incorporated by reference for all purposes.

### SEQUENCE LISTING

The instant application contains a Sequence Listing which has been submitted electronically in ASCII format and is hereby incorporated by reference in its entirety. Said ASCII copy, created on May 19, 2021, is named 106249-1243524-003110PC SL.txt and is 51,843 bytes in size.

### BACKGROUND OF THE INVENTION

The controlled manipulation of the differentiation, development and proliferation of cells, particularly engineered immune cells, is of significant clinical interest. In particular, T cells have been engineered for use in therapeutic applications such as the recognition and killing of cancer cells, intracellular pathogens and cells involved in autoimmunity. The use of engineered cell therapies in the treatment of cancer is facilitated by the selective activation and expansion of engineered cells (such as T cells) to provide specific functions and are directed to selectively attack cancer cells. In some examples of adoptive immunotherapy, T cells are isolated from the blood or tumor tissue of a subject, processed ex vivo, and re-infused into the subject. Compositions and methods that enable selective activation and/or proliferation of engineered cell immune cells are therefore desirable.

A challenge with the manufacture of cell therapy products for use in adoptive cell transfer (ACT) protocols is that such ‘living drugs’ require close control of their environment to preserve viability and functionality. In practice, isolated cells, whether derived from a patient (autologous) or from a single donor source that is not the patient (allogeneic), begin to lose function rapidly following removal from a subject or controlled culture conditions. Successful maintenance of the viability of the isolated cells while outside the subject or controlled culture conditions enables the isolated cells to maintain or return to functionality for reinsertion into the cell product manufacturing workflow or into patients. Additionally, successful maintenance of the viability of the engineered cells following administration of the engineered cells (i.e., persistence of the viable engineered cells) in a subject facilitates the clinical response to such cell therapy.

A challenge with the clinical application of engineered cell therapies is to maintain the viability of such engineered cells to maximize their therapeutic effectiveness. For example, in the case of the clinical applications of engineered T cells (e.g., CAR-T cells) the common means to maintain the viability of the engineered cells following administration to the subject is the systemic administration of the pluripotent cytokine, interleukin-2 usually in the form of aldesleukin (Proleukin®), a human IL2 analog having desAla1 and C125S modifications. In typical clinical practice of adoptive cell therapy with TILs or CAR-T cells, shortly after infusion of the TILS or CAR-T cells, the patient receives intravenous high-dose IL2 (720,000 IU/kg) every 8 hours until maximal tolerance. This subsequent support with IL2 is thought to further enhance the survival and clinical efficacy of the cell product.

However, the systemic administration of IL2 is associated with non-specific stimulatory effects beyond the population of engineered cells and is associated, particularly in high doses, with significant toxicity in human subjects. The effect of high dose IL2 typically used in ACT supportive regimens is documented to result in significant toxicities. The most prevalent side effects observed from the use of IL2 supportive therapy following adoptive cell transfer (ACT) include chills, high fever, hypotension, oliguria, and edema due to the systemic inflammatory and capillary leak syndrome as well as reports of autoimmune phenomena such as vitiligo or uveitis. Furthermore, IL2 has a short lifespan in vivo which requires that the IL2 be dosed frequently to maintain the engineered T cells in an activated state.

Although cells resulting from the administration of an ACT regimen may be detectable for months or even years following the administration of the cell product, a significant fraction (in some instances, the majority) of the administered cells lapse into a quiescent or exhausted state and demonstrate reduced therapeutic efficacy. Such loss of activity of the adoptively transferred cells frequently correlates with a loss of clinical efficacy including relapse or recurrence of the neoplastic disease. Consequently, a challenge to cell-based therapies is to confer a desired regulatable behavior into the transferred cells that is protected from endogenous signaling pathways, that exhibits minimal cross reactivity with non-targeted endogenous cells, and that can be selectively controlled following administration of the engineered cell population to a subject.

Additionally, during the ex vivo preparation of cells for use in ACT treatment regimens, a mixed population of isolated immune cells are frequently stimulated with IL2. Due to the pleiotropic nature of IL2 in the activation of immune cells, the culture of a mixed population of immune cells in the presence of IL2 leads to the expansion of not just the desired therapeutically useful cells (e.g., CAR-T cells or antigen experienced TILs) in the cell population but also the expansion of a variety of other types of immune cells in the population from the isolated tissue (e.g., neoplasm or blood) sample which do not contribute to the clinical benefit of the cell product and potentially contribute to toxicity. Consequently, current ex vivo expansion methods for the preparation of cells useful in ACT treatment regimens frequently result in cell products in which the desired subpopulation of therapeutically useful cells is contaminated with undesired cells resulting in a suboptimal cell product. As toxicity remains a significant issue with ACT, there is a need in the art for methods that enable the preparation of a cell product comprising a more homogeneous population enriched for the desired efficacious cells for use in ACT therapy.

CD122 is a component of the intermediate and high affinity IL2 receptor complexes. Sockolosky, et al. (Science (2018) 359: 1037-1042) and Garcia, et al. (United States Patent Application Publication US2018/0228841A1 published Aug. 16, 2018) describe an orthogonal IL2/CD122 ligand/receptor system to facilitate selective stimulation of cells engineered to express the orthogonal CD122. The present patent application incorporates by reference the disclosures of WO 2019/104092 and US 2018-0228842 A1) in their entireties. The contact of engineered immune cells that express an orthogonal CD122 with a corresponding orthogonal cognate ligand for such orthogonal CD122 (“IL2 ortholog”) facilitates specific activation and/or proliferation of such engineered immune cells that express the orthogonal CD122. In particular the orthogonal IL2 receptor ligand complex provides for selective activation and/or expansion of cells engineered to express the orthogonal receptor in a mixed population of cells, in particular a mixed population of T cells.

### BRIEF SUMMARY OF THE INVENTION

The present disclosure provides methods and compositions useful in the practice of adoptive cell therapy.

In some embodiments, the present disclosure provides an engineered human immune cell comprising a genomically-integrated polynucleotide encoding an orthogonal human CD122 (hoCD122) polypeptide. In some embodiments, the present disclosure provides an engineered human immune cell comprising a genomically-integrated polynucleotide encoding an hoCD122 operably linked to at least one expression control sequence functional in the human immune cell to effect expression of hoCD122 in the engineered human immune cell. In some embodiments, the engineered human immune cell expressing hoCD122 also expresses the wild-type human CD122. In other embodiments, the engineered human immune genomically modified to express hoCD122 does not express wild-type human CD122.

In some embodiments, the present disclosure provides an engineered human immune cell comprising a genomically-integrated polynucleotide encoding an orthogonal chimeric receptor (“OCR”) operably linked to at least one expression control sequence functional in the human immune cell to effect expression of the orthogonal chimeric receptor in the engineered human immune cell, the orthogonal chimeric receptor comprising an extracellular domain of an orthogonal hCD122 operably linked to an intracellular domain (ICD) of a heterologous receptor subunit including but not limited to the ICD from the IL-4 receptor alpha subunit (IL-4Rα), the IL-7 receptor alpha subunit (IL-7Rα), the IL-9 receptor alpha subunit (IL-9Rα), the IL-15R receptor alpha subunit (IL-15Rα), IL-21 receptor (IL-21R) or the erythropoietin receptor (EpoR), or a functional fragment thereof. In some embodiments, the engineered human immune cell genomically modified to express the orthogonal chimeric receptor further expresses the wild-type hCD122. In other embodiments, the engineered human immune cell comprising a genomically integrated polynucleotide encoding an orthogonal chimeric receptor does not express wild-type human CD122.

In some embodiments, the polynucleotide encoding an engineered orthogonal hCD122 is inserted in place of the endogenous hCD122 locus in the genome of the immune cell. In some embodiments, the hoCD122 is modified at one or more residues selected from R41, R42, Q70, K71, T73, T74, V75, 5132, H133, Y134, F135, E136, and Q214 relative to wild-type hCD122. In some embodiments, the hoCD122 is modified at positions H133 and Y134 relative to wild-type hCD122. In some embodiments, the engineered hoCD122 comprises an amino acid sequence at least 95% identical to SEQ ID NO:1. In some embodiments, the hoCD122 comprises SEQ ID NO:1 comprising the H133D and Y134F substitutions.

In some embodiments, the present disclosure provides an engineered human immune cell comprising a genomically-integrated polynucleotide encoding an orthogonal chimeric receptor (OCR) operably linked to at least one expression control sequence functional in the engineered human immune cell to effect expression of the OCR in the engineered human immune cell, the OCR comprising an extracellular domain (ECD) of an hoCD122 (or functional fragment thereof) operably linked to an intracellular domain (ICD) of a heterologous receptor subunit including but not limited to the ICD of the IL-4 receptor alpha subunit (IL-4Rα), the IL-7 receptor alpha subunit (IL-7Rα), the IL-9 receptor alpha subunit (IL-9Rα), the IL-15R receptor alpha subunit (IL-15Rα), IL-21 receptor (IL-21R) or the erythropoietin receptor (EpoR), or a functional fragment thereof. In some embodiments, the ECD of the chimeric receptor comprises the ECD of hoCD122 modified at one or more residues selected from R41, R42, Q70, K71, T73, T74, V75, 5132, H133, Y134, F135, E136, and Q214 relative to wild-type human CD122 ECD. In some embodiments, the engineered human immune cell genomically modified to express the OCR further expresses the wild-type hCD122. In other embodiments, the engineered human immune cell comprising a genomically integrated polynucleotide encoding the OCR does not express wild-type hCD122

In some embodiments, the human immune cell expresses a chimeric antigen receptor (CAR). In some embodiments, the CAR is selected from the group consisting of a CD19 chimeric antigen receptor (CAR), a B-Cell Maturation Antigen (BCMA) CAR, a CD123 CAR, a CD20 CAR, a CD22 CAR, a CD30 CAR, a CD70 CAR, a Lewis Y CAR, a GD3 CAR, a GD3 CAR, a mesothelin CAR, a ROR CAR, a CD44 CAR, a CD171 CAR, a EGP2 CAR, a EphA2 CAR, a ErbB2 CAR, a ErbB3/4 CAR, a FAP CAR, a FAR CAR, a IL11Ra CAR, a PSCA CAR, a PSMA CAR and a NCAM CAR.

In some embodiments, the engineered human immune cell expressing the hoCD122 or OCR is deleted for one or more of T cell receptor alpha (TCRA), T cell receptor beta (TCRB), PD-1, cytotoxic T-lymphocyte-associated protein 4 (CTLA4) or beta2 microglobulin (B2M).

In some embodiments, the engineered human immune cell expressing the hoCD122 or OCR is a T-cell. In some embodiments, the T-cell is a NK cell. In some embodiments, the T-cell is a tumor infiltrating lymphocyte (TIL). In some embodiments, the human immune cell is a Treg cell.

In some embodiments, the disclosure provides a method activating and/or enhancing proliferation the human immune cell genomically modified to express the hoCD122 or OCR as described above or elsewhere herein. In some embodiments, the method comprises contacting the engineered human immune cell engineered to express the hoCD122 or OCR with an orthogonal human IL-2 (hoIL2), wherein the contacting results of activation and/or proliferation of the engineered human immune cell.

In some embodiments, the disclosure provides a method of making a human immune cell comprising a polynucleotide encoding an engineered hoCD122 or OCR in place of the endogenous human CD122 locus. In some embodiments, the method comprises isolating a population of human immune cells and contacting the population of human immune cells with a polynucleotide in the endogenous human CD122 locus of the human immune cell, thereby making a human immune cell comprising a polynucleotide encoding an engineered hoCD122 in place of the endogenous human CD122 locus. In some embodiments, the introducing further comprises introducing a chimeric antigen receptor (CAR)-encoding polynucleotide into the human immune cell. In some embodiments, the polynucleotide encoding an engineered hoCD122 and the CAR-encoding polynucleotide are each part of a nucleic acid introduced into the human immune cell. In some embodiments, the engineered hoCD122 and the CAR are encoded as a single fusion protein separated by a self-cleaving peptide sequence. In some embodiments, the polynucleotide encoding an engineered hoCD122 and the CAR-encoding polynucleotide are separate nucleic acids introduced into the lymphocyte within a day of each other.

In some embodiments, the hoCD122 is modified at one or more residues selected from R41, R42, Q70, K71, T73, T74, V75, 5132, H133, Y134, F135, E136, and Q214 relative to native human CD122. In some embodiments, the hoCD122 is modified at H133 and Y134 relative to native human CD122 In some embodiments, the engineered hoCD122 comprises an amino acid sequence at least 95% identical to SEQ ID NO:1. In some embodiments, the engineered hoCD122 comprises SEQ ID NO:1 modified to have H133D and Y134F substitutions.

In some embodiments, the introducing comprises causing introduction of the polynucleotide by homology directed repair (HDR). In some embodiments, the introducing comprises introducing a clustered regularly interspaced short palindromic repeats (CRISPR) system into the human immune cell that cleaves in the endogenous human CD122 locus. In some embodiments, the system is a CRISPR/Cas9 or CRISPR/Cas12a system. In some embodiments, the introducing comprises introducing a transcription activator-like effector nuclease (TALEN) or zinc finger nuclease into the lymphocyte or myeloid cell that cleaves in the endogenous human CD122 locus. In some embodiments, the introducing comprises introducing a viral vector comprising the polynucleotide into the human immune cell.

In some embodiments, the method further comprises selectively expanding human immune cells (e.g., including but not limited to a lymphocyte or myeloid cells) comprising the engineered hoCD122 by contacting the immune cells with an orthogonal human IL-2. In some embodiments, the human immune cell expresses a chimeric antigen receptor (CAR) and further comprising selectively expanding the immune cells comprising the engineered hoCD122 by contacting the immune cells with a ligand than specifically binds to the extracellular domain (ECD) of the CAR. In some embodiments, the expanding further comprises contacting the human immune cells (e.g., including but not limited to a lymphocyte or myeloid cells) with an anti-CD28 antibody, an anti-CD3 antibody, or both.

In some embodiments, the providing comprises obtaining the human immune cells (e.g., including but not limited to a lymphocyte or myeloid cells) from a human.

In some embodiments, the human immune cells (e.g., including but not limited to a lymphocyte or myeloid cells) are introduced into a human following the introducing of the polynucleotide in the endogenous human CD122 locus of the immune cell. In some embodiments, the immune cells are autologous to the human. In some embodiments, the immune cells are allogeneic to the human.

In some embodiments, the disclosure provides a nucleic acid comprising a homology directed repair (HDR) template comprising a polynucleotide encoding an engineered hoCD122 comprising homology arms for insertion into an endogenous human locus. In some embodiments, the polynucleotide encoding an engineered hoCD122 comprises one or more mutation relative to the endogenous human CD122 locus such that one or more CRISPR protospacer adjacent motif (PAM) site is eliminated, optionally wherein the mutation results in a silent codon change. In some embodiments, the HDR template further comprises a CAR-encoding polynucleotide. In some embodiments, the engineered hoCD122 and the CAR are encoded as a single fusion protein separated by a self-cleaving peptide sequence.

In some embodiments, the disclosure provides a composition comprising the nucleic acid of any one of claims 33-36 and (i) a nuclease targeted to an endogenous human CD122 locus, (ii) a polynucleotide encoding the nuclease targeted to an endogenous human CD locus or (iii) a viral vector targeted to an endogenous human CD122 locus. In some embodiments, the nuclease is a clustered regularly interspaced short palindromic repeats (CRISPR) nuclease. In some embodiments, the nuclease is a CRISPR/Cas9 or CRISPR/Cas12a nuclease. In some embodiments, the nuclease is a transcription activator-like effector nuclease (TALEN) or zinc finger nuclease.

### BRIEF DESCRIPTION OF THE DRAWINGS

FIG. 1 depicts the genomic region surrounding an orthogonal mutation region of the CD122 coding sequence. H133 and D134 are above the short arrow in the third section below. Numbers are relative to the IL2RB gene locus. FIG. 1 discloses SEQ ID NOS 33-34, respectively, in order of appearance.

### DEFINITIONS

In order for the present disclosure to be more readily understood, certain terms and phrases are defined below as well as throughout the specification. The definitions provided herein are non-limiting and should be read in view of the knowledge of one of skill in the art would know.

Before the present methods and compositions are described, it is to be understood that this invention is not limited to particular method or composition described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; l or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; PCR=polymerase chain reaction; NHS=N-hydroxysuccinimide; HSA=human serum albumin; MSA=mouse serum albumin; DMEM=Dulbeco's Modification of Eagle's Medium; GC=genome copy; EDTA=ethylenediaminetetraacetic acid.

It will be appreciated that throughout this disclosure reference is made to amino acids according to the single letter or three letter codes. For the reader's convenience, the single and three letter amino acid codes are provided in Table 1 below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Unless otherwise indicated, the following terms are intended to have the meaning set forth below. Other terms are defined elsewhere throughout the specification.

Activate: As used herein the term “activate” is used in reference to a receptor or receptor complex to reflect the biological effect of the binding of an agonist ligand to the receptor. For example, it is said that the binding of an IL2 agonist to the IL2 receptor “activates” the receptor to result in one or more intracellular biological effects (e.g., phosphorylation of STAT5).

Activity: As used herein, the term “activity” is used with respect to a molecule to describe a property of the molecule with respect to a test system or biological function such as the degree of binding of the molecule to another molecule. Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a biological activity per unit of administered agent such as [catalytic activity]/[mg protein], [immunological activity]/[mg protein], international units (IU) of activity, [STATS or STAT3 phosphorylation]/[mg protein], [T-cell proliferation]/[mg protein], plaque forming units (pfu), etc. The term “proliferative activity” encompasses an activity that promotes cell division including dysregulated cell division as that observed in neoplastic diseases, inflammatory diseases, fibrosis, dysplasia, cell transformation, metastasis, and angiogenesis.

Administer: The terms “administration” and “administer” are used interchangeably herein to refer the act of contacting a subject, including contacting a cell, tissue, organ, or biological fluid in vitro, in vivo or ex vivo of the subject, with an agent (e.g., an orthologonal IL2 ligand, a cell expressing an orthogonal receptor, a CAR-T cell including a CAR-T cell expressing and orthogonal receptor, a chemotherapeutic agent, an antibody, or modulator or a pharmaceutical formulation comprising one or more of the foregoing). Administration of an agent may be achieved through any of a variety of art recognized methods including but not limited to the topical, intravascular injection (including intravenous or intraarterial infusion), intradermal injection, subcutaneous injection, intramuscular injection, intraperitoneal injection, intracranial injection, intratumoral injection, transdermal, transmucosal, iontophoretic delivery, intralymphatic injection, intragastric infusion, intraprostatic injection, intravesical infusion (e.g., bladder), respiratory inhalers, intraocular injection, intraabdominal injection, intralesional injection, intraovarian injection, intracerebral infusion or injection, intracerebroventricular injection (ICVI), and the like. The term “administration” includes contact of an agent to the cell, tissue or organ as well as the contact of an agent to a fluid, where the fluid is in contact with the cell.

Affinity: As used herein the term “affinity” refers to the degree of specific binding of a first molecule (e.g. a ligand) to a second molecule (e.g. a receptor) and is measured by the binding kinetics expressed as K_{d}, a ratio of the dissociation constant between the molecule and the its target (K_{off}) and the association constant between the molecule and its target (Kₒₙ).

Antibody: As used herein, the term “antibody” refers collectively to: (a) glycosylated and non-glycosylated the immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, camelids, antibodies, human antibodies. The term antibody includes so called “heavy chain antibodies” or “VHHs” or “Nanobodies®” as typically obtained from immunization of camelids (including camels, llamas and alpacas (see, e.g. Hamers-Casterman, et al. (1993) Nature 363:446-448). Antibodies having a given specificity may also be derived from non-mammalian sources such as VHHs obtained from immunization of cartilaginous fishes including, but not limited to, sharks. The term “antibody” encompasses antibodies isolatable from natural sources or from animals following immunization with an antigen and as well as engineered antibodies including monoclonal antibodies, bispecific antibodies, tri-specific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term antibody includes both the parent antibody and its derivatives such as affinity matured, veneered, CDR grafted (including CDR grafted VHHs), humanized, camelized (in the case of non-camel derived VHHs), or binding molecules comprising binding domains of antibodies (e.g., CDRs) in non-immunoglobulin scaffolds. The term “antibody” is not limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries or chemically synthesized (e.g., solid phase protein synthesis). In one embodiment, an “antibody” is a mammalian immunoglobulin. In some embodiments, the antibody is a “full length antibody” comprising variable and constant domains providing binding and effector functions. In most instances, a full-length antibody comprises two light chains and two heavy chains, each light chain comprising a variable region and a constant region. In some embodiments the term “full length antibody” is used to refer to conventional IgG immunoglobulin structures comprising two light chains and two heavy chains, each light chain comprising a variable region and a constant region providing binding and effector functions. The term antibody includes antibody conjugates comprising modifications to prolong duration of action such as fusion proteins or conjugation to polymers (e.g. PEGylated) as described in more detail below.

Biological Sample: As used herein, the term “biological sample” or “sample” refers to a sample obtained or derived from a subject. By way of example, a biological sample comprises a material selected from the group consisting of body fluids, blood, whole blood, plasma, serum, mucus secretions, saliva, cerebrospinal fluid (CSF), bronchoalveolar lavage fluid (BALF), fluids of the eye (e.g., vitreous fluid, aqueous humor), lymph fluid, lymph node tissue, spleen tissue, bone marrow, and an immunoglobulin enriched fraction derived from one or more of these tissues. In some embodiments, the sample is obtained from a subject who has been exposed to a therapeutic treatment regimen including a pharmaceutical formulation of an orthologonal IL2 ligand, such as repeatedly exposed to the same drug. In other embodiments, the sample is obtained from a subject who has not recently been exposed to the orthologonal IL2 ligand or obtained from the subject prior to the planned administration of the orthologonal IL2 ligand.

Chimeric Antigen Receptor: As used herein, the terms “chimeric antigen receptor” and “CAR” are used interchangeably to refer to a chimeric polypeptide comprising multiple functional domains arranged from amino to carboxy terminus in the sequence: (a) an extracellular domain (ECD) comprising an antigen binding domain (ABD)), and optionally comprising a “hinge” domain, (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs useful in the practice of the present methods can be prepared in accordance with principles well known in the art. See e.g., Eshhaar, et al. U.S. Pat. No. 7,741,465 B1 issued Jun. 22, 2010; Sadelain, et al (2013) Cancer Discovery 3(4):388-398; Jensen and Riddell (2015) Current Opinions in Immunology 33:9-15; Gross, et al. (1989) PNAS(USA) 86(24):10024-10028; Curran, et al. (2012) J Gene Med 14(6):405-15. Examples of commercially available CAR-T cell products that may be modified to incorporate an orthogonal receptor of the present invention include axicabtagene ciloleucel (marketed as Yescarta® commercially available from Gilead Pharmaceuticals) and tisagenlecleucel (marketed as Kymriah® commercially available from Novartis).

Chimeric Antigen Receptor T Cell: As used herein, the terms “chimeric antigen receptor T-cell” and “CAR-T cell” are used interchangeably to refer to a T-cell that has been recombinantly modified to express a chimeric antigen receptor. In some embodiments as exemplified herein, a CAR-T cell may be engineered to express an orthogonal CD122 polypeptide. In some embodiments, the CAR-T cell is engineered to express an orthogonal human CD122 polypeptide (“hoCAR-T” cell).

Derived From: As used herein in the term “derived from”, in the context of an amino acid sequence or polynucleotide sequence (e.g., an amino acid sequence “derived from” an IL2 polypeptide), is meant to indicate that the polypeptide or nucleic acid has a sequence that is based on that of a reference polypeptide or nucleic acid (e.g., a naturally occurring IL2 polypeptide or an IL2-encoding nucleic acid), and is not meant to be limiting as to the source or method in which the protein or nucleic acid is made. By way of example, the term “derived from” includes homologs or variants of reference amino acid or DNA sequences.

Extracellular Domain: As used herein the term “extracellular domain” or its abbreviation “ECD” refers to the portion of a cell surface protein (e.g., a cell surface receptor) which is outside of the plasma membrane of a cell. The ECD may include the entire extra-cytoplasmic portion of a transmembrane protein, a cell surface or membrane associated protein, a secreted protein, a cell surface targeting protein, or a functional polypeptide fragment thereof comprising the ligand binding domain of the ECD.

Human CD122: As used herein, the terms “human CD122”, “hCD122,” “human interleukin-2 receptor beta”, “hIL2Rb”, “hIL2Rβ”, “hIL15Rβ” and “p′70-′75” are used interchangeably to refer to the hCD122 transmembrane protein wild-type consensus sequence (UniProtKB database as entry P14784, SEQ ID NO:1) and naturally occurring variants thereof. A nucleic acid sequence encoding the hCD122 consensus protein sequences is identified as Genbank accession numbers NM_000878. The hCD122 wild-type protein is expressed as a 551 amino acid protein, the first 26 amino acids comprising a signal sequence which is post-translationally cleaved in the mature 525 amino acid wild-type protein. Amino acids 27-240 (amino acids 1-214 of the mature wild-type protein) correspond to the extracellular domain, amino acids 241-265 (amino acids 225-239 of the mature wild-type protein) correspond to the transmembrane domain and amino acids 266-551 (amino acids 240-525 of the mature wild-type protein) correspond to the intracellular domain. As used herein, hCD122 wild-type protein includes naturally occurring variants of the hCD122 protein including the S57F and D365E amino acid substitutions. The amino acid sequence of one naturally occurring human CD122 variant is:

(SEQ ID NO: 1)
AVNGTSQFTC FYNSRANISC VWSQDGALQD TSCQVHAWPD
RRRWNQTCEL LPVSQASWAC NLILGAPDSQ KLTTVDIVTL
RVLCREGVRW RVMAIQDFKP FENLRLMAPI SLQVVHVETH
RCNISWEISQ ASHYFERHLE FEARTLSPGH TWEEAPLLTL
KQKQEWICLE TLTPDTQYEF QVRVKPLQGE FTTWSPWSQP
LAFRTKPAAL GKDTIPWLGH LLVGLSGAFG FIILVYLLIN
CRNTGPWLKK VLKCNTPDPS KFFSQLSSEH GGDVQKWLSS
PFPSSSFSPG GLAPEISPLE VLERDKVTQL LLQQDKVPEP
ASLSSNHSLT SCFTNQGYFF FHLPDALEIE ACQVYFTYDP
YSEEDPDEGV AGAPTGSSPQ PLQPLSGEDD AYCTFPSRDD
LLLFSPSLLG GPSPPSTAPG GSGAGEERMP PSLQERVPRD
WDPQPLGPPT PGVPDLVDFQ PPPELVLREA GEEVPDAGPR
EGVSFPWSRP PGQGEFRALN ARLPLNTDAY LSLQELQGQD
PTHL

When reference is made to modifications of hCD122 present in hCD122 variants, the numbering of residues of such hCD122 variants is made with reference to SEQ ID NO: 1.

Interleukin-2 or IL2: As used herein, the terms “interleukin-2” and “IL2” are used interchangeably refers to a naturally occurring IL2 polypeptide that possesses IL2 activity. In some embodiments, IL2 refers to mature wild-type human IL2 lacking it naturally occurring 20 amino acid signal peptide sequence. Mature wild-type human IL2 (hIL2) occurs as a 133 amino acid polypeptide as described in Fujita, et. al., PNAS USA, 80, 7437-7441 (1983). An amino acid sequence of naturally occurring variant of mature wild-type human IL2 (hIL2) is:

(SEQ ID NO: 2)
APTSSSTKKT QLQLEHLLLD LQMILNGINN YKNPKLTRML
TFKFYMPKKA TELKHLQCLE EELKPLEEVL NLAQSKNEHL
RPRDLISNIN VIVLELKGSE TTEMCEYADE TATIVEFLNR
WITFCQSIIS TLT

As used herein, when referencing amino acid residues modified or deleted in hIL2 variants, the numbering of such modified or deleted residues is based on the wild-type mature hIL2 sequence UniProt ID P60568 excluding the signal peptide which is the same as that of SEQ ID NO:2.

IL2 Activity: The term “IL2 activity” refers to one or more the biological effects on a cell in response to contacting the cell with an effective amount of an IL2 polypeptide. IL2 activity may be measured, for example, in a cell proliferation assay using CTLL-2 mouse cytotoxic T cells, see Gearing, A. J. H. and C. B. Bird (1987) in Lymphokines and Interferons, A Practical Approach. Clemens, M. J. et al. (eds): IRL Press. 295. The specific activity of Recombinant Human IL2 is approximately 2.1×10⁴ IU/μg, which is calibrated against recombinant human IL2 WHO International Standard (NIB SC code: 86/500). In some embodiments, for example when the IL2 orthogonal polypeptide of interest exhibits (or is engineered to possess) diminished affinity for CD25, IL2 activity may be assessed in human cells such as YT cells which are capable of signaling through the intermediate affinity CD122/CD132 receptor. An orthogonal human IL2 of the present disclosure may have less than 20%, alternatively less than about 10%, alternatively less than about 8%, alternatively less than about 6%, alternatively less than about 4%, alternatively less than about 2%, alternatively less than about 1%, alternatively less than about 0.5% of the activity of WHO International Standard (NIB SC code: 86/500) wild-type mature human IL2 when evaluated at similar concentrations in a comparable assay.

Immune Cell: The term “immune cell” as used herein refers to eukaryotic living, cells hematopoietic origin, including primary cells and cell lines derived therefrom, that participate in the in the initiation and/or execution of innate and/or adaptive immune response including but not limited to B cells, T cells, Natural Killer (NK) cells, NK T cells, cytotoxic lymphocytes (CTLs), regulatory T cells (Tregs), dendritic cells, killer dendritic cells, and mast cells. In some embodiments immune cell that may be isolated from a mammalian subject is a T cell from the group consisting of inflammatory T-lymphocytes, cytotoxic T-lymphocytes, regulatory T-lymphocytes or helper T-lymphocytes including tumor infiltrating lymphocytes (TILs), CD4+ T-lymphocytes and CD8+ T-lymphocytes, cytotoxic T lymphocytes (CTLs), a regulatory T cell (Tregs), including subsets of CD8+T lymphocytes of various phenotypes including T effector memory phenotype (Tem), central memory phenotype (Thin), terminally differentiated Tem and Tem cells that express CD45RA (Temra), tissue resident memory (Trm) cells, and peripheral 10 memory (Tpm) cells, CD8+ effector subtypes are characterized in accordance with the following markers as shown in Table 2 below:

TABLE 2
Markers of CD8+ Memory Phenotypes

| Subset | Phenotype |
|---|---|
| Tem | CCR7^{lo}/CD62L^{lo} |
| | Cx3Cr1^{hi}/CD27^{lo} |
| | CD127^{hi} |
| | CD27⁻/CD45RA⁻ (humans) |
| Tcm | CCR7^{hi}/CD62L^{hi} |
| | Cx3Cr1^{lo}/CD27^{hi} |
| | CD127^{hi} |
| | CD27⁺/CD45RA⁻ (humans) |
| Temra (humans) | CCR7⁻/CD27⁻/CD45RA⁺ |
| | CD127^{lo} |
| Trm | CD69^{hi}/CD103^{hi}/CD49a^{hi} |
| | (depending on tissue) |
| | CXCR3^{hi}/KLRG1^{lo}/CCR7^{lo}/ |
| | CD62L^{lo}, CD127^{hi} |
| | Cx3Cr1^{lo/int} |
| Tpm | CCR7⁺/⁻/CD62L⁺/⁻/CD127^{hi} |
| | Cx3Cr1^{int}/CD27^{hi} |
| Others | CD27^{lo}/CD43^{lo} |
| | KLRG1^{hi}, CD127 [symbol] |

[symbol]

indicates data missing or illegible when filed

Martin, M. and Badinovac, V., Defining Memory CD8 T Cell (2018) Frontiers in Immunology 9:2692. In some embodiments, an immune cell refers to an immune cell isolated from a mammalian (e.g., human) subject. The term “primary cell(s)” refers to cells taken directly for living tissue and established for growth in vitro that have undergone few population doublings and are often considered more representative of the tissue since they are not transformed.

In An Amount Sufficient Amount to Effect a Change: As used herein the phrase “in an amount sufficient to effect a change” refers to the amount of a test agent sufficient to provide a detectable difference between a level of an indicator measured before (e.g., a baseline level) and after the application of the test agent to a system such as biological function evaluated in a cell based assay in response to the administration of a quantity of the test agent. “An amount sufficient to effect a change” may be sufficient to be a therapeutically effective amount but “in an amount sufficient to effect a change” may be more or less than a therapeutically effective amount.

In Combination With: As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g. an hoCD122^{pos}/wt hCD122^{neg} cell) is considered to be administered in combination with a second agent (e.g. hoIL2) if the biological effect resulting from the administration of the first agent persists in the subject at the time of administration of the second agent such that the therapeutic effects of the first agent and second agent overlap. For example, the hoCD122^{pos}/wt hCD122^{neg} cell is typically once while the hoIL2 ligand is typically administered more frequently, e.g. daily, BID, or weekly. However, the administration of the first agent (e.g. hoCD122^{pos}/wt hCD122^{neg} cell) provides a therapeutic effect over an extended time and the administration of the second agent (e.g. the hoIL2 ligand) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the hoIL2 ligand and the supplementary agent(s) are administered or applied sequentially, e.g., where one agent is administered prior to one or more other agents. In other embodiments, the hpIL2 mutein and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: As used herein the term “in need of prevention” refers to a judgment made by a physician or other caregiver with respect to a subject that the subject requires or will potentially benefit from preventative care. This judgment is made based upon a variety of factors that are in the realm of a physician's or caregiver's expertise.

As used herein the term “inhibitor” refers to a molecule that decreases, blocks, prevents, delays activation of, inactivates, desensitizes, or down-regulates, e.g., a gene, protein, ligand, receptor, or cell. An inhibitor can also be defined as a molecule that reduces, blocks, or inactivates a constitutive activity of a cell or organism.

Intracellular Domain of CD122: As used herein the terms “intracellular domain of the CD122” or “CD122 ICD” refer to the portion of a transmembrane spanning orthogonal receptor that is inside of the plasma membrane of a cell expressing such transmembrane spanning orthogonal receptor. The ICD may comprise one or more “proliferation signaling domain(s)” or “PSD(s)” which refers to a protein domain which signals the cell to enter mitosis and begin cell growth. Examples include the Janus kinases, including but not limited to, JAK1, JAK2, JAK3, Tyk2, Ptk-2, homologous members of the Janus kinase family from other mammalian or eukaryotic species, the IL2 receptor β and/or γ chains and other subunits from the cytokine receptor superfamily of proteins that may interact with the Janus kinase family of proteins to transduce a signal, or portions, modifications or combinations thereof. Examples of signals include phosphorylation of one or more STAT molecules including but not limited to one or more of STAT1, STAT3, STAT5a, and/or STAT5b.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex A ligand may comprise one domain of a polyprotein or fusion protein (e.g., either domain of an antibody/ligand fusion protein). The complex of a ligand and receptor is termed a “ligand-receptor complex.”

Myeloid Cell: As used herein, a “myeloid cell” refers to a cell that is derived from a myeloid progenitor cell. Exemplary myeloid cells include but are not limited to granulocytes, monocytes, erythrocytes, and platelets, as well as myeloid progenitor cells that are committed to the myeloid lineage.

Modulate: As used herein, the terms “modulate”, “modulation” and the like refer to the ability of a test agent to cause a response, either positive or negative or directly or indirectly, in a system, including a biological system, or biochemical pathway. The term modulator includes both agonists (including partial agonists, full agonists and superagonists) and antagonists.

Mutein: As used herein, the term “mutein” is used to refer to modified versions of wild type polypeptides comprising modifications to the primary structure (i.e. amino acid sequence) of such polypeptide. The term mutein may refer to the polypeptide itself, a composition comprising the polypeptide, or a nucleic acid sequence that encodes it. In some embodiments, the mutein polypeptide comprises from about one to about ten amino acid modifications relative to the parent polypeptide, alternatively from about one to about five amino acid modifications compared to the parent, alternatively from about one to about three amino acid modifications compared to the parent, alternatively from one to two amino acid modifications compared to the parent, alternatively a single amino acid modification compared to the parent. A mutein may be at least about 99% identical to the parent polypeptide, alternatively at least about 98% identical, alternatively at least about 97% identical, alternatively at least about 95% identical, or alternatively at least about 90% identical.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Numbered in accordance with hIL2: The term “numbered in accordance with IL2” as used herein refers to the identification of a location of particular amino acid with reference to the position at which that amino acid normally occurs in the mature sequence of the mature wild type hIL2, for example R81 refers to the eighty-first amino acid, arginine, that occurs in SEQ ID NO: 2.

Numbered in accordance with hCD122: The term “numbered in accordance with hCD122” as used herein refers to the identification of a location of particular amino acid with reference to the position at which that amino acid normally occurs in the mature sequence of the mature wild type hCD122 (SEQ ID NO:1).

Operably Linked: The term “operably linked” is used herein to refer to the relationship between molecules, typically polypeptides or nucleic acids, which are arranged in a construct such that each of the functions of the component molecules is retained although the operable linkage may result in the modulation of the activity, either positively or negatively, of the individual components of the construct. For example, the operable linkage of a polyethylene glycol (PEG) molecule to a wild-type protein may result in a construct where the biological activity of the protein is diminished relative to the wild-type molecule, however the two are nevertheless considered operably linked. Alternatively, in the context of a multi-domain receptor comprised of functional domains derived from heterologous sources (e.g., a CAR or OCR), the functional domains of the fusion protein are operably linked when a function characteristic of a first domain of the fusion protein (e.g. ligand binding to the ECD) modulates a function characteristic of a second domain of the fusion protein (e.g., intracellular signaling of the ICD). When the term “operably linked” is applied to the relationship of multiple nucleic acid sequences encoding differing functions, the multiple nucleic acid sequences when combined into a single nucleic acid molecule that, for example, when introduced into a cell using recombinant technology, provides a nucleic acid which is capable of effecting the transcription and/or translation of a particular nucleic acid sequence in a cell. For example, the nucleic acid sequence encoding a signal sequence may be considered operably linked to DNA encoding a polypeptide if it results in the expression of a preprotein whereby the signal sequence facilitates the secretion of the polypeptide; a promoter or enhancer is considered operably linked to a coding sequence if it affects the transcription of the sequence; or a ribosome binding site is considered operably linked to a coding sequence if it is positioned so as to facilitate translation. Generally in the context of nucleic acid molecules, the term “operably linked” means that the nucleic acid sequences being linked are contiguous, and, in the case of a secretory leader or associated subdomains of a molecule, contiguous and in reading phase. However, certain genetic elements such as enhancers may function at a distance and need not be contiguous with respect to the sequence to which they provide their effect but nevertheless may be considered operably linked.

Orthogonal Chimeric Receptor: As used herein, the terms “orthogonal chimeric receptor” or “OCR” are used interchangeably to refer a polypeptide the extracellular domain (ECD) of which is derived from an hoCD122 or functional subfragment thereof, operably linked to an intracellular domain (ICD) of a heterologous receptor subunit including but not limited to the ICD of from the IL-4 receptor alpha subunit (IL-4Rα), the IL-7 receptor alpha subunit (IL-7Rα), the IL-9 receptor alpha subunit (IL-9Rα), the IL-15R receptor alpha subunit (IL-15Rα), IL-21 receptor (IL-21R) or the erythropoietin receptor (EpoR), or a functional fragment thereof. The ECD and ICD of the OCR may be operably linked via a polypeptide sequence comprising the transmembrane domain of the receptor from which the ICD or ECD of the OCR are derived. In one embodiment, ICD or ECD of the OCR are operably linked via a polypeptide comprising the transmembrane domain of the receptor from which the ECD is derived. In one embodiment, ICD or ECD of the OCR are operably linked via a polypeptide comprising the transmembrane domain of the receptor from which the ICD is derived. Examples of OCRs are described in Garcia, et al., International Patent Application No. PCT/US2020/050232 published Mar. 18, 2021 as WO 2021/050752 and exemplified below.

OCR comprising a hoCD122 ECD and IL7ICD (hoCD122-IL7R) protein sequence:

(SEQ ID NO: 5)
MAAPALSWRLPLLILLLPLATSWASAAVNGTSQFTCFYNSRANI
SCVWSQDGALQDTSCQVHAWPDRRRWNQTCELLPVSQASWACNL
ILGAPDSQKLTTVDIVTLRVLCREGVRWRVMAIQDFKPFENLRL
MAPISLQVVHVETHRCNISWEISQASDFFERHLEFEARTLSPGH
TWEEAPLLTLKQKQEWICLETLTPDTQYEFQVRVKPLQGEFTTW
SPWSQPLAFRTKPANNSSGEMDPILLTISILSFFSVALLVILAC
VLWKKRIKPIVWPSLPDHKKTLEHLCKKPRKNLNVSFNPESFLD
CQIHRVDDIQARDEVEGFLQDTFPQQLEESEKQRLGGDVQSPNC
PSEDVVITPESFGRDSSLTCLAGNVSACDAPILSSSRSLDCRES
GKNGPHVYQDLLLSLGTTNSTLPPPFSLQSGILTLNPVAQGQPI
LTSLGSNQEEAYVTMSSFYQNQ

wherein residues 1-234 are derived from hoCD122 and residues 235-462 are derived from the ICD of the human IL-7Ra receptor (underlined) and can be encoded by the nucleic acid sequence

(SEQ ID NO: 6)
ATGGCGGCCCCTGCTCTGTCCTGGCGTCTGCCCCTCCTCATCCT
CCTCCTGCCCCTGGCTACCTCTTGGGCATCTGCAGCGGTGAATG
GCACTTCCCAGTTCACATGCTTCTACAACTCGAGAGCCAACATC
TCCTGTGTCTGGAGCCAAGATGGGGCTCTGCAGGACACTTCCTG
CCAAGTCCATGCCTGGCCGGACAGACGGCGGTGGAACCAAACCT
GTGAGCTGCTCCCCGTGAGTCAAGCATCCTGGGCCTGCAACCTG
ATCCTCGGAGCCCCAGATTCTCAGAAACTGACCACAGTTGACAT
CGTCACCCTGAGGGTGCTGTGCCGTGAGGGGGTGCGATGGAGGG
TGATGGCCATCCAGGACTTCAAGCCCTTTGAGAACCTTCGCCTG
ATGGCCCCCATCTCCCTCCAAGTTGTCCACGTGGAGACCCACAG
ATGCAACATAAGCTGGGAAATCTCCCAAGCCTCCgACTtCTTTG
AAAGACACCTGGAGTTCGAGGCCCGGACGCTGTCCCCAGGCCAC
ACCTGGGAGGAGGCCCCCCTGCTGACTCTCAAGCAGAAGCAGGA
ATGGATCTGCCTGGAGACGCTCACCCCAGACACCCAGTATGAGT
TTCAGGTGCGGGTCAAGCCTCTGCAAGGCGAGTTCACGACCTGG
AGCCCCTGGAGCCAGCCCCTGGCCTTCAGGACAAAGCCTGCAAA
TAATAGCTCAGGGGAGATGGATCCTATCTTACTAACCATCAGCA
TTTTGAGTTTTTTCTCTGTCGCTCTGTTGGTCATCTTGGCCTGT
GTGTTATGGAAAAAAAGGATTAAGCCTATCGTATGGCCCAGTCT
CCCCGATCATAAGAAGACTCTGGAACATCTTTGTAAGAAACCAA
GAAAAAATTTAAATGTGAGTTTCAATCCTGAAAGTTTCCTGGAC
TGCCAGATTCATAGGGTGGATGACATTCAAGCTAGAGATGAAGT
GGAAGGTTTTCTGCAAGATACGTTTCCTCAGCAACTAGAAGAAT
CTGAGAAGCAGAGGCTTGGAGGGGATGTGCAGAGCCCCAACTGC
CCATCTGAGGATGTAGTCATCACTCCAGAAAGCTTTGGAAGAGA
TTCATCCCTCACATGCCTGGCTGGGAATGTCAGTGCATGTGACG
CCCCTATTCTCTCCTCTTCCAGGTCCCTAGACTGCAGGGAGAGT
GGCAAGAATGGGCCTCATGTGTACCAGGACCTCCTGCTTAGCCT
TGGGACTACAAACAGCACGCTGCCCCCTCCATTTTCTCTCCAAT
CTGGAATCCTGACATTGAACCCAGTTGCTCAGGGTCAGCCCATT
CTTACTTCCCTGGGATCAAATCAAGAAGAAGCATATGTCACCAT
GTCCAGCTTCTACCAAAACCAGTGA

OCR comprising a hoCD122 ECD and an IL9Ra ICD (hoCD122-IL9R) coding sequence:

(SEQ ID NO: 7)
MAAPALSWRLPLLILLLPLATSWASAAVNGTSQFTCFYNSRANI
SCVWSQDGALQDTSCQVHAWPDRRRWNQTCELLPVSQASWACNL
ILGAPDSQKLTTVDIVTLRVLCREGVRWRVMAIQDFKPFENLRL
MAPISLQVVHVETHRCNISWEISQASDFFERHLEFEARTLSPGH
TWEEAPLLTLKQKQEWICLETLTPDTQYEFQVRVKPLQGEFTTW
SPWSQPLAFRTKPAQRQGPLIPPWGWPGNTLVAVSIFLLLTGPT
YLLFKLSPRVKRIFYQNVPSPAMFFQPLYSVHNGNFQTWMGAHG
AGVLLSQDCAGTPQGALEPCVQEATALLTCGPARPWKSVALEEE
QEGPGTRLPGNLSSEDVLPAGCTEWRVQTLAYLPQEDWAPTSLT
RPAPPDSEGSRSSSSSSSSNNNNYCALGCYGGWHLSALPGNTQS
SGPIPALACGLSCDHQGLETQQGVAWVLAGHCQRPGLHEDLQGM
LLPSVLSKARSWTF

wherein residues 1-234 are derived from hoCD122 and residues 235-498 are derived from human IL-9R (underlined)

(SEQ ID NO: 8)
ATGGCGGCCCCTGCTCTGTCCTGGCGTCTGCCCCTCCTCATCCT
CCTCCTGCCCCTGGCTACCTCTTGGGCATCTGCAGCGGTGAATG
GCACTTCCCAGTTCACATGCTTCTACAACTCGAGAGCCAACATC
TCCTGTGTCTGGAGCCAAGATGGGGCTCTGCAGGACACTTCCTG
CCAAGTCCATGCCTGGCCGGACAGACGGCGGTGGAACCAAACCT
GTGAGCTGCTCCCCGTGAGTCAAGCATCCTGGGCCTGCAACCTG
ATCCTCGGAGCCCCAGATTCTCAGAAACTGACCACAGTTGACAT
CGTCACCCTGAGGGTGCTGTGCCGTGAGGGGGTGCGATGGAGGG
TGATGGCCATCCAGGACTTCAAGCCCTTTGAGAACCTTCGCCTG
ATGGCCCCCATCTCCCTCCAAGTTGTCCACGTGGAGACCCACAG
ATGCAACATAAGCTGGGAAATCTCCCAAGCCTCCgACTtCTTTG
AAAGACACCTGGAGTTCGAGGCCCGGACGCTGTCCCCAGGCCAC
ACCTGGGAGGAGGCCCCCCTGCTGACTCTCAAGCAGAAGCAGGA
ATGGATCTGCCTGGAGACGCTCACCCCAGACACCCAGTATGAGT
TTCAGGTGCGGGTCAAGCCTCTGCAAGGCGAGTTCACGACCTGG
AGCCCCTGGAGCCAGCCCCTGGCCTTCAGGACAAAGCCTGCACA
GAGACAAGGCCCTCTGATCCCACCCTGGGGGTGGCCAGGCAACA
CCCTTGTTGCTGTGTCCATCTTTCTCCTGCTGACTGGCCCGACC
TACCTCCTGTTCAAGCTGTCGCCCAGGGTGAAGAGAATCTTCTA
CCAGAACGTGCCCTCTCCAGCGATGTTCTTCCAGCCCCTCTACA
GTGTACACAATGGGAACTTCCAGACTTGGATGGGGGCCCACGGG
GCCGGTGTGCTGTTGAGCCAGGACTGTGCTGGCACCCCACAGGG
AGCCTTGGAGCCCTGCGTCCAGGAGGCCACTGCACTGCTCACTT
GTGGCCCAGCGCGTCCTTGGAAATCTGTGGCCCTGGAGGAGGAA
CAGGAGGGCCCTGGGACCAGGCTCCCGGGGAACCTGAGCTCAGA
GGATGTGCTGCCAGCAGGGTGTACGGAGTGGAGGGTACAGACGC
TTGCCTATCTGCCACAGGAGGACTGGGCCCCCACGTCCCTGACT
AGGCCGGCTCCCCCAGACTCAGAGGGCAGCAGGAGCAGCAGCAG
CAGCAGCAGCAGCAACAACAACAACTACTGTGCCTTGGGCTGCT
ATGGGGGATGGCACCTCTCAGCCCTCCCAGGAAACACACAGAGC
TCTGGGCCCATCCCAGCCCTGGCCTGTGGCCTTTCTTGTGACCA
TCAGGGCCTGGAGACCCAGCAAGGAGTTGCCTGGGTGCTGGCTG
GTCACTGCCAGAGGCCTGGGCTGCATGAGGACCTCCAGGGCATG
TTGCTCCCTTCTGTCCTCAGCAAGGCTCGGTCCTGGACATTCTA

OCR comprising a hoCD122 ECD and an IL21Ra ICD (hoCD122-IL21R) coding sequence:

(SEQ ID NO: 9)
MAAPALSWRLPLLILLLPLATSWASAAVNGTSQFTCFYNSRANI
SCVWSQDGALQDTSCQVHAWPDRRRWNQTCELLPVSQASWACNL
ILGAPDSQKLTTVDIVTLRVLCREGVRWRVMAIQDFKPFENLRL
MAPISLQVVHVETHRCNISWEISQASDFFERHLEFEARTLSPGH
TWEEAPLLTLKQKQEWICLETLTPDTQYEFQVRVKPLQGEFTTW
SPWSQPLAFRTKPAEELKEGWNPHLLLLLLLVIVFIPAFWSLKT
HPLWRLWKKIWAVPSPERFFMPLYKGCSGDFKKWVGAPFTGSSL
ELGPWSPEVPSTLEVYSCHPPRSPAKRLQLTELQEPAELVESDG
VPKPSFWPTAQNSGGSAYSEERDRPYGLVSIDTVTVLDAEGPCT
WPCSCEDDGYPALDLDAGLEPSPGLEDPLLDAGTTVLSCGCVSA
GSPGLGGPLGSLLDRLKPPLADGEDWAGGLPWGGRSPGGVSESE
AGSPLAGLDMDTFDSGFVGSDCSSPVECDFTSPGDEGPPRSYLR
QWVVIPPPLSSPGPQAS

Wherein residues 1-234 are derived from hoCD122 and residues 235-545 human IL-21R (underlined) and which is encoded by the polynucleotide of the sequence

(SEQ ID NO: 10)
ATGGCGGCCCCTGCTCTGTCCTGGCGTCTGCCCCTCCTCATCCT
CCTCCTGCCCCTGGCTACCTCTTGGGCATCTGCAGCGGTGAATG
GCACTTCCCAGTTCACATGCTTCTACAACTCGAGAGCCAACATC
TCCTGTGTCTGGAGCCAAGATGGGGCTCTGCAGGACACTTCCTG
CCAAGTCCATGCCTGGCCGGACAGACGGCGGTGGAACCAAACCT
GTGAGCTGCTCCCCGTGAGTCAAGCATCCTGGGCCTGCAACCTG
ATCCTCGGAGCCCCAGATTCTCAGAAACTGACCACAGTTGACAT
CGTCACCCTGAGGGTGCTGTGCCGTGAGGGGGTGCGATGGAGGG
TGATGGCCATCCAGGACTTCAAGCCCTTTGAGAACCTTCGCCTG
ATGGCCCCCATCTCCCTCCAAGTTGTCCACGTGGAGACCCACAG
ATGCAACATAAGCTGGGAAATCTCCCAAGCCTCCgACTtCTTTG
AAAGACACCTGGAGTTCGAGGCCCGGACGCTGTCCCCAGGCCAC
ACCTGGGAGGAGGCCCCCCTGCTGACTCTCAAGCAGAAGCAGGA
ATGGATCTGCCTGGAGACGCTCACCCCAGACACCCAGTATGAGT
TTCAGGTGCGGGTCAAGCCTCTGCAAGGCGAGTTCACGACCTGG
AGCCCCTGGAGCCAGCCCCTGGCCTTCAGGACAAAGCCTGCAGA
GGAGTTAAAGGAAGGCTGGAACCCTCACCTGCTGCTTCTCCTCC
TGCTTGTCATAGTCTTCATTCCTGCCTTCTGGAGCCTGAAGACC
CATCCATTGTGGAGGCTATGGAAGAAGATATGGGCCGTCCCCAG
CCCTGAGCGGTTCTTCATGCCCCTGTACAAGGGCTGCAGCGGAG
ACTTCAAGAAATGGGTGGGTGCACCCTTCACTGGCTCCAGCCTG
GAGCTGGGACCCTGGAGCCCAGAGGTGCCCTCCACCCTGGAGGT
GTACAGCTGCCACCCACCACGGAGCCCGGCCAAGAGGCTGCAGC
TCACGGAGCTACAAGAACCAGCAGAGCTGGTGGAGTCTGACGGT
GTGCCCAAGCCCAGCTTCTGGCCGACAGCCCAGAACTCGGGGGG
CTCAGCTTACAGTGAGGAGAGGGATCGGCCATACGGCCTGGTGT
CCATTGACACAGTGACTGTGCTAGATGCAGAGGGGCCATGCACC
TGGCCCTGCAGCTGTGAGGATGACGGCTACCCAGCCCTGGACCT
GGATGCTGGCCTGGAGCCCAGCCCAGGCCTAGAGGACCCACTCT
TGGATGCAGGGACCACAGTCCTGTCCTGTGGCTGTGTCTCAGCT
GGCAGCCCTGGGCTAGGAGGGCCCCTGGGAAGCCTCCTGGACAG
ACTAAAGCCACCCCTTGCAGATGGGGAGGACTGGGCTGGGGGAC
TGCCCTGGGGTGGCCGGTCACCTGGAGGGGTCTCAGAGAGTGAG
GCGGGCTCACCCCTGGCCGGCCTGGATATGGACACGTTTGACAG
TGGCTTTGTGGGCTCTGACTGCAGCAGCCCTGTGGAGTGTGACT
TCACCAGCCCCGGGGACGAAGGACCCCCCCGGAGCTACCTCCGC
CAGTGGGTGGTCATTCCTCCGCCACTTTCGAGCCCTGGACCCCA
GGCCAGCTAA

OCR comprising a hoCD122 ECD and an ICD derived from the Epo having the amino acid sequence:

(SEQ ID NO: 11)
MAAPALSWRLPLLILLLPLATSWASAAVNGTSQFTCFYNSRANI
SCVWSQDGALQDTSCQVHAWPDRRRWNQTCELLPVSQASWACNL
ILGAPDSQKLTTVDIVTLRVLCREGVRWRVMAIQDFKPFENLRL
MAPISLQVVHVETHRCNISWEISQASDFFERHLEFEARTLSPGH
TWEEAPLLTLKQKQEWICLETLTPDTQYEFQVRVKPLQGEFTTW
SPWSQPLAFRTKPASDLDPLILTLSLILVVILVLLTVLALLSHR
RALKQKIWPGIPSPESEFEGLFTTHKGNFQLWLYQNDGCLWWSP
CTPFTEDPPASLEVLSERCWGTMQAVEPGTDDEGPLLEPVGSEH
AQDTYLVLDKWLLPRNPPSEDLPGPGGSVDIVAMDEGSEASSCS
SALASKPSPEGASAASFEYTILDPSSQLLRPWTLCPELPPTPPH
LKYLYLVVSDSGISTDYSSGDSQGAQGGLSDGPYSNPYENSLIP
AAEPLPPSYVACS

wherein residues 1-234 are derived from hoCD122 and residues 235-497 are derived from human EpoR (underlined) and is encoded by the polynucleotide of the sequence:

(SEQ ID NO: 12)
ATGGCGGCCCCTGCTCTGTCCTGGCGTCTGCCCCTCCTCATCCT
CCTCCTGCCCCTGGCTACCTCTTGGGCATCTGCAGCGGTGAATG
GCACTTCCCAGTTCACATGCTTCTACAACTCGAGAGCCAACATC
TCCTGTGTCTGGAGCCAAGATGGGGCTCTGCAGGACACTTCCTG
CCAAGTCCATGCCTGGCCGGACAGACGGCGGTGGAACCAAACCT
GTGAGCTGCTCCCCGTGAGTCAAGCATCCTGGGCCTGCAACCTG
ATCCTCGGAGCCCCAGATTCTCAGAAACTGACCACAGTTGACAT
CGTCACCCTGAGGGTGCTGTGCCGTGAGGGGGTGCGATGGAGGG
TGATGGCCATCCAGGACTTCAAGCCCTTTGAGAACCTTCGCCTG
ATGGCCCCCATCTCCCTCCAAGTTGTCCACGTGGAGACCCACAG
ATGCAACATAAGCTGGGAAATCTCCCAAGCCTCCgACTtCTTTG
AAAGACACCTGGAGTTCGAGGCCCGGACGCTGTCCCCAGGCCAC
ACCTGGGAGGAGGCCCCCCTGCTGACTCTCAAGCAGAAGCAGGA
ATGGATCTGCCTGGAGACGCTCACCCCAGACACCCAGTATGAGT
TTCAGGTGCGGGTCAAGCCTCTGCAAGGCGAGTTCACGACCTGG
AGCCCCTGGAGCCAGCCCCTGGCCTTCAGGACAAAGCCTGCAAG
CGACCTGGACCCCCTCATCCTGACGCTCTCCCTCATCCTCGTGG
TCATCCTGGTGCTGCTGACCGTGCTCGCGCTGCTCTCCCACCGC
CGGGCTCTGAAGCAGAAGATCTGGCCTGGCATCCCGAGCCCAGA
GAGCGAGTTTGAAGGCCTCTTCACCACCCACAAGGGTAACTTCC
AGCTGTGGCTGTACCAGAATGATGGCTGCCTGTGGTGGAGCCCC
TGCACCCCCTTCACGGAGGACCCACCTGCTTCCCTGGAAGTCCT
CTCAGAGCGCTGCTGGGGGACGATGCAGGCAGTGGAGCCGGGGA
CAGATGATGAGGGCCCCCTGCTGGAGCCAGTGGGCAGTGAGCAT
GCCCAGGATACCTATCTGGTGCTGGACAAATGGTTGCTGCCCCG
GAACCCGCCCAGTGAGGACCTCCCAGGGCCTGGTGGCAGTGTGG
ACATAGTGGCCATGGATGAAGGCTCAGAAGCATCCTCCTGCTCA
TCTGCTTTGGCCTCGAAGCCCAGCCCAGAGGGAGCCTCTGCTGC
CAGCTTTGAGTACACTATCCTGGACCCCAGCTCCCAGCTCTTGC
GTCCATGGACACTGTGCCCTGAGCTGCCCCCTACCCCACCCCAC
CTAAAGTACCTGTACCTTGTGGTATCTGACTCTGGCATCTCAAC
TGACTACAGCTCAGGGGACTCCCAGGGAGCCCAAGGGGGCTTAT
CCGATGGCCCCTACTCCAACCCTTATGAGAACAGCCTTATCCCA
GCCGCTGAGCCTCTGCCCCCCAGCTATGTGGCTTGCTCTTAG.

Orthogonal Human IL2: The term “orthogonal hIL2” or “hoIL2” refers to a variant of hIL2 (SEQ ID NO:2) that selectively and specifically binds to the ECD of an orthogonal hCD122 receptor or OCR and result in intracellular signaling. Examples of hoIL2 molecules are provided in Formula 1 below.

Orthogonal Human CD122: As used herein, the terms “human orthogonal CD122” or “orthogonal human CD122” or “hoCD122” are used interchangeably to refers to a variant of the wild-type CD122 polypeptide that specifically binds to an orthogonal human IL2 (hoIL2). In some embodiments, the hoCD122 comprises amino acid substitutions at positions histidine 133 (H133) and tyrosine 134 (Y134) in the ECD of the hCD122 polypeptide. In some embodiments orthogonal CD122 comprises the amino acid substitutions at position 133 from histidine to aspartic acid (H133D), glutamic acid (H133E) or lysine (H133K) and/or amino acid substitutions at position 134 to from tyrosine to phenylalanine (Y134F), glutamic acid (Y134E), or arginine (Y134R). In some embodiments, the orthogonal CD122 is a hCD122 molecule having amino acid substitutions H133D and Y134F. One embodiment of an hoCD122 is provided as is a polypeptide of the sequence

(SEQ ID NO: 3)
MAAPALSWRLPLLILLLPLATSWASAAVNGTSQFTCFYNSRANI
SCVWSQDGALQDTSCQVHAWPDRRRWNQTCELLPVSQASWACNL
ILGAPDSQKLTTVDIVTLRVLCREGVRWRVMAIQDFKPFENLRL
MAPISLQVVHVETHRCNISWEISQASDFFERHLEFEARTLSPGH
TWEEAPLLTLKQKQEWICLETLTPDTQYEFQVRVKPLQGEFTTW
SPWSQPLAFRTKPAALGKDTIPWLGHLLVGLSGAFGFIILVYLL
INCRNTGPWLKKVLKCNTPDPSKFFSQLSSEHGGDVQKWLSSPF
PSSSFSPGGLAPEISPLEVLERDKVTQLLLQQDKVPEPASLSSN
HSLTSCFTNQGYFFFHLPDALEIEACQVYFTYDPYSEEDPDEGV
AGAPTGSSPQPLQPLSGEDDAYCTFPSRDDLLLFSPSLLGGPSP
PSTAPGGSGAGEERMPPSLQERVPRDWDPQPLGPPTPGVPDLVD
FQPPPELVLREAGEEVPDAGPREGVSFPWSRPPGQGEFRALNAR
LPLNTDAYLSLQELQGQDPTHLV

And a representative nucleic acid sequence encoding human orthogonal CD122 (hoCD122) of SEQ ID NO:3 is provided below:

(SEQ ID NO: 4)
ATGGCGGCCCCTGCTCTGTCCTGGCGTCTGCCCCTCCTCATCCT
CCTCCTGCCCCTGGCTACCTCTTGGGCATCTGCAGCGGTGAATG
GCACTTCCCAGTTCACATGCTTCTACAACTCGAGAGCCAACATC
TCCTGTGTCTGGAGCCAAGATGGGGCTCTGCAGGACACTTCCTG
CCAAGTCCATGCCTGGCCGGACAGACGGCGGTGGAACCAAACCT
GTGAGCTGCTCCCCGTGAGTCAAGCATCCTGGGCCTGCAACCTG
ATCCTCGGAGCCCCAGATTCTCAGAAACTGACCACAGTTGACAT
CGTCACCCTGAGGGTGCTGTGCCGTGAGGGGGTGCGATGGAGGG
TGATGGCCATCCAGGACTTCAAGCCCTTTGAGAACCTTCGCCTG
ATGGCCCCCATCTCCCTCCAAGTTGTCCACGTGGAGACCCACAG
ATGCAACATAAGCTGGGAAATCTCCCAAGCCTCCgACTtCTTTG
AAAGACACCTGGAGTTCGAGGCCCGGACGCTGTCCCCAGGCCAC
ACCTGGGAGGAGGCCCCCCTGCTGACTCTCAAGCAGAAGCAGGA
ATGGATCTGCCTGGAGACGCTCACCCCAGACACCCAGTATGAGT
TTCAGGTGCGGGTCAAGCCTCTGCAAGGCGAGTTCACGACCTGG
AGCCCCTGGAGCCAGCCCCTGGCCTTCAGGACAAAGCCTGCAGC
CCTTGGGAAGGACACCATTCCGTGGCTCGGCCACCTCCTCGTGG
GTCTCAGCGGGGCTTTTGGCTTCATCATCTTAGTGTACTTGCTG
ATCAACTGCAGGAACACCGGGCCATGGCTGAAGAAGGTCCTGAA
GTGTAACACCCCAGACCCCTCGAAGTTCTTTTCCCAGCTGAGCT
CAGAGCATGGAGGAGACGTCCAGAAGTGGCTCTCTTCGCCCTTC
CCCTCATCGTCCTTCAGCCCTGGCGGCCTGGCACCTGAGATCTC
GCCACTAGAAGTGCTGGAGAGGGACAAGGTGACGCAGCTGCTCC
TGCAGCAGGACAAGGTGCCTGAGCCCGCATCCTTAAGCAGCAAC
CACTCGCTGACCAGCTGCTTCACCAACCAGGGTTACTTCTTCTT
CCACCTCCCGGATGCCTTGGAGATAGAGGCCTGCCAGGTGTACT
TTACTTACGACCCCTACTCAGAGGAAGACCCTGATGAGGGTGTG
GCCGGGGCACCCACAGGGTCTTCCCCCCAACCCCTGCAGCCTCT
GTCAGGGGAGGACGACGCCTACTGCACCTTCCCCTCCAGGGATG
ACCTGCTGCTCTTCTCCCCCAGTCTCCTCGGTGGCCCCAGCCCC
CCAAGCACTGCCCCTGGGGGCAGTGGGGCCGGTGAAGAGAGGAT
GCCCCCTTCTTTGCAAGAAAGAGTCCCCAGAGACTGGGACCCCC
AGCCCCTGGGGCCTCCCACCCCAGGAGTCCCAGACCTGGTGGAT
TTTCAGCCACCCCCTGAGCTGGTGCTGCGAGAGGCTGGGGAGGA
GGTCCCTGACGCTGGCCCCAGGGAGGGAGTCAGTTTCCCCTGGT
CCAGGCCTCCTGGGCAGGGGGAGTTCAGGGCCCTTAATGCTCGC
CTGCCCCTGAACACTGATGCCTACTTGTCCCTCCAAGAACTCCA
GGGTCAGGACCCAACTCACTTGGTGTAG

Parent Polypeptide: As used herein, the terms “parent polypeptide” or “parent protein” are used interchangeably to designate the source of a second polypeptide (e.g. a derivative or variant) which is modified with respect to a first “parent” polypeptide. In some instances, the parent polypeptide is a wild-type or naturally occurring form of a protein.

Percent Sequence Identity: “Percentage of sequence identity” or “percent sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Substantial identity of amino acid sequences normally means sequence identity of at least 40%. Percent identity of polypeptides can be any integer from 40% to 100%, for example, at least 40%, 45%, 50%, 55%, 60%, 65%, 70%, 75%, 80%, 85%, 90%, 95%, or 99%. In some embodiments, polypeptides that are “substantially similar” share sequences as noted above except that residue positions that are not identical may differ by conservative amino acid changes. Conservative amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acids substitution groups are: valine-leucine-isoleucine, phenylalanine-tyrosine, lysine-arginine, alanine-valine, aspartic acid-glutamic acid, and asparagine-glutamine.

Algorithms that are suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. Software for performing BLAST analyses is publicly available on the Web through the National Center for Biotechnology Information (at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, (1989)) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The term polypeptide include fusion proteins, including, but not limited to, fusion proteins with a heterologous amino acid sequence; fusion proteins with heterologous and homologous leader sequences; fusion proteins with or without N-terminal methionine residues; fusion proteins with amino acid sequences that facilitate purification such as chelating peptides; fusion proteins with immunologically tagged proteins; fusion proteins comprising a peptide with immunologically active polypeptide fragment (e.g. antigenic diphtheria or tetanus toxin or toxoid fragments) and the like.

Prevent: As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. The soluble form of hCD25 is an example of a soluble receptor that specifically binds hIL2. In some embodiments, the receptor is a cell surface receptor that comprises an extracellular domain (ECD) and a membrane associated domain which serves to anchor the ECD to the cell surface, in some instances in the absence of an intracellular domain or having a minimal intracellular domain which is not associated with intracellular signaling (e.g. hCD25). In some embodiments of cell surface receptors, the receptor is a membrane spanning polypeptide comprising an intracellular domain (ICD) and extracellular domain (ECD) operably linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a heteroxxmultimeric including heterodimeric (e.g. the intermediate affinity CD122/CD132 IL2 receptor), heterotrimeric (e.g. the high affinity CD25/CD122/CD132 hIL2 receptor) or homomultimeric (e.g., homodimeric, homotrimeric, or homotetrameric) complex that results in the activation of an intracellular signaling cascade (e.g. the Jak/STAT pathway) upon multimerization of the receptor components.

Recombinant: As used herein, the term “recombinant” is used as an adjective to refer to the method by which a polypeptide, nucleic acid, or cell was modified using recombinant DNA technology. A “recombinant protein” is a protein produced using recombinant DNA technology and is frequently abbreviated with a lower case “r” preceding the protein name to denote the method by which the protein was produced (e.g., recombinantly produced human growth hormone is commonly abbreviated “rhGH”). Similarly a cell is referred to as a “recombinant cell” if the cell has been modified by the incorporation (e.g. transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation) where the change is correlated with the activation, stimulation, or treatment, with or contact with exogenous agents or internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects. A “response” may be evaluated in vitro such as through the use of assay systems, surface plasmon resonance, enzymatic activity, mass spectroscopy, amino acid or protein sequencing technologies. A “response” may be evaluated in vivo quantitatively by evaluation of objective physiological parameters such as body temperature, bodyweight, tumor volume, blood pressure, results of X-ray or other imaging technology or qualitatively through changes in reported subjective feelings of well-being, depression, agitation, or pain. In some embodiments, the level of proliferation of CD3 activated primary human T-cells may be evaluated in a bioluminescent assay that generates a luminescent signal that is proportional to the amount of ATP present which is directly proportional to the number of cells present in culture as described in Crouch, et al. (1993) J. Immunol. Methods 160: 81-8 or through the use of commercially available assays such as the CellTiter-Glo® 2.0 Cell Viability Assay or CellTiter-Glo® 3D Cell Viability kits commercially available from Promega Corporation, Madison WI 53711 as catalog numbers G9241 and G9681 in substantial accordance with the instructions provided by the manufacturer. In some embodiments, the level of activation of T cells in response to the administration of a test agent may be determined by flow cytometric methods as described as determined by the level of STAT (e.g., STAT1, STAT3, STAT5) phosphorylation in accordance with methods well known in the art. For example, STATS phosphorylation may be measured using flow cytometric techniques as described in Horta, et al. supra., Garcia, et al., supra, or commercially available kits such as the Phospho-STATS (Tyr694) kit (commercially available from Perkin-Elmer, Waltham MA as Part Number 64AT5PEG) in performed in substantial accordance with the instructions provided by the manufacturer.

Selective: As used herein, the term “selective” or “selectively binds” is used to refer to a property of an agent to preferentially bind to and/or activate a particular cell type based on a certain property of a population of such cells. In some embodiments, the disclosure provides muteins that are CD25 selective in that such muteins display preferential activation of cells that expressing the orthogonal CD122 receptor relative to the cells expressing the wild-type CD122 receptor. Selectivity is typically assessed by activity measured in an assay characteristic of the activity induced in response to ligand/receptor binding. In some embodiments, IL2 orthologs of the present disclosure possess at least 3 fold, alternatively least 5 fold, alternatively at least 10 fold, alternatively at least 20 fold, alternatively at least 30 fold, alternatively at least 40 fold, alternatively at least 50 fold, alternatively at least 100 fold, alternatively at least 200 fold difference in EC50 on cells expressing the orthogonal CD122 receptor relative to the cells expressing the wild-type CD122 receptor as measured in the same assay. w

Significantly Reduced Binding: As used herein, the term “exhibits significantly reduced binding” is used with respect to a variant of a first molecule (e.g. a ligand) which exhibits a significant reduction in the affinity for a second molecule (e.g. receptor) relative the parent form of the first molecule. With respect to antibody variants (e.g an scFv molecule derived from a antibody), an antibody variant “exhibits significantly reduced binding” if the affinity of the variant antibody for an antigenic determinant of a molecule if the variant binds to such antigenic determinant and affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent antibody from which the variant antibody was derived. Similarly, with respect to variant ligands, a variant ligand “exhibits significantly reduced binding” if the affinity of the variant ligand binds to a receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent ligand from which the variant ligand was derived. Similarly, with respect to variant receptors, a variant receptor “exhibits significantly reduced binding” for a cognate ligand if the receptor binds a the cognate ligand with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the parent receptor from which the variant receptor was derived.

Specifically binds: As used herein the term “specifically binds” refers to the degree of selectivity or affinity for which one molecule binds to another. In the context of binding pairs (e.g., a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the equilibrium dissociation constant between antibody and to the second molecule of the binding pair is greater than about 10⁶M, alternatively greater than about 10⁸ M, alternatively greater than about 10¹⁰ M, alternatively greater than about 10¹¹ M, alternatively greater than about 10¹⁰ M, greater than about 10¹² M as determined by, e.g., Scatchard analysis (Munsen, et al. 1980 Analyt. Biochem. 107:220-239). In one embodiment where the ligand is an orthogonal IL2 and the receptor comprises an orthogonal CD122 ECD, the orthogonal IL2 specifically binds if the equilibrium dissociation constant of the IL2 ortholog/orthogonal CD122 ECD is greater than about 10⁵M, alternatively greater than about 10⁶ M, alternatively greater than about 10⁷M, alternatively greater than about 108M, alternatively greater than about 10⁹ M, alternatively greater than about 10¹⁰ M, or alternatively greater than about 10¹¹ M. Specific binding may be assessed using techniques known in the art including but not limited to competition ELISA, BIACORE® assays and/or KINEXA® assays.

Stem Cell: The term “stem cells” includes but is not limited to adult human stem cells, non-human embryonic stem cells, more particularly non-human stem cells, cord blood stem cells, progenitor cells, bone marrow stem cells, induced pluripotent stem cells, totipotent stein cells or hematopoietic stem cells. Representative human stem cells are CD34+ cells.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

Substantially Pure: As used herein, the term “substantially pure” indicates that a component of a composition makes up greater than about 50%, alternatively greater than about 60%, alternatively greater than about 70%, alternatively greater than about 80%, alternatively greater than about 90%, alternatively greater than about 95%, of the total content of the composition. A protein that is “substantially pure” comprises greater than about 50%, alternatively greater than about 60%, alternatively greater than about 70%, alternatively greater than about 80%, alternatively greater than about 90%, alternatively greater than about 95%, of the total content of the composition.

T Cell: As used herein the term “T-cell” or “T cell” is used in its conventional sense to refer to a lymphocyte that differentiates in the thymus, possess specific cell-surface antigen receptors, and include some that control the initiation or suppression of cell-mediated and humoral immunity and others that lyse antigen-bearing cells. In some embodiments the T cell includes without limitation naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, Tregs, inducible Tregs; memory T cells, e.g. central memory T cells, effector memory T cells, NKT cells, tumor infiltrating lymphocytes (TILs) and engineered variants of such T-cells including but not limited to CAR-T cells, recombinantly modified TILs and TCR engineered cells.

N-Terminus/C-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” refers to the position of a first amino acid residue relative to a second amino acid residue in a contiguous polypeptide sequence, the first amino acid being closer to the N-terminus of the polypeptide. “Immediately C-terminal” refers to the position of a first amino acid residue relative to a second amino acid residue in a contiguous polypeptide sequence, the first amino acid being closer to the C-terminus of the polypeptide.

Therapeutically Effective Amount: The phrase “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumors (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

Transmembrane Domain: The term “transmembrane domain” or “TM” refers to the domain of a membrane spanning polypeptide (e.g., a membrane spanning receptor polypeptide such as CD122, CD132 or a CAR) which, when the membrane spanning polypeptide is associated with a cell membrane, is embedded in the cell membrane and is in peptidyl linkage with the extracellular domain (ECD) and the intracellular domain (ICD) of a membrane spanning polypeptide. A transmembrane domain may be homologous (naturally associated with) or heterologous (not naturally associated with) with either or both of the extracellular and/or intracellular domains. A transmembrane domain may be homologous (naturally associated with) or heterologous (not naturally associated with) with either or both of the extracellular and/or intracellular domains. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived. Alternatively, the transmembrane domain of the receptor may be an artificial amino acid sequence which spans the plasma membrane. In some embodiments, where the receptor is chimeric receptor comprising the intracellular domain derived from a first parental receptor and a second extracellular domains are derived from a second different parental receptor, the transmembrane domain of the chimeric receptor is the transmembrane domain normally associated with either the ICD or the ECD of the parent receptor from which the chimeric receptor is derived.

Treat: The terms “treat”, “treating”, treatment” and the like refer to a course of action (such as administering IL2, a CAR-T cell, or a pharmaceutical composition comprising same) initiated with respect to a subject after a disease, disorder or condition, or a symptom thereof, has been diagnosed, observed, or the like in the subject so as to prevent, eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

Treg: The terms “regulatory T cell” or “Treg cell” as used herein refers to a type of CD4⁺ T cell that can suppress the responses of other T cells including but not limited to effector T cells (Teff). Treg cells are characterized by expression of CD4, the a-subunit of the IL2 receptor (CD25), and the transcription factor forkhead box P3 (FOXP3) (Sakaguchi, Annu Rev Immunol 22, 531-62 (2004). By “conventional CD4⁺ T cells” is meant CD4⁺ T cells other than regulatory T cells.

Variant: The terms “protein variant” or “variant protein” or “variant polypeptide” are used interchangeably herein to refer to a polypeptide that differs from a parent polypeptide by virtue of at least one amino acid modification. The parent polypeptide may be a naturally occurring or wild type (WT) polypeptide or may be a modified version of a WT polypeptide (i.e. mutein).

Wild Type: By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been modified by the hand of man.

### DETAILED DESCRIPTION OF THE INVENTION

### Introduction

The present disclosure provides methods and compositions that provide new opportunities for the applications of adoptive cell therapies including but not limited to chimeric antigen receptor (CAR) therapy.

### Variant/Mutein Nomenclature:

In some embodiments, the present disclosure provides variants of wild-type IL2 ligands and CD122 receptors comprising substitutions, deletions, and/or insertions relative to the wt hIL2 and wt hCD122 amino acid sequences, respectively. The residues which are modified in such variant protein may be designated herein by the one-letter or three-letter amino acid code followed by the position of such amino acid in the wild-type protein. For example, in the context of hIL2, “Cys125” or “C125” refers to the cysteine residue at position 125 of wt hIL2. The following nomenclature is used herein to refer to substitutions, deletions or insertions. Substitutions are designated herein by the one letter amino acid code for the wt hIL2 residue followed by the IL2 amino acid position followed by the single letter amino acid code for the new substituted amino acid. For example, “K35A” refers to a substitution of the lysine (K) residue at position 35 of the wt hIL2 sequence with an alanine (A) residue. A deletion is referred to as “des” followed by the amino acid residue and its position in wild-type molecule. For example the term “des-Ala1 hIL2” or “desA1 hIL2” refers to a human IL2 variant comprising a deletion of the alanine at position 1 of wt hIL2. The term “numbered in accordance with hIL2” as used herein refers to the identification of a location of particular amino acid with reference to the position at which that amino acid normally occurs in the mature sequence of the mature wild type hIL2. For example R81 refers to the eighty-first amino acid, arginine, that occurs in SEQ ID NO: 2. Similarly, the term numbered in accordance with hCD122 as used herein refers to the identification of a location of particular amino acid with reference to the position at which that amino acid normally occurs in the consensus sequence of the mature wild type hCD122 (SEQ ID NO: 1).

hoCD122^{pos}/wt hCD122^{neg} Cells

In one embodiment, the present disclosure provides an engineered human immune cell genomically modified to encode a hoCD122 or OCR operably linked to an expression control sequence to provide expression of an hoCD122 or OCR polypeptide in such engineered cell and wherein the engineered cell is genomically modified such that it does not express the native human CD122 receptor (a “hoCD122^{pos}/wt hCD122^{neg} cell”). In some embodiments, the hoCD122^{pos}/wt hCD122^{neg} cell is genomically modified by introduction of a polynucleotide encoding the hoCD122 or OCR is incorporated into the locus of the polynucleotide encoding the endogenous hCD122. In some embodiments, the hoCD122^{pos}/wt hCD122^{neg} cell is a T cell. In some embodiments, the hoCD122^{pos}/wt hCD122^{neg} cell is a NK cell. In some embodiments, the hoCD122^{pos}/wt hCD122^{neg} cell is a TIL. In some embodiments, the hoCD122^{pos}/wt hCD122^{neg} cell is a CAR-T cell.

In some embodiments, the present invention provides a method for the selective activation and/or proliferation of the engineered cell by contacting the hoCD122^{pos}/wt hCD122^{neg} cell with an hoIL2 in an amount sufficient to effect a change. As the ECD of the hoCD122 or OCR exhibits substantially reduced binding to wt hIL2 relative to hoIL2, elimination of the wt hCD122 from the hoCD122^{pos}/wt hCD122^{neg} cell enables selective activation and/or proliferation of the hoCD122^{pos}/wt hCD122^{neg} cells by contact with the hoIL2. Similarly, because naturally occurring cells do not express the hoCD122 receptor ECD, such naturally occurring cells are substantially non-responsive to hoCD122, expression of orthogonal CD122 is beneficial. In some embodiments, this is achieved by introducing an engineered hoCD122 coding sequence in place of (at the genetic location of) the endogenous human CD122 locus in human immune cells (e.g., including but not limited to lymphocyte or myeloid cells). In alternative embodiments, all alleles of the endogenous CD122 locus can be mutated or knocked out and the cell can be engineered to express an orthogonal CD122 protein.

As described herein, introduction of the orthogonal CD122 coding sequence in place of the endogenous CD122 coding sequence (or otherwise generating an human immune cell that expresses the orthogonal CD122 but does not express native CD122) allows for better control of expansion of such cells, for example by allowing specific expansion in response to orthogonal IL-2 and substantially reducing the responsiveness of the cells to wt hIL-2. An additional benefit is that when additional sequences (e.g., those encoding chimeric antigen receptors (CARs)) are co-introduced in the human immune cells (e.g., including but not limited to a lymphocyte or myeloid cells), such cells can be specifically expanded using an orthogonal IL-2, or when the additional sequences encode a CAR, a CAR ligand can be used alternatively or in combination with the orthogonal IL-2.

Endogenous CD122 refers to the CD122 naturally encoded in a human immune cell The coding sequence for the CD122 polypeptide including a signal peptide and associated natural expression control elements are included in the endogenous CD122 gene.

As described herein, by targeting insertion of a polynucleotide encoding the orthogonal CD122 into the endogenous CD122 gene locus, one can simultaneously disrupt the responsiveness of the cell to natural IL-2 while allowing for specific expansion of the cell in response to orthogonal IL2. This provides for specific control of expansion of the cell, separate from naturally-occurring cells, both in vitro or in vivo (or ex vivo). As discussed in more detail below, a human immune cell CD122 locus can be edited or partly or completely replaced with an orthogonal CD122 coding sequence, and optionally regulatory sequences to change the regulation of expression of the orthogonal CD122.

In some embodiments, the native human immune cell CD122 locus is edited to introduce sufficient changes (e.g., as discussed in detail below) in the native coding sequence such that the native CD122 promoter controls expression of the mutated native CD122 coding sequence, such that mutated native hCD122 is an hoCD122 polypeptide and the native polypeptide is not expressed.

hoCD122 Expression Control Sequences:

In some embodiments, part or all of the native hCD122 coding sequence can be replaced with an hoCD122 coding sequence. In some embodiments, as noted above, the hoCD122 expression will be under the control of the native hCD122 promoter and regulatory sequences, such that the hoCD122 is expressed substantially as the native CD122 would be expressed, i.e. in response to activation signals, cellular states and/or environmental conditions that would induce the expression of wtCD122.

In other embodiments, the native CD122 promoter or other regulatory sequences can be edited or replaced with different regulatory sequences such that the orthogonal CD122 is expressed differently than the native CD122 would be. Exemplary promoters that can be introduced to replace the native CD122 promoter include but are not limited to, e.g., Human ubiquitin C promoter (UbiC), SV40 early promoter (SV40), CMV immediate-early promoter (CMV), CAG promoter with CMV early enhancer (CAG(G)), or EFla promoter (EF1a).

### Genomic Modification:

In some embodiments, the hoCD122^{pos}/wt hCD122^{neg} cell is genomically modified by substitution of a portion of the nucleic acid sequence encoding the endogenous hCD122 so as to encode an hoCD122. The hoCD122 can produced by mutating residues of the endogenous CD122 coding (e.g., SEQ ID NO:1 or a sequence at least 95% identical to SEQ ID NO:1) such that they specifically bind to an orthogonal IL2 but do not specifically bind to a native IL2. See, e.g., U.S. Patent Publication No. US2019/0183933. In some embodiments, the binding affinity to the orthogonal IL2 is higher, e.g. 2×, 3×, 4×, 5×, 10× or more of the affinity of the native IL2 for the native CD122. In some embodiments, the affinity of the orthogonal IL2 for the cognate orthogonal CD122 exhibits affinity comparable to the affinity of the native IL2 for the native CD122, e.g. having an affinity that is least about 1% of the binding affinity of the native CD122 for the native IL2, at least about 5%, at least about 10%, at least about 25%, at least about 50%, at least about 75%, at least about 100%. In some cases, the orthogonal CD122 is modified at one or more residues selected from R41, R42, Q70, K71, T73, T74, V75, S132, H133, Y134, F135, E136, and Q214 relative to native human CD122 (e.g., compared to SEQ ID NO:1). In some embodiments, the hoCD122 is modified at H133 and Y134. In some embodiments, the hoCD122 comprises substitutions of H133D and Y134F. In some embodiments, the hoCD122 is substituted at Q70, T73, H133, Y134 in the native human CD122 protein. In some embodiments, hoCD122 comprises amino acid substitutions H133 and Y134. In some embodiments, the amino acid substitution is to an acidic amino acid, e.g. aspartic acid and/or glutamic acid. Specific amino acid substitutions include, without limitation, Q70Y; T73D; T73Y; H133D, H133E; H133K; Y134F; Y134E; Y134R relative to the native human CD122. The selection of an orthologous cytokine may vary with the choice of orthologous receptor.

The preparation of lymphocytes (e.g., T cells) or myeloid cells useful in the practice of the present disclosure can be achieved by introducing a targeted cleavage site within the endogenous CD122 gene coding sequence and, for example, introducing a homology dependent repair (HDR) template nucleic acid, preferable having one or two flanking homology arms to improve efficiently of introduction of the HDR template at the cleavage site, thereby inducing replacement of the endogenous CD122 coding sequence or a portion thereof with a coding sequence or portion thereof for the orthogonal CD122 coding sequence. Thus the orthogonal CD122 coding sequence is in the place of the endogenous CD122 in the cell genome, replacing the endogenous sequence. Optimally, both alleles of the endogenous CD122 genes in the genome are replaced with the orthogonal CD122, such that the resulting cell does not express a native (endogenous) CD122 protein. The entire coding sequence of the endogenous CD122 can be replaced, or one or more portion of the endogenous coding sequence can be modified in this manner to allow for expression from the native CD122 promoter or from a promoter that is also introduced.

Targeted cleavage within the endogenous CD122 gene coding sequence (e.g., for insertion of orthogonal CD122 in its place or for mutating or knocking out endogenous CD122) can be introduced using any number of guided (targeted) nucleases. A “guided nuclease” refers to a DNA nuclease that is targeted to a particular genomic DNA sequence, for example by a separate small guide RNA (sgRNA) or a fused protein sequence that targets the DNA sequence. Any method of delivery can be used to deliver the nuclease and guide molecule if separate from the nuclease. In some embodiments, the nuclease and a guide RNA are delivered by the same mechanism. In some embodiments, the nuclease is delivered to the T-cell by one mechanism (e.g., as a protein or encoded by a nucleic acid) and the sgRNA is delivered to the T-cell by a second mechanism.

Any method of genetic manipulation can be used to introduce the orthogonal CD122 coding sequence or mutations that change the endogenous CD122 coding sequence to encode the orthogonal CD122. In some embodiments, a double-strand break (DSB) or nick for can be created by a site-specific nuclease in or near the endogenous CD122 gene. Exemplary targeted nucleases include but are not limited to zinc-finger nuclease (ZFN) or TAL effector domain nuclease (TALEN), or the CRISPR/Cas9 system with an engineered crRNA/tract RNA (single guide RNA) to guide specific cleavage. See, for example, Burgess (2013) Nature Reviews Genetics 14:80-81, Urnov et al. (2010) Nature 435(7042):646-51; United States Patent Publications 20030232410; 20050208489; 20050026157; 20050064474; 20060188987; 20090263900; 20090117617; 20100047805; 20110207221; 20110301073 20110301073; 20130177983; 20130177960 and International Publication WO 2007/014275, WO2003087341; WO2000041566; WO2003080809. Nucleases specific for targeted genes can be utilized such that a transgene construct is inserted by either homology directed repair (HDR) or by end capture during non-homologous end joining (NHEJ) driven processes.

“Homology directed repair” or HDR refers to a cellular process in which cut or nicked ends of a DNA strand are repaired by polymerization from a homologous template nucleic acid. Thus, the original sequence is replaced with the sequence of the template. An exogenous template nucleic acid (i.e., an “HDR template”) can be introduced to obtain a specific HDR-induced change of the sequence at the target site. In this way, specific mutations can be introduced at the cut site. A single-stranded DNA template or a double-stranded DNA template can be used by a cell as a template for editing the genome of a lymphocyte or myeloid cell, for example, by HDR. Generally, the single-stranded DNA template or a double-stranded DNA template has at least one region of homology to a target site. In some cases, the single-stranded DNA template or double-stranded DNA template has two homologous regions, for example, a 5′ end and a 3′ end, flanking a region that contains a heterologous sequence to be inserted at a target cut or insertion site. The HDR template can be introduced with the guided nuclease and a guide RNA or DNA or can be introduced into the target cell separately. The coding sequence of the orthogonal CD122 in the HDR template can be modified with one or more mutations such that PAM sites are eliminated. In some embodiments, these mutations are selected such that there is no change in amino acid encoded (silent mutation).

In some embodiments, the HDR template comprises coding sequence of the orthogonal CD122 as well as at least one additional coding sequence. In some embodiments, the coding sequence of the orthogonal CD122 and the one or more additional coding sequence are linked to encode a fusion protein, wherein the coding sequence of the orthogonal CD122 and the addition coding sequence are separated by a self-cleaving peptide. In some embodiments, the self-cleaving peptide, e.g., such as a P2A, E2A, F2A or T2A peptide. In some embodiments, the addition coding sequence encodes a CAR (e.g., as described above.

Any nuclease that can be targeted to a particular genome sequence to induce sequence-specific cleavage and thus allow for targeted mutagenesis can be used. Exemplary nucleases include, for example, TALE nucleases (TALENs), zinc-finger proteins (ZFPs), zinc-finger nucleases (ZFNs), DNA-guided polypeptides such as Natronobacterium gregoryi Argonaute (NgAgo), and CRISPR/Cas RNA-guided polypeptides including but not limited to Cas9, CasX, CasY, Cpf1, Cms1, MAD7 and the like.

Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known. For example, the amino acid sequence of S. pyogenes Cas9 protein may be found in the SwissProt database under accession number Q99ZW2. In some embodiments, the CRISPR enzyme has DNA cleavage activity, such as Cas9. In some embodiments the CRISPR enzyme is Cas9, and may be Cas9 from S. Pyogenes, S. aureus or S. pneumonia or Actinobacteria, Aquificae, Bacteroidetes-Chlorobi, Chlamydiae-Verrucomicrobia, Chlroflexi, Cvanobacteria, Firmicutes, Proteobacteria, Spirochaetes, or Thermotogae. In some embodiments, the CRISPR enzyme directs cleavage of one or both strands at the location of a target sequence, such as within the target sequence and/or within the complement of the target sequence. In some embodiments, two single stranded nicks are made on opposition strands within a short span of DNA (e.g., within 1 kb in some embodiments).

In some embodiments, the endogenous CD122 locus in a human immune cell is mutated or knocked out such that no alleles of endogenous CD122 are expressed or at least such that the cell is substantially non-responsive to native IL-2. In some embodiments, cells can be selected for double knockouts (e.g., both alleles of a diploid cell not expressing native CD122) via sorting (e.g., FACS) methods. The resulting cells can be then engineered to express orthogonal CD122 in any way desired. Merely as examples, in so embodiments, an orthogonal CD122 coding sequence or expression cassette can be introduced into the cell by lentivirus/retrovirus or any other integrating method, including but not limited to by sleeping beauty transposons (see, e.g., Ivics, Gene Therapy (2020)). Various vectors are known in the art and can be used for this purpose, e.g., viral vectors, plasmid vectors, minicircle vectors. Expression vectors can contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media. Alternatively, or in combination, an orthogonal IL2 may be employed in methods of selectively expanding such engineered T cells (e.g., human T-cells) which have been engineered to express a corresponding modified human CD122 (e.g., an orthogonal CD122).

IL2 orthologs may be employed as described above in methods of selectively expanding such engineered T cells (e.g., human T-cells) which have been engineered to express a corresponding orthogonal CD122 receptor. T-cells useful for engineering with the constructs described herein include naïve T-cells, central memory T-cells, effector memory T-cells or combination thereof. T cells for engineering as described above are collected from a subject or a donor may be separated from a mixture of cells by techniques that enrich for desired cells or may be engineered and cultured without separation. Alternatively, the T cells for engineering may be separated from other cells. Techniques providing accurate separation include fluorescence activated cell sorters. The cells may be selected against dead cells by employing dyes associated with dead cells (e.g., propidium iodide). The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification or may be frozen at liquid nitrogen temperatures and stored, being thawed and capable of being reused. The cells will usually be stored in 10% DMSO, 50% FCS, 40% RPMI 1640 medium.

T-cells useful for engineering as described herein include but are not limited to naïve T-cells, central memory T-cells, effector memory T-cells, regulatory CD4+ T cells, natural killer T-cells, or combination thereof. In some embodiments, the cells comprise a ratio of CD8+ and CD4+ cells (see, e.g., Turtle, et al, J Clin Invest. 2016; 126(6):2123-2138). In some embodiments, the ratio is within 20-80 CD4+ cells:20-80 CD8+ cells, e.g., 20:80, 30:70, 40:60, 50:50, 60:40, 70:30, or 80:20 CD4+:CD8+ cells. T cells for engineering as described above are collected from a subject or a donor may be separated from a mixture of cells by techniques that enrich for desired cells or may be engineered and cultured without separation. Alternatively, the T cells for engineering may be separated from other cells. Techniques providing accurate separation include fluorescence activated cell sorters. The cells may be selected against dead cells by employing dyes associated with dead cells (e.g., propidium iodide). The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS). The collected and optionally enriched cell population may be used immediately for genetic modification or may be frozen at liquid nitrogen temperatures and stored, being thawed and capable of being reused. The cells will usually be stored in 10% DMSO, 50% FCS, 40% RPMI 1640 medium. In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Seminars in Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

The hoCD122^{pos}/wt hCD122^{neg} cells are capable of selective modulation (e.g. activation and/or proliferation) in response to contacting the hoCD122^{pos}/wt hCD122^{neg} cell with a biologically effective amount of a orthogonal ligand wherein said orthogonal ligand specifically binds to the ECD of the hoCD122 or OCR of the hoCD122^{pos}/wt hCD122^{neg} cell. In some embodiments, the orthogonal ligand of the following formula.

Orthogonal hIL2 (hoIL2s)s:

In various embodiments, the compositions and methods of the present disclosure comprise the use of human IL2 orthologs (i.e., orthogonal hIL-2, hoIL2) which are hIL2 muteins comprising an amino acid sequence of the following formula (SEQ ID NO: 15): (AA1)-(AA2)-(AA3)-(AA4)-(AA5)-(AA6)-(AA7)-(AA8)-(AA9)-T10-Q11-L12-(AA13)-(AA14)-(AA15)-(AA16)-L17-(AA18)-(AA19)-(AA20)-L21-(AA22)-(AA23)424-L25-N26-(AA27)428-N29-N30-Y31-K32-N33-P34-K35-L36-T37-(AA38)-(AA39)-L40-T41-(AA42)-K43-F44-Y45-M46-P47-K48-K49-A50-(AA51)-E52-L53-K54-(AA55)-L56-Q57-058-L59-E60-E61-E62-L63-K64-P65-L66-E67-E68-V69-L70-N71-L72-A73-(AA74)-S75-K76-N77-F78-H79-(AA80-(AA81)-P82-R83-D84-(AA85)-(AA86)-S87-(AA88)-(AA89)-N90-(AA91)-(AA92)-V93-L94-E95-L96-(AA97)-G98-S99-E100-T101-T102-F103-(AA104)-C105-E106-Y107-A108-(AA109)-E110-T111-A112-(AA113)-I114-V115-E116-F117-L118-N119-R120-W121-I122-T123-F124-(AA125)-(AA126)-S127-I128-I129-(AA130)-T131-L132-T133

wherein:
AA1 is A (wild type) or deleted;
AA2 is P (wild type) or deleted;
AA3 is T (wild type), C, A, G, Q, E, N, D, R, K, P, or deleted
AA4 is S (wild type) or deleted;
AA5 is S (wild type) or deleted;
AA6 is S (wild type) or deleted;
AA7 is T (wild type) or deleted;
AA8 is K (wild type) or deleted;
AA9 is K (wild type) or deleted;
AA13 is Q (wild type), W or deleted;
AA14 is L (wild type), M, W or deleted;
AA15 is E (wildtype), K, D, T, A, S, Q, H or deleted;
AA16 is H (wildtype), N or Q or deleted;
AA18 is L (wild type) or R, G, M, F, E, H, W, K, Q, S, V, I, Y, H, D or T;
AA19 is L (wildtype), A, V, I or deleted;
AA20 is D (wildtype), T, S M L, or deleted;
AA22 is Q (wild type) or F, E, G, A, L, M, F, W, K, S, V, I, Y, H, R, N, D, T, F or deleted
AA23 is M (wild type), A, W, H, Y, F, Q, S, V, L, T, or deleted;
AA27 IS G (wildtype), K, S or deleted;
AA38 is R (wild type), W or G;
AA39 is M (wildtype), L or V;
AA42 is F (wildtype) or K;
AA51 is T (wildtype), I or deleted
AA55 is H (wildtype) or Y;
AA74 is Q (wild type), N, H, S;
AA80 is L (wild type), F or V;
AA81 is R (wild type), I, D, Y, T or deleted
AA85 is L (wild type) or V;
AA86 is I (wild type) or V;
AA88 is N (wildtype), E or Q or deleted;
AA89 is I (wild type) or V;
AA91 is V (wild type), R or K;
AA92 is I (wild type) or F;
AA97 is K (wild type) or Q;
AA104 is M (wild type) or A;
AA109 is D (wildtype), C or a non-natural amino acid with an activated side chain;
AA113 is T (wild type) or N;
AA125 is C (wild type), A or S;
AA126 is Q (wild type) or H, M, K, C, D, E, G, I, R, S, or T; and/or
AA130 is S (wild type), T or R.

In some embodiments, the present disclosure provides hIL2 orthologs which are hIL2 polypeptides comprising the following sets of amino acid modifications numbered in accordance with wild-type hIL-2:
[E15S-H16Q-L19V-D20L-Q22K]
[H16N, L19V, D20N, Q22T, M23H, G27K];
[E15D, H16N, L19V, D20L, Q22T, M23H];
[E15D, H16N, L19V, D20L, Q22T, M23A],
[E15D, H16N, L19V, D20L, Q22K, M23A];
[E15S; H16Q; L19V, D20T; Q22K, M23L];
[E15S; H16Q; L19V, D20T; Q22K, M23S];
[E15S; H16Q; L19V, D20S; Q22K, M23S];
[E15S; H16Q; L191, D20S; Q22K; M23L];
[E15S; L19V; D20M; Q22K; M23S];
[E15T; H16Q; L19V; D20S; M23S];
[E15Q; L19V; D20M; Q22K; M23S];
[E15Q; H16Q; L19V; D20T; Q22K; M23V];
[E15H; H16Q; L191; D20S; Q22K; M23L];
[E15H; H16Q; L191; D20L; Q22K; M23T];
[L19V; D20M; Q22N; M23S].

In some embodiments, the hoIL2 is a polypeptide as described in Garcia, et al. U.S. Pat. No. 10,869,887B2 issued Dec. 22, 2020, the entire teaching of which is herein incorporated by reference.

### Conservative Amino Acid Substitutions

in addition to the foregoing modifications that contribute to the activity and selectivity of the IL2 ortholog for the CD122 orthogonal receptor, the IL2 ortholog may comprise one or more modifications to its primary structure that provide minimal effects on the activity IL2. In some embodiments, the IL2 orthologs of the present disclosure may further comprise one more conservative amino acid substitution within the wild type IL-2 amino acid sequence. Such conservative substitutions include those described by Dayhoff in The Atlas of Protein Sequence and Structure 5 (1978), and by Argos in EMBO J., 8:779-785 (1989). Conservative substitutions are generally made in accordance with the following chart depicted as Table 3.

TABLE 3
Exemplary Conservative Amino Acid Substitutions

| Wild type Residue | Substitution(s) |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu, Met, Leu, Ile |
| Phe | Met, Leu, Tyr, Trp |
| Ser | Thr |
| Thr | Ser |
| Trp | Tyr, Phe |
| Tyr | Trp, Phe |
| Val | Ile, Leu |

Substantial changes in function or immunological identity may be made by selecting amino acid substitutions that are less conservative than those indicated in Table 3. For example substitutions may be made which more significantly affect the structure of the polypeptide backbone or disrupt secondary or tertiary elements including the substitution of an amino acid with a small uncharged side chain (e.g. glycine) with a large charge bulky side chain (asparagine). In particular, substitution of those IL2 residues which are involved in the amino acids that interact with one or more of CD25, CD122 and/or CD123 as may be discerned from the crystal structure of IL2 in association with its receptors as described in

In addition to the foregoing modifications that contribute to the activity and selectivity of the IL2 ortholog for the CD122 orthogonal receptor, the IL2 ortholog may comprise one or more modifications to its primary structure. Modifications to the primary structure as provided above may optionally further comprise modifications do not substantially diminish IL2 activity of the IL2 ortholog including but not limited to the substitutions: N30E; K32E; N33D; P34G; T37I, M39Q, F42Y, F44Y, P47G, T51I, E52K, L53N, Q57E, M104A (see U.S. Pat. No. 5,206,344).

### Removal of Glycosylation Site

The IL2 orthologs of the present disclosure may comprises comprise modifications to eliminate the O-glycosylation site at position Thr3 to facilitate the production of an aglycosylated IL2 ortholog when the IL2 ortholog expressed in mammalian cells such as CHO or HEK cells. Thus, in certain embodiments the IL2 ortholog comprise a modification which eliminates the O-glycosylation site of IL-2 at a position corresponding to residue 3 of human IL-2. In one embodiment said modification which eliminates the O-glycosylation site of IL-2 at a position corresponding to residue 3 of human IL-2 is an amino acid substitution. Exemplary amino acid substitutions include T3A, T3G, T3Q, T3E, T3N, T3D, T3R, T3K, and T3P which removes the glycosylation site at position 3 without eliminating biological activity (see U.S. Pat. No. 5,116,943; Weiger et al., (1989) Eur. J. Biochem., 180:295-300). In a specific embodiment, said modification is the amino acid substitution T3A.

### N Terminal Deletions:

When produced recombinantly in bacterial expression systems directly in the absence of a leader sequence, endogenous proteases result in the deletion of the N-terminal Met-Ala1 residues to provide “desAla1” IL2 orthologs. IL2 orthologs may comprise deletion of the first two amino acids (desAla1-desPro2) as well as substitution of the Thr3 glycosylation with a cysteine residue (T3C) to facilitate for N-terminal modification, especially PEGylation of the sulfhydryl group of the cysteine (See, e.g. Katre, et al. U.S. Pat. No. 5,206,344 issued Apr. 27, 1993). The IL2 orthologs may further comprise elimination of N-terminal amino acids at one or more of positions 1-9, alternatively positions 1-8, alternatively positions 1-7 alternatively positions 1-6, alternatively positions 1-5, alternatively positions 1-4, alternatively positions 1-3, alternatively positions 1-2.

### Modifications to Minimize Vascular Leak Syndrome

In some embodiments of the disclosure, the IL2 ortholog comprises amino acid substitutions to avoid vascular leak syndrome, a substantial negative and dose limiting side effect of the use of IL2 therapy in human beings without out substantial loss of efficacy. See, Epstein, et al., U.S. Pat. No. 7,514,073B2 issued Apr. 7, 2009. Examples of such modifications which are included in the IL2 orthologs of the present disclosure include one or more of R38W, R38G, R39L, R39V, F42K, and H55Y.

### Modifications to Extend Duration of Action In Vivo

As discussed above, the compositions of the present disclosure include IL2 orthologs that have been modified to provide for an extended lifetime in vivo and/or extended duration of action in a subject. Such modifications to provided extended lifetime and/or duration of action include modifications to the primary sequence of the IL2 ortholog, conjugation to carrier molecules, (e.g. albumin, acylation, PEGylation), and Fc fusions.

Sequence Modifications to Extend Duration of Action In Vivo

As discussed above, the term IL2 ortholog includes modifications of the IL2 ortholog to provide for an extended lifetime in vivo and/or extended duration of action in a subject.

In some embodiments, the IL2 ortholog may comprise certain amino acid substitutions that result in prolonged in vivo lifetime. For example, Dakshinamurthi, et al. (International Journal of Bioinformatics Research (2009) 1(2):4-13) state that one or more of the substitutions in the IL2 polypeptide V91R, K97E and T113N will result in an IL2 variant possessing enhanced stability and activity. In some embodiments, the IL2 orthologs of the present disclosure comprise one, two or all three of the V91R, K97E and T113N modifications.

Conjugates and Carrier Molecules

In some embodiments the IL2 ortholog is modified to provide certain properties to the IL2 ortholog (e.g. extended duration of action in a subject) which may be achieve through conjugation to carrier molecules to provide desired pharmacological properties such as extended half-life. In some embodiments, the IL2 ortholog can be covalently linked to the Fc domain of IgG, albumin, or other molecules to extend its half-life, e.g. by PEGylation, glycosylation, fatty acid acylation, and the like as known in the art.

Albumin Fusions

In some embodiments, the IL2 ortholog is expressed as a fusion protein with an albumin molecule (e.g. human serum albumin) which is known in the art to facilitate extended exposure in vivo.

In one embodiment of the invention, the hIL2 ortholog is conjugated to albumin referred to herein as an “IL2 ortholog albumin fusion.” The term “albumin” as used in the context hIL2 ortholog albumin fusions include albumins such as human serum albumin (HSA), cyno serum albumin, and bovine serum albumin (BSA). In some embodiments, the HSA the HSA comprises a C34S or K573P amino acid substitution relative to the wild type HSA sequence According to the present disclosure, albumin can be conjugated to a hIL2 ortholog at the carboxyl terminus, the amino terminus, both the carboxyl and amino termini, and internally (see, e.g., U.S. Pat. Nos. 5,876,969 and 7,056,701). In the HSA-hIL2 ortholog polypeptide conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising a hIL2 ortholog polypeptide fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is effected by a linker such as a peptide linker or modified version thereof as more fully discussed below.

Alternatively, the hIL2 ortholog albumin fusion comprises IL2 orthologs that are fusion proteins which comprise an albumin binding domain (ABD) polypeptide sequence and an IL2 ortholog polypeptide. As alluded to above, fusion proteins which comprise an albumin binding domain (ABD) polypeptide sequence and an hIL2 ortholog polypeptide can, for example, be achieved by genetic manipulation, such that the nucleic acid coding for HSA, or a fragment thereof, is joined to the nucleic acid coding for the one or more IL2 ortholog sequences. In some embodiments, the albumin-binding peptide comprises the amino acid sequence DICLPRWGCLW (SEQ ID NO: 14).

The IL2 ortholog polypeptide can also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria, dendritic cells, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen Such conjugated forms, if desired, can be used to produce antibodies against a polypeptide of the present disclosure.

In some embodiments, the IL2 ortholog is conjugated (either chemically or as a fusion protein) with an XTEN which provides extended duration of akin to PEGylation and may be produced as a recombinant fusion protein in E. coli. XTEN polymers suitable for use in conjunction with the IL2 orthologs of the present disclosure are provided in Podust, et al. (2016) “Extension of in vivo half-life of biologically active molecules by XTEN protein polymers”, J Controlled Release 240:52-66 and Haeckel et al. (2016) “XTEN as Biological Alternative to PEGylation Allows Complete Expression of a Protease—Activatable Killin-Based Cytostatic” PLOS ONE|DOI:10.1371/journal.pone.0157193 Jun. 13, 2016. The XTEN polymer may fusion protein may incorporate a protease sensitive cleavage site between the XTEN polypeptide and the IL2 ortholog such as an MMP-2 cleavage site.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

In some embodiments, the IL-2 mutein also may be linked to additional therapeutic agents including therapeutic compounds such as anti-inflammatory compounds or antineoplastic agents, therapeutic antibodies (e.g. Herceptin), immune checkpoint modulators, immune checkpoint inhibitors (e.g. anti-PD1 antibodies), cancer vaccines as described elsewhere in this disclosure. Anti-microbial agents include aminoglycosides including gentamicin, antiviral compounds such as rifampicin, 3′-azido-3′-deoxythymidine (AZT) and acylovir, antifungal agents such as azoles including fluconazole, plyre macrolides such as amphotericin B, and candicidin, anti-parasitic compounds such as antimonials, and the like. The IL2 ortholog may be conjugated to additional cytokines as CSF, GSF, GMCSF, TNF, erythropoietin, immunomodulators or cytokines such as the interferons or interleukins, a neuropeptide, reproductive hormones such as HGH, FSH, or LH, thyroid hormone, neurotransmitters such as acetylcholine, hormone receptors such as the estrogen receptor. Also included are non-steroidal anti-inflammatories such as indomethacin, salicylic acid acetate, ibuprofen, sulindac, piroxicam, and naproxen, and anesthetics or analgesics. Also included are radioisotopes such as those useful for imaging as well as for therapy.

The IL2 orthologs of the present disclosure may be chemically conjugated to such carrier molecules using well known chemical conjugation methods. Bi-functional cross-linking reagents such as homofunctional and heterofunctional cross-linking reagents well known in the art can be used for this purpose. The type of cross-linking reagent to use depends on the nature of the molecule to be coupled to IL-2 mutein and can readily be identified by those skilled in the art. Alternatively, or in addition, the IL2 ortholog and/or the molecule to which it is intended to be conjugated may be chemically derivatized such that the two can be conjugated in a separate reaction as is also well known in the art.

PEGylation:

In some embodiments, the IL2 ortholog is conjugated to one or more water-soluble polymers. Examples of water soluble polymers useful in the practice of the present invention include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol, polysaccharides, poly-alpha-hydroxy acid, polyvinyl alcohol (PVA), polyphosphazene, polyoxazolines (POZ), poly(N-acryloylmorpholine), or a combination thereof.

In some embodiments the IL2 ortholog is conjugated to one or more polyethylene glycol molecules or “PEGylated.” Although the method or site of PEG attachment to IL2 ortholog may vary, in certain embodiments the PEGylation does not alter, or only minimally alters, the activity of the IL2 ortholog.

In some embodiments, a cysteine may be substituted for the threonine at position 3 (3TC) to facilitate N-terminal PEGylation using particular chemistries.

In some embodiments, selective PEGylation of the IL2 ortholog (for example by the incorporation of non-natural amino acids having side chains to facilitate selective PEG conjugation chemistries as described Ptacin, et al., (PCT International Application No. PCT/US2018/045257 filed Aug. 3, 2018 and published Feb. 7, 2019 as International Publication Number WO 2019/028419A1 may be employed to generate an IL2 ortholog with having reduced affinity for one or more subunits (e.g. CD25, CD132) of an IL2 receptor complex. For example, an hIL2 ortholog incorporating non-natural amino acids having a PEGylatable specific moiety at those sequences or residues of IL2 identified as interacting with CD25 including amino acids 34-45, 61-72 and 105-109 typically provides an IL2 ortholog having diminished binding to CD25. Similarly, an hIL2 ortholog incorporating non-natural amino acids having a PEGylatable specific moiety at those sequences or residues of IL2 identified as interacting with hCD132 including amino acids 18, 22, 109, 126, or from 119-133 provides an IL2 ortholog having diminished binding to hCD132.

In certain embodiments, the increase in half-life is greater than any decrease in biological activity. PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the PEG-IL2 ortholog can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, alternatively about 30,000 to about 40,000 daltons. In one embodiment of the invention, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

The PEG can be bound to an IL2 ortholog of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of IL2 orthologs is facilitated by the incorporation of non-natural amino acids bearing unique side chains to facilitate site specific PEGylation. The incorporation of non-natural amino acids into polypeptides to provide functional moieties to achieve site specific pegylation of such polypeptides is known in the art. See e.g. Ptacin, et al., (PCT International Application No. PCT/US2018/045257 filed Aug. 3, 2018 and published Feb. 7, 2019 as International Publication Number WO 2019/028419A1. In one embodiment, the IL2 orthologs of the present invention incorporate a non-natural amino acid at position D109 of the IL2 ortholog. In one embodiment of the invention the IL2 ortholog is a PEGylated at position 109 of the IL2 ortholog to a PEG molecule having a molecular weight of about 20 kD, alternatively about 30 kD, alternatively about 40 kD.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present invention include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g. Sunbright® ME-200AL, NOF, a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NETS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

As previously noted, the PEG may be attached directly to the IL2 ortholog or via a linker molecule. Suitable linkers include “flexible linkers” which are generally of sufficient length to permit some movement between the modified polypeptide sequences and the linked components and molecules. The linker molecules are generally about 6-50 atoms long. The linker molecules can also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids. Examples of flexible linkers include glycine polymers (G)ₙ, glycine-serine polymers, glycine-alanine polymers, alanine-serine polymers, and other flexible linkers. Glycine and glycine-serine polymers are relatively unstructured, and therefore can serve as a neutral tether between components. Further examples of flexible linkers include glycine polymers (G)ₙ, glycine-alanine polymers, alanine-serine polymers, glycine-serine polymers. Glycine and glycine-serine polymers are relatively unstructured, and therefore may serve as a neutral tether between components. A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein.

Further, such linkers may be used to link the IL2 ortholog to additional heterologous polypeptide components as described herein, the heterologous amino acid sequence may be a signal sequence and/or a fusion partner, such as, albumin, Fc sequence, and the like.

In one embodiment of the disclosure, the IL2 ortholog is a human IL2 ortholog of the structure:
[PEG]-[linker]ₙ-[hoIL2]
   wherein n=0 or 1, or
[PEG]-[linker]ₙ-hIL2[desAla1E15S-H16Q-L19V-D20L-Q22K-M23A]
   wherein n=0 or 1, or
In another embodiment of the invention, the IL2 ortholog is a human IL2 ortholog of the structure

(SEQ ID NO: 13)
40kD-PEG-(linker)ₙ-PTSSSTKKTQLQLSQLLVLLKAILN
GINNYKNPKLTRMLTFKFYMPKKATELKHLQCLEEELKPLEEV
LNLAQSKNFHLRPRDLISNINVIVLELKGSETTFMCEYADETA
TIVEFLNRWITFCQSIISTLT

wherein n=0 or 1. In one embodiment, the IL2 ortholog is a human IL2 ortholog having the structure: 40 kD branched PEG-linker-hIL2[desAla1-E15S-H16Q-L19V-D20L-Q22K-M23A]-COOH, wherein 40 kD branched PEG-linker is of the structure:

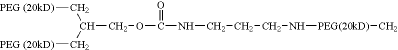

Acylation

In some embodiments, the IL2 ortholog of the present disclosure may be acylated by conjugation to a fatty acid molecule as described in Resh (2016) Progress in Lipid Research 63: 120-131. Examples of fatty acids that may be conjugated include myristate, palmitate and palmitoleic acid. Myristoylate is typically linked to an N-terminal glycine but lysines may also be myristoylated. Palmitoylation is typically achieved by enzymatic modification of free cysteine—SH groups such as DHHC proteins (SEQ ID NO: 16) catalyze S-palmitoylation. Palmitoleylation of serine and threonine residues is typically achieved enzymatically using PORCN enzymes.

### Acetylation

In some embodiments, the IL-2 mutein is acetylated at the N-terminus by enzymatic reaction with N-terminal acetyltransferase and, for example, acetyl CoA. Alternatively, or in addition to N-terminal acetylation, the IL-2 mutein is acetylated at one or more lysine residues, e.g. by enzymatic reaction with a lysine acetyltransferase. See, for example Choudhary et al. (2009) Science 325 (5942):834L2 ortho840.

Fc Fusions

In some embodiments, the IL2 fusion protein may incorporate an Fc region derived from the IgG subclass of antibodies that lacks the IgG heavy chain variable region. The “Fc region” can be a naturally occurring or synthetic polypeptide that is homologous to the IgG C-terminal domain produced by digestion of IgG with papain. IgG Fc has a molecular weight of approximately 50 kDa. The mutant IL-2 polypeptides can include the entire Fc region, or a smaller portion that retains the ability to extend the circulating half-life of a chimeric polypeptide of which it is a part. In addition, full-length or fragmented Fc regions can be variants of the wild type molecule. That is, they can contain mutations that may or may not affect the function of the polypeptides; as described further below, native activity is not necessary or desired in all cases. In certain embodiments, the IL-2 mutein fusion protein (e.g., an IL-2 partial agonist or antagonist as described herein) includes an IgG1, IgG2, IgG3, or IgG4 Fc region. Exemplary Fc regions can include a mutation that inhibits complement fixation and Fc receptor binding, or it may be lytic, i.e., able to bind complement or to lyse cells via another mechanism such as antibody-dependent complement lysis (ADCC).

In some embodiments, the IL2 ortholog comprises a functional domain of an Fc-fusion chimeric polypeptide molecule. Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates. The “Fc region” useful in the preparation of Fc fusions can be a naturally occurring or synthetic polypeptide that is homologous to an IgG C-terminal domain produced by digestion of IgG with papain. IgG Fc has a molecular weight of approximately 50 kDa. The IL2 orthologs may provide the entire Fc region, or a smaller portion that retains the ability to extend the circulating half-life of a chimeric polypeptide of which it is a part. In addition, full-length or fragmented Fc regions can be variants of the wild type molecule. In a typical presentation, each monomer of the dimeric Fc carries a heterologous polypeptide, the heterologous polypeptides being the same or different.

In some embodiments, when the IL2 ortholog is to be administered in the format of an Fc fusion, particularly in those situations when the polypeptide chains conjugated to each subunit of the Fc dimer are different, the Fc fusion may be engineered to possess a “knob-into-hole modification.” The knob-into-hole modification is more fully described in Ridgway, et al. (1996) Protein Engineering 9(7):617-621 and U.S. Pat. No. 5,731,168, issued Mar. 24, 1998. The knob-into-hole modification refers to a modification at the interface between two immunoglobulin heavy chains in the CH₃ domain, wherein: i) in a CH₃ domain of a first heavy chain, an amino acid residue is replaced with an amino acid residue having a larger side chain (e.g. tyrosine or tryptophan) creating a projection from the surface (“knob”) and ii) in the CH₃ domain of a second heavy chain, an amino acid residue is replaced with an amino acid residue having a smaller side chain (e.g. alanine or threonine), thereby generating a cavity (“hole”) within at interface in the second CH₃ domain within which the protruding side chain of the first CH₃ domain (“knob”) is received by the cavity in the second CH₃ domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide (e.g. an IL2 ortholog) on a first Fc monomer with a “knob” modification and a second polypeptide on the second Fc monomer possessing a “hole” modification to facilitate the expression of heterodimeric polypeptide conjugates.

The Fc region can be “lytic” or “non-lytic,” but is typically non-lytic. A non-lytic Fc region typically lacks a high affinity Fc receptor binding site and a C1q binding site. The high affinity Fc receptor binding site of murine IgG Fc includes the Leu residue at position 235 of IgG Fc. Thus, the Fc receptor binding site can be inhibited by mutating or deleting Leu 235. For example, substitution of Glu for Leu 235 inhibits the ability of the Fc region to bind the high affinity Fc receptor. The murine C1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fc receptor binding site and a C1q binding site. The high affinity Fc receptor binding site includes the Leu residue at position 235 of IgG Fc, and the C1q binding site includes the Glu 318, Lys 320, and Lys 322 residues of IgG 1. Lytic IgG Fc has wild type residues or conservative amino acid substitutions at these sites. Lytic IgG Fc can target cells for antibody dependent cellular cytotoxicity or complement directed cytolysis (CDC). Appropriate mutations for human IgG are also known (see, e.g., Morrison et al., The Immunologist 2:119-124, 1994; and Brekke et al., The Immunologist 2: 125, 1994).

In certain embodiments, the amino- or carboxyl-terminus of an IL2 ortholog of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

### Targeted IL2 Orthologs:

In some embodiments, the IL2 ortholog is provided as a fusion protein with a polypeptide sequence (“targeting domain”) that selectively binds to a cell surface molecule of a particular cell type or tissue expressing. In some embodiments the IL2 ortholog and targeting domain of the targeted IL2 ortholog fusion protein may optionally incorporate a linker molecule of from 1-40, alternatively 2-20, alternatively 5-20, alternatively 10-20, or alternatively 4-8 amino acids between the IL2 ortholog sequence and the sequence of the targeting domain of the fusion protein.

In other embodiments, a targeted orthogonal IL-2 fusion protein may comprise as antibody or antigen-binding portion thereof wherein the antibody or antigen-binding component of the chimeric protein can serve as a targeting moiety. For example, it can be used to localize the chimeric protein to a particular subset of cells or target molecule. Methods of generating cytokine-antibody chimeric polypeptides are described, for example, in U.S. Pat. No. 6,617,135.

In some embodiments, the targeting domain of the IL2 ortholog fusion protein specifically binds to a cell surface molecule of a tumor cell. In one embodiment wherein the ECD of the CAR of a CAR-T cell specifically binds to CD-19, the IL2 ortholog may be provided as a fusion protein with a CD-19 targeting moiety. For example, in one embodiment wherein the ECD of the CAR of an CAR-T cell is an scFv molecule that provides specific binding to CD-19, the IL2 ortholog is provided as a fusion protein with a CD-19 targeting moiety such as a single chain antibody (e.g., an scFv or VHH) that specifically binds to CD-19.

In some embodiments, the fusion protein comprises an IL-2 mutein and the anti-CD19 scFv FMC63 (Nicholson, et al. (1997) Mol Immunol 34: 1157-1165). Similarly, in some embodiments wherein the ECD of the CAR of an CAR-T cell specifically binds to BCMA, the IL2 ortholog is provided as a fusion protein with a BCMA targeting moiety, such as antibody comprising the CDRs of anti-BMCA antibodies as described in in Kalled, et al. (U.S. Pat. No. 9,034,324 issued May 9, 2015) or antibodies comprising the CDRs as described in Brogdon, et al., (U.S. Pat. No. 10,174,095 issued Jan. 8, 2019). In some embodiments the IL2 ortholog is provided as a fusion protein with a GD2 targeting moiety, such as an antibody comprising the CDRs of described in Cheung, et al., (U.S. Pat. No. 9,315,585 issued Apr. 19, 2016) or the CDRs derived from ME36.1 (Thurin et al., (1987) Cancer Research 47:1229-1233), 14G2a, 3F8 (Cheung, et al., 1985 Cancer Research 45:2642-2649), hu14.18, 8B6, 2E12, or ic9.

In an alternative embodiment, the targeted IL2 orthologs of the present disclosure may be administered in combination with CAR-T cell therapy to provide targeted delivery of the IL2 ortholog to the CAR-T cell based on an extracellular receptor of the CAR-T cell such as by and anti-FMC63 antibody to target the IL2 activity to the CAR-T cells and rejuvenate exhausted CAR-T cells in vivo. Consequently, embodiments of the present disclosure include targeted delivery of IL2 orthologs by conjugation of such IL2 orthologs to antibodies or ligands that are designed to interact with specific cell surface molecules of CAR-T cells. An example of such a molecule would an anti-FMC63-hIL2 ortholog.

In other embodiments, the chimeric polypeptide includes the mutant IL-2 polypeptide and a heterologous polypeptide that functions to enhance expression or direct cellular localization of the mutant IL-2 polypeptide, such as the Aga2p agglutinin subunit (see, e.g., Boder and Wittrup, Nature Biotechnol. 15:553-7, 1997).

In some embodiments, the IL2 ortholog is provided as a fusion protein with a polypeptide sequence (“targeting domain”) to facilitate selective binding to particular cell type or tissue expressing a cell surface molecule that specifically binds to such targeting domain, optionally incorporating a linker molecule of from 1-40 amino acids between the IL2 ortholog sequence and the sequence of the targeting domain of the fusion protein. In one embodiment, the targeting domain of the IL2 ortholog fusion protein specifically binds to a cell surface molecule of the cell type that is targeted by the CAR-T cell expressing the orthogonal CD122. For example, in the event that the orthogonal CD122 CAR-T cell comprises a CAR with an ECD that specifically bind to CD-19, the targeting domain of the IL2 ortholog fusion protein may also bind to CD-19. Examples of targeting domains would include ligands for cell surface receptors or specific binding molecules antibodies. In one embodiment, the IL2 ortholog fusion protein comprises a molecule that specifically binds to the same cell type for which the engineered cell expressing the orthogonal ligand (e.g., and hoCD122 CAR-T cell) is targeted. In one embodiment wherein the ECD of the CAR of an hoCD122 CAR-T cell specifically binds to CD-19, the IL2 ortholog may be provided as a fusion protein with a CD-19 targeting moiety. For example, in one embodiment wherein the ECD of the CAR of an hoCD122 CAR-T cell is an scFv molecule that provides specific binding to CD-19, the IL2 ortholog is provided as a fusion protein with a CD-19 targeting moiety such as a single chain antibody (e.g., an scFv or VHH) that specifically binds to CD-19. In one embodiment, the fusion protein comprises an IL-10 ortholog and the anti-CD19 sdFv FMC63 (Nicholson, et al. (1997) Mol Immunol 34: 1157-1165). Similarly, in some embodiments wherein the ECD of the CAR of an hoCD122 CAR-T cell specifically binds to BCMA, the IL2 ortholog is provided as a fusion protein with a BCMA targeting moiety, such as antibody comprising the CDRs of anti-BMCA antibodies as described in in Kalled, et al (U.S. Pat. No. 9,034,324 issued May 9, 2015) or antibodies comprising the CDRs as described in Brogdon, et al (U.S. Pat. No. 10,174,095 issued Jan. 8, 2019). In some embodiments, wherein the ECD of the CAR of an hoCD122 CAR-T cell specifically binds to GD2, the IL2 ortholog is provided as a fusion protein with a GD2 targeting moiety, such as an antibody comprising the CDRs of described in Cheung, et al (U.S. Pat. No. 9,315,585 issued Apr. 19, 2016) or the CDRs derived from ME36.1 (Thurin et al (1987) Cancer Research 47:1229-1233), 14G2a, 3F8 (Cheung, et al 1985 Cancer Research 45:2642-2649), hu14.18, 8B6, 2E12, or ic9. In some embodiments, the targeting moiety of the IL2 ortholog fusion protein is the same as that provided by the hoCD122 CAR T cell or it may be different, in particular it may be directed to an alternative antigen expressed on the tumor cell type targeted by the CAR. For example, in the context of a hoCD122 scfv 14G2a GD2 targeted CAR-T cell, the IL2 ortholog may be provided in a targeted fusion construct comprising specific binding domain of another GD2 tumor antigen.

### Specific Expansion of Engineered Cell Populations

Once a lymphocyte (e.g., T-cell) or myeloid cell population has been treated to introduce a polynucleotide encoding the orthogonal CD122 into the endogenous CD122 gene, the resulting modified cells can be specifically expanded by contact with the orthogonal IL-2 ligand such as described above or elsewhere herein. In one embodiment, the present disclosure provides a method of selectively expanding a population of engineered cells (e.g., lymphocytes or myeloid cells) expressing an orthogonal CD122 receptor from a mixed cell population, the method comprising contacting the mixed cell population with an IL2 ortholog of the present disclosure under conditions that facilitate the proliferation of the engineered cell. In one embodiment when the lymphocyte or myeloid cell also expresses CAR-T cell, the orthogonal receptor-expressing CAR lymphocytes (e.g., T cells) or myeloid cells may also be selectively expanded from the background or mixed population of transduced and non-transduced cells through the use of the IL2 orthologs described herein. Expansion of the lymphocytes (e.g., T cells) or myeloid cells for therapeutic applications typically involves culturing the cells in contact with a surface providing an agent that stimulates a CD3 TCR complex associated signal and an agent that stimulates a co-stimulatory molecule on the surface of the T-cell. In conventional practice, engineered T-cells are stimulated prior to administration of the cell therapy product by contacting with CD3/D28, particularly in the preparation of CAR-T cells for use in clinical applications. A wide variety or commercially available products are available to facilitate bead-based activation of T-cells including but not limited to the Invitrogen® CTS Dynabeads® CD3/28 (Life Technologies, Inc. Carlsbad CA) or Miltenyi MACS® GMP ExpAct Treg beads or Miltenyi MACS GMP TransAct™ CD3/28 beads (Miltenyi Biotec, Inc.). Conditions appropriate for T-cell culture are well known in the art. Lin, et al. (2009) Cytotherapy 11(7):912-922; Smith, et al. (2015) Clinical & Translational Immunology 4:e31 published online 16 Jan. 2015. The target cells are maintained under conditions necessary to support growth, for example, an appropriate temperature (e.g., 37° C.) and atmosphere (e.g., air plus 5% CO₂). In some embodiments, the mixed cell population containing engineered T cells expressing the CD122 orthogonal receptor is cultured in the presence of a concentration of the IL2 ortholog for at least 2 hours, alternatively at least 3 hours, alternatively at least 4 hours, alternatively at least 6 hours, alternatively at least 8 hours, alternatively at least 12 hours, alternatively at least 24 hours, alternatively at least 48 hours, alternatively at least 72 hours, or more. The concentration of the IL2 ortholog in ex vivo situations is sufficient to induce cellular proliferation in the cell population. T cell proliferation can be readily assessed by microscopic methods and the determination of the optimal concentration of the IL2 ortholog will depend upon the relative activity of the IL2 ortholog for the orthogonal CD122 receptor.

Where the cells are contacted with the IL2 ortholog in vitro, the cytokine can be added to the engineered cells in a dose and for a period of time sufficient to activate signaling from the receptor, which may utilize the native cellular machinery, e.g. accessory proteins, co-receptors, and the like. Any suitable culture medium may be used. The cells thus activated may be used for any desired purpose, including experimental purposes relating to determination of antigen specificity, cytokine profiling, and the like, and for delivery in vivo.

Where the contacting is performed in vivo, an effective dose of engineered cells, including without limitation CAR-T cells that also express an orthogonal CD122 receptor, can be infused to the recipient, in combination with the administration of the orthogonal cytokine, e.g. IL2 and allowed to contact T cells in their native environment, e.g. in lymph nodes, etc. Dosage and frequency may vary depending on the agent; mode of administration; nature of the IL2 ortholog, and the like. It will be understood by one of skill in the art that such guidelines will be adjusted for the individual circumstances. The dosage may also be varied for route of administration, e.g. intramuscular, intraperitoneal, intradermal, subcutaneous, intravenous infusion and the like. Generally at least about 10⁴ engineered cells/kg are administered, at least about 10⁵ engineered cells/kg; at least about 10⁶ engineered cells/kg, at least about 10⁷ engineered cells/kg, or more.

For the engineered T cells, an enhanced immune response may be manifest as an increase in the cytolytic response of T cells towards the target cells present in the recipient, e.g. towards elimination of tumor cells, infected cells; decrease in symptoms of autoimmune disease; and the like. In some embodiments when the engineered T cell population is to be administered to a subject, the subject is provided with immunosuppressive course of therapy prior to or in combination with the administration of the engineered T cell population. Examples of such immunosuppressive regimens include but are not limited to systemic corticosteroids (e.g., methylprednisolone). Therapies for B cell depletion include intravenous immunoglobulin (IVIG) by established clinical dosing guidelines to restore normal levels of serum immunoglobulin levels. In some embodiments, prior to administration of the CAR-T cell therapy of the present invention, the subject may optionally be subjected to a lymphodepleting regimen. One example of such a lymphodepleting regimen consists of the administration to the subject of fludarabine (30 mg/m² intravenous daily for 4 days) and cyclophosphamide (500 mg/m² IV daily for 2 days starting with the first dose of fludarabine).

Engineered T cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow infusion), intraarterial, intraperitoneal, intrathecal or subcutaneous administration. In the typical practice, the engineered T cells are infused to the subject in a physiologically acceptable medium, normally intravascularly, although they may also be introduced into any other convenient site, where the cells may find an appropriate site for growth. Usually, at least 1×10⁵ cells/kg will be administered, at least 1×10⁶ cells/kg, at least 1×10⁷ cells/kg, at least 1×10⁸ cells/kg, at least 1×10⁹ cells/kg, or more, usually being limited by the number of T cells that are obtained during collection.

For example, exemplary ranges for the administration of T-cells cells for use in the practice of the present invention can range from about 1×10⁵ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10⁷ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

The compositions and methods of the present disclosure also provide a method for the treatment of a subject with a T cell therapy (especially CAR T cell therapy), optionally in the absence of prior lymphodepletion. Lymphodepletion is typically performed in a subject in conjunction with CAR T cell therapy because the subsequent administration of the mixed cell population and the administration of non-specific agents (e.g. IL2) to expand the engineered cell population in the subject in combination with the administration of the cell therapy product acts results in significant systemic toxicity (including cytokine release syndrome or “cytokine storm”) arising from the widespread proliferation and activation of immune cells by administration of agents that result in widespread activation as well as the presence of a substantial fraction of non-engineered cells in the cell therapy product itself. The methods and compositions of the present disclosure obviate this significant hurdle by both (or either) providing a substantially purified population of engineered cells largely devoid of contamination by non-engineered cells when the foregoing ex vivo method is employed and/or the selective activation and expansion of the engineered T cells with the IL2 orthologs which provide substantially reduced off-target effects of non-specific proliferative agents such as IL2.

For example, in the current clinical practice of CAR-T cell therapy, CAR-T cells are commonly administered in combination with lymphodepletion (e.g. by administration of Alemtuzumab (monoclonal anti-CD52), purine analogs, and the like) to facilitate expansion of the CAR-T cells to prior to host immune recovery. In some embodiments, the CAR-T cells may be modified for resistance to Alemtuzumab. In one aspect, the lymphodepletion currently employed in association with CAR-T therapy may be obviated or reduced by the orthogonal ligand expressing CAR-Ts. As noted above, the lymphodepletion is commonly employed to enable expansion of the CAR-T cells. However, the lymphodepletion is also associated with major side effects of CAR-T cell therapy. Because the orthogonal ligand provides a means to selectively expand a particular T-cell population, the need for lymphodepletion prior to administration of the orthogonal ligand expressing CAR-Ts may be reduced. CAR-T cell therapy without or with reduced lymphodepletion prior to administration of the orthogonal ligand expressing CAR-Ts can be employed.

### Methods of Treatment:

The present disclosure further provides a method of preventing or treating a mammalian subject suffering from a disease, disorder or condition by administering to said subject a therapeutically effective amount of hoCD122^{pos}/wt hCD122^{neg} cells in combination with an orthogonal ligand (hoIL2). The administration of the orthogonal ligand to the subject in combination with a population of hoCD122^{pos}/wt hCD122^{neg} cells provides for selective activation and/or proliferation of the hoCD122^{pos}/wt hCD122^{neg} cells in the subject.

In one embodiment, the present disclosure provides a method of treating a subject suffering from a disease, disorder or condition amendable to treatment with CAR-T cell therapy (e.g. cancer) by the administration of orthogonal CD122 expressing lymphocytes (e.g., T-cells) or myeloid cells as described herein in the absence of lymphodepletion prior to administration of the orthogonal ligand CAR-Ts. In one embodiment, the present disclosure provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T-cells from the individual; (b) enriching the biological sample for the presence of T-cells; (c) transfecting the T-cells with one or more expression vectors comprising a nucleic acid sequence encoding a CAR and a nucleic acid sequence encoding an orthogonal CD122 receptor, the antigen targeting domain of the CAR being capable of binding to at least one antigen present on the aberrant population of cells; (d) expanding the population of the orthogonal receptor expressing CAR-T cells ex vivo with an IL2 ortholog; (e) administering a pharmaceutically effective amount of the orthogonal receptor expressing CAR-T cells to the mammal; and (f) modulating the growth of the orthogonal CD122 receptor expressing CAR-T cells by the administration of a therapeutically effective amount of an IL2 ortholog that binds selectively to the orthogonal CD122 receptor expressed on the CAR-T cell. In one embodiment, the foregoing method is associated with lymphodepletion or immunosuppression of the mammal prior to the initiation of the course of CAR-T cell therapy. In another embodiment, the foregoing method is practiced in the absence of lymphodepletion and/or immunosuppression of the mammal.

### Administration of the Orthogonal Ligand:

In embodiments of the therapeutic methods of the present disclosure involve the administration of a pharmaceutical formulation comprising an IL2 ortholog (and/or nucleic acids encoding the IL2 ortholog) to a subject in need of treatment. Administration to the subject may be achieved by intravenous, as a bolus or by continuous infusion over a period of time. Alternative routes of administration include intramuscular, intraperitoneal, intra-cerobrospinal, subcutaneous, intra-articular, intrasynovial, intrathecal, oral, topical, or inhalation routes. The IL2 orthologs also are suitably administered by intratumoral, peritumoral, intralesional, intranodal or perilesional routes or to the lymph, to exert local as well as systemic therapeutic effects.

In some embodiments, subject IL2 orthologs (and/or nucleic acids encoding the IL2 ortholog) can be incorporated into compositions, including pharmaceutical compositions. Such compositions typically include the polypeptide or nucleic acid molecule and a pharmaceutically acceptable carrier. A pharmaceutical composition is formulated to be compatible with its intended route of administration and is compatible with the therapeutic use for which the IL2 ortholog is to be administered to the subject in need of treatment or prophyaxis.

### Formulations of IL2 Orthologs

The IL2 orthologs (or nucleic acids encoding same) of the present disclsoure may be administered to a subject in a pharmaceutically acceptable dosage form. The preferred formulation depends on the intended mode of administration and therapeutic application.

Parenteral Formulations:

In some embodiments, the methods of the present disclosure involve the parental administration of a IL2 ortholog. Examples of parenteral routes of administration include, for example, intravenous, intradermal, subcutaneous, transdermal (topical), transmucosal, and rectal administration. Parenteral formulations comprise solutions or suspensions used for parenteral application can include vehicles the carriers and buffers. Pharmaceutical formulations for parenteral administration include sterile aqueous solutions (where water soluble) or dispersions and sterile powders for the extemporaneous preparation of sterile injectable solutions or dispersion. The parenteral preparation can be enclosed in ampoules, disposable syringes or multiple dose vials made of glass or plastic. In one embodiment, the formulation is provided in a prefilled syringe for parenteral administration.

Oral Formulations:

Oral compositions, if used, generally include an inert diluent or an edible carrier.

For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel™, or corn starch; a lubricant such as magnesium stearate or Sterotes™; a glidant such as colloidal silicon dioxide; a sweetening agent such as sucrose or saccharin; or a flavoring agent such as peppermint, methyl salicylate, or orange flavoring.

Inhalation Formulations:

In the event of administration by inhalation, subject IL2 orthologs, or the nucleic acids encoding them, are delivered in the form of an aerosol spray from pressured container or dispenser which contains a suitable propellant, e.g., a gas such as carbon dioxide, or a nebulizer. Such methods include those described in U.S. Pat. No. 6,468,798.

Mucosal and Transdermal:

Systemic administration of the subject IL2 orthologs or nucleic acids can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories suppositories (e.g., with conventional suppository bases such as cocoa butter and other glycerides) or retention enemas for rectal delivery. For transdermal administration, the active compounds are formulated into ointments, salves, gels, or creams as generally known in the art and may incorporate permeation enhancers such as ethanol or lanolin.

Extended Release and Depot Formulations:

In some embodiments of the method of the present disclosure, the IL2 ortholog is administered to a subject in need of treatment in a formulation to provide extended release of the IL2 ortholog agent. Examples of extended release formulations of the injectable compositions can be brought about by including in the composition an agent which delays absorption, for example, aluminum monostearate and gelatin. In one embodiment, the subject IL2 orthologs or nucleic acids are prepared with carriers that will protect the mutant IL-2 polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, the IL2 ortholog formulation is provided in accordance with the teaching of Fernandes and Taforo, U.S. Pat. No. 4,604,377 issued Aug. 5, 1986 the teaching of which is herein incorporated by reference. And Yasui, et al., U.S. Pat. No. 4,645,830.

### Administration of Nucleic Acids Encoding the Ortholog:

Alternative to the administration to a subject of a IL2 ortholog protein pharmaceutical formulation comprising an IL2 ortholog, the IL2 ortholog may be provided to a subject by the administration of pharmaceutically acceptable formulation of a nucleic acid construct encoding the IL2 ortholog to the subject to achieve continuous exposure of the subject to the selective IL2 ortholog. The administration of a recombinant vector encoding the IL2 ortholog provides for extended delivery of the IL2 ortholog to the subject and prolonged activation of the corresponding cells engineered to express the cognate orthogonal receptor associated with such IL2 ortholog. In some embodiments of the method of the present disclosure, nucleic acids encoding the IL2 ortholog is administered to the subject by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996

Non-Viral Vectors Encoding the Ortholog: In one embodiment, the IL2 ortholog may be administered to a subject in the form of nucleic acid expression construct for the IL2 ortholog in a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Critical Reviews of Therapeutic Drug Carrier Systems 14:173-206); polymer coated liposomes (Marin et al., U U.S. Pat. No. 5,213,804, issued May 25, 1993; Woodle, et al., U U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U U.S. Pat. No. 5,279,833, issued Jan. 18, 1994; Gebeyehu et al., U.S. Pat. No. 5,334,761, issued Aug. 2, 1994). In one embodiment, the nucleic acid sequence in the non-viral vector system encoding the IL2 receptor is under control of a regulatable promoter, inducible promoter, tissue specific or tumor specific promoter, or temporally regulated promoter.

Viral Vectors Encoding the Ortholog:

In another embodiment, IL2 ortholog may be administered to a subject in the form of nucleic acid expression construct in viral vector encoding the IL2 ortholog. The terms “viral vector” and “virus” are used interchangeably herein to refer to any of the obligate intracellular parasites having no protein-synthesizing or energy-generating mechanism. The viral genome may be RNA or DNA contained with a coated structure of protein of a lipid membrane. The terms virus(es) and viral vector(s) are used interchangeably herein. The viruses useful in the practice of the present invention include recombinantly modified enveloped or nonenveloped DNA and RNA viruses, preferably selected from baculoviridiae, parvoviridiae, picornoviridiae, herpesviridiae, poxviridae, or adenoviridiae. The viruses are modified by recombinant DNA techniques to include expression of exogenous transgenes (e.g. a nucleic acid sequence encoding the IL2 ortholog) and may be engineered to be replication deficient, conditionally replicating or replication competent. Minimal vector systems in which the viral backbone contains only the sequences need for packaging of the viral vector and may optionally include a transgene expression cassette may also be employed. The term “replication deficient” refers to vectors that are highly attenuated for replication in a wild type mammalian cell. In order to produce such vectors in quantity, a producer cell line is generally created by co-transfection with a helper virus or genomically modified to complement the missing functions. The term “replication, competent viral vectors” refers to a viral vector that is capable of infection, DNA replication, packaging and lysis of an infected cell. The term “conditionally replicating viral vectors” is used herein to refer to replication competent vectors that are designed to achieve selective expression in particular cell types. Such conditional replication may be achieved by operably linking tissue specific, tumor specific or cell type specific or other selectively induced regulatory control sequences to early genes (e.g., the E1 gene of adenoviral vectors). Infection of the subject with the recombinant virus or non-viral vector can provide for long term expression of the IL2 ortholog in the subject and provide continuous selective maintenance of the engineered T cells expressing the CD122 orthogonal receptor. In one embodiment, the nucleic acid sequence in the viral vector system encoding the IL2 receptor is under control of a regulatable promoter, inducible promoter, tissue specific or tumor specific promoter, or temporally regulated promoter.

### CAR-T Cells

In some embodiments, the human immune cell expressing the orthogonal receptor is a T-cell (e.g., human T-cell) which has also been modified to surface express a chimeric antigen receptor (a ‘CAR-T’ cell). In some embodiments, the T-cell is modified to express the orthogonal CD122 and to express the CAR-T in the same procedure. For example, in some embodiments, a polynucleotide encoding the orthogonal CD122 and a polynucleotide encoding the CAR-T are introduced into a T-cell and the resulting cell product can be selectively expanded as described herein, using an IL-2 ortholog, a CAR-T ligand, or both. In some embodiments, a polynucleotide encoding the orthogonal CD122 and the CAR-T, e.g., separated by appropriate expression control elements, is introduced into a T-cell such that the T-cell recipient express the orthogonal CD122 (and no longer expresses the endogenous CD122) and the CAR protein.

CAR Signal Sequence

As noted above, the CAR may comprise a signal peptide. In the practice of the present invention any eukaryotic signal peptide sequence may be employed. The signal peptide may be derived from native signal peptides of surface expressed proteins. In one embodiment of the invention, the signal peptide of the CAR is the signal peptide selected from the group consisting of human serum albumin signal peptide, prolactin albumin signal peptide, the human IL2 signal peptide, human trypsinogen-2, human CD-5, the human immunoglobulin kappa light chain, human azurocidin, Gaussia luciferase and functional derivatives thereof. Particular amino acid substitutions to increase secretion efficiency using signal peptides are described in Stern, et al. (2007) Trends in Cell and Molecular Biology 2:1-17 and Kober, et al. (2013) Biotechnol Bioeng. 1110(4):1164-73. Alternatively, the signal peptide may be a synthetic sequence prepared in accordance established principles. See e.g., Nielsen, et al. (1997) Protein Engineering 10(1):1-6 (Identification of prokaryotic and eukaryotic signal peptides and prediction of their cleavage sites); Bendtsen, et al (2004) J. Mol. Biol 340(4):783-795 (Improved Prediction of Signal Peptides SignalP 3.0); Petersen, et al (2011) Nature Methods 8:785-796 (Signal P 4.0; discriminating signal peptides from transmembrane regions).

CAR Antigen Binding Domain (ABD)

As used herein, the term antigen binding domain (ABD) refers to a polypeptide that contains at least one binding domain that specifically binds to at least one antigen expressed on the surface of a target cell. In some embodiments, the ABD comprises a polypeptide with two binding domains that selectively bind to the same antigen or two different antigens on the surface of the target cells. The ABD may be any polypeptide that specifically binds to one or more antigens expressed on the surface of a target cell. The ABD of the CAR may be monovalent or multivalent and comprise one or multiple (e.g. 1, 2, or 3) polypeptide sequence (e.g. scFv, VHH, ligand) that specifically bind to a cell surface tumor antigen. In some embodiments, tumor antigens and CARs comprising ABDs that selectively hind to such cell surface tumor are known in the art (see, e.g., Dotti, et al., Immunol Rev, 2014 January, 257(1) The methods and compositions of the present disclosure are useful in conjunction with CAR therapy wherein the ABD of the CAR specifically binds a tumor antigen including but not limited to CD123, CD19, CD20, BCMA, CD22, CD30, CD70, Lewis Y, GD3, GD3, mesothelin, ROR CD44, CD171, EGP2, EphA2, ErbB2, ErbB3/4, FAP, FAR IL11Ra, PSCA, PSMA, NCAM, HER2, NY-ESO-1, MUC1, CD123, FLT3, B7-H3, CD33, IL1RAP, CLL1 (CLEC12A)PSA, CEA, VEGF, VEGF-R2, CD22, ROR1, GPC3, mesothelin, c-Met, Glycolipid F77, FAP, EGFRvIII, MAGE A3, 5T4, WT1, KG2D ligand, a folate receptor (FRa), and Wnt1 antigens. Antibodies reactive with these targets are well known in the literature and one of skill in the art is capable of isolating the CDRs from such antibodies for the construction of polypeptide sequences of single chain antibodies (e.g. scFvs, CDR grafted VHHs and the like) that may be incorporated into the ABD of the CAR.

In one embodiment, the ABD is a single chain Fv (ScFv). An ScFv is a polypeptide comprised of the variable regions of the immunoglobulin heavy and light chain of an antibody covalently connected by a peptide linker (Bird, et al. (1988) Science 242:423-426; Huston, et al. (1988) PNAS(USA) 85:5879-5883; S-z Hu, et al. (1996) Cancer Research, 56, 3055-3061; Ladner, U.S. Pat. No. 4,946,778 issued Aug. 7, 1990). The preparation of an anti-targeting antigen ScFv involves the identification of a monoclonal antibody against the targeting antigen for from which the anti-targeting antigen ScFv is derived. The generation of monoclonal antibodies and isolation of hybridomas is a technique well known to those of skill in the art. See e.g. Monoclonal Antibodies: A Laboratory Manual, Second Edition, Chapter 7 (E. Greenfield, Ed. 2014 Cold Spring Harbor Press). Immune response may be enhanced through co-administration of adjuvants well known in the art such as alum, aluminum salts, or Freund's, SP-21, etc. Antibodies generated may be optimized to select for antibodies possessing particular desirable characteristics through techniques well known in the art such as phage display and directed evolution. See, e.g. Barbas, et al. (1991) PNAS(USA) 88:7978-82; Ladner, et al. U.S. Pat. No. 5,223,409 issued Jun. 29, 1993; Stemmer, W. (1994) Nature 370:389-91; Garrard U.S. Pat. No. 5,821,047 issued Oct. 13, 1998; Camps, et al. (2003) PNAS(USA) 100(17): 9727-32; Dulbecco U.S. Pat. No. 4,593,002 issued Jun. 3, 1986; McCafferty U.S. Pat. No. 6,806,079 issued Oct. 19, 2004; McCafferty, U.S. Pat. No. 7,635,666 issued Dec. 22, 2009; McCafferty, U.S. Pat. No. 7,662,557 issued Feb. 16, 2010; McCafferty, U.S. Pat. No. 7,723,271 issued May 25, 2010; and/or McCafferty U.S. Pat. No. 7,732,377. The generation of ScFvs based on monoclonal antibody sequences is well known in the art. See, e.g. The Protein Protocols Handbook, John M. Walker, Ed. (2002) Humana Press Section 150 “Bacterial Expression, Purification and Characterization of Single-Chain Antibodies” Kipriyanov, S.

In another embodiment, the ABD is a single domain antibody obtained through immunization of a camel or llama with a targeting antigen. Muyldermans, S. (2001) Reviews in Molecular Biotechnology 74: 277-302.

Alternatively, the ABD may be generated wholly synthetically through the generation of peptide libraries and isolating compounds having the desired target cell antigen binding properties. Such techniques are well known in the scientific literature. See, e.g. Wigler, et al. U.S. Pat. No. 6,303,313 B1 issued Nov. 12, 1999; Knappik, et al., U.S. Pat. No. 6,696,248 B1 issued Feb. 24, 2004, Binz, et al (2005) Nature Biotechnology 23:1257-1268; Bradbury, et al. (2011) Nature Biotechnology 29:245-254.

In addition to the ABD having affinity for the target cell expressed antigen, the ARD may also have affinity for additional molecules. For example, an ARD of the present invention may be bi-specific, i.e. have capable of providing for specific binding to a first target cell expressed antigen and a second target cell expressed antigen. Examples of bivalent single chain polypeptides are known in the art. See, e.g. Thirion, et al. (1996) European J. of Cancer Prevention 5(6):507-511; DeKruif and Logenberg (1996) J. Biol. Chem 271(13)7630-7634; and Kay, et al. United States Patent Application Publication Number 2015/0315566 published Nov. 5, 2015.

The ABD may have affinity for more than one target antigen. For example, an ABD of the present invention may comprise chimeric bispecific binding members, i.e. have capable of providing for specific binding to a first target cell expressed antigen and a second target cell expressed antigen. Non-limiting examples of chimeric bispecific binding members include bispecific antibodies, bispecific conjugated monoclonal antibodies (mab)₂, bispecific antibody fragments (e.g., F(ab)₂, bispecific scFv, bispecific diabodies, single chain bispecific diabodies, etc.), bispecific T cell engagers (BiTE), bispecific conjugated single domain antibodies, micabodies and mutants thereof, and the like. Non-limiting examples of chimeric bispecific binding members also include those chimeric bispecific agents described in Kontermann (2012) MAbs. 4(2): 182-197; Stamova et al. (2012) Antibodies, 1(2), 172-198; Farhadfar et al. (2016) Leuk Res. 49:13-21; Benjamin et al. Ther Adv Hematol. (2016) 7(3):142-56; Kiefer et al. Immunol Rev. (2016) 270(1):178-92; Fan et al. (2015) J Hematol Oncol. 8:130; May et al. (2016) Am J Health Syst Pharm. 73(1):e6-e13. In some embodiments, the chimeric bispecific binding member is a bivalent single chain polypeptides. See, e.g. Thirion, et al. (1996) European J. of Cancer Prevention 5(6):507-511; DeKruif and Logenberg (1996) J. Biol. Chem 271(13)7630-7634; and Kay, et al. United States Patent Application Publication Number 2015/0315566 published Nov. 5, 2015.

In some instances, a chimeric bispecific binding member may be a CAR T cell adapter. As used herein, by “CAR T cell adapter” is meant an expressed bispecific polypeptide that binds the antigen recognition domain of a CAR and redirects the CAR to a second antigen. Generally, a CAR T cell adapter will have to binding regions, one specific for an epitope on the CAR to which it is directed and a second epitope directed to a binding partner which, when bound, transduces the binding signal activating the CAR. Useful CAR T cell adapters include but are not limited to e.g., those described in Kim et al. (2015) J Am Chem Soc. 137(8):2832-5; Ma et al. (2016) Proc Natl Acad Sci USA. 113(4):E450-8 and Cao et al. (2016) Angew Chem Int Ed Engl. 55(26):7520-4

In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119. Another antibody is S58 (anti-GD2, neuroblastoma). CotaraTM [Perregrince Pharmaceuticals] is a monoclonal antibody described for treatment of recurrent glioblastoma. In some embodiments the ABD of the CAR comprises the scFvFMC-63 and humanize variants thereof

Linkers/Hinge

CARs useful in the practice of the present invention may optionally include one or more polypeptide spacers linking the domains of the CAR, in particular the linkage between the ARD to the transmembrane spanning domain of the CAR. Although not an essential element of the CAR structure, the inclusion of a spacer domain is generally considered desirable to facilitate antigen recognition by the ARD. Moritz and Groner (1995) Gene Therapy 2(8) 539-546. As used in conjunction with the CAR-T T cell technology described herein, the terms “linker”, “linker domain” and “linker region” refer to an oligo- or polypeptide region from about 1 to 100 amino acids in length, which links together any of the domains/regions of the CAR of the disclosure. Linkers may be composed of flexible residues like glycine and serine so that the adjacent protein domains are free to move relative to one another. Certain embodiments comprise the use of linkers of longer length when it is desirable to ensure that two adjacent domains do not sterically interfere with each another.

In some embodiments, the linkers are non-cleavable, while in others they are cleavable (e.g., 2A linkers (for example T2A)), 2A-like linkers or functional equivalents thereof, and combinations of the foregoing. There is no particular sequence of amino acids that is necessary to achieve the spacer function but the typical properties of the spacer are flexibility to enable freedom of movement of the ARD to facilitate targeting antigen recognition. Similarly, it has been found that there is there is substantial leniency in spacer length while retaining CAR function. Jensen and Riddell (2014) Immunol. Review 257(1) 127-144. Sequences useful as spacers in the construction of CARs useful in the practice of the present invention include but are not limited to the hinge region of IgG1, the immunoglobulin1 CH₂—CH₃ region, IgG4 hinge-CH₂—CH₃, IgG4 hinge-CH₃, and the IgG4 hinge. The hinge and transmembrane domains may be derived from the same molecule such as the hinge and transmembrane domains of CD8-alpha. Imai, et al. (2004) Leukemia 18(4):676-684. Embodiments of the present disclosure are contemplated wherein the linkers include the picornaviral 2A-like linker, CHYSEL sequences of porcine teschovirus (P2A), Thosea asigna virus (T2A), or combinations, variants and functional equivalents thereof. In still further embodiments, the linker sequences comprise Asp-Val/Ile-Glu-X-Asn-Pro-Gly^{(2A)}-pro^{(2B)} motif (SEQ ID NO: 17), which results in cleavage between the 2A glycine and the 2B proline.

CAR Transmembrane Domain

CARs can further comprise a transmembrane domain joining the ABD (or linker, if employed) to the intracellular cytoplasmic domain of the CAR. The transmembrane domain is comprised of any polypeptide sequence which is thermodynamically stable in a eukaryotic cell membrane. The transmembrane spanning domain may be derived from the transmembrane domain of a naturally occurring membrane spanning protein or may be synthetic. In designing synthetic transmembrane domains, amino acids favoring alpha-helical structures are preferred. Transmembrane domains useful in construction of CARs are comprised of approximately 10, 11, 12, 13, 14, 15, 16, 17, 18, 19, 20, 21, 22, 22, 23, or 24 amino acids favoring the formation having an alpha-helical secondary structure. Amino acids having favoring alpha-helical conformations are well known in the art. See, e.g Pace, et al. (1998) Biophysical Journal 75: 422-427. Amino acids that are particularly favored in alpha helical conformations include methionine, alanine, leucine, glutamate, and lysine. In some embodiments, the CAR transmembrane domain may be derived from the transmembrane domain from type I membrane spanning proteins, such as CD3, CD4, CD8, CD28, etc.

CAR Intracellular Signaling Domain

The cytoplasmic domain of the CAR polypeptide comprises one or more intracellular signal domains. In one embodiment, the intracellular signal domains comprise the cytoplasmic sequences of the T-cell receptor (TCR) and co-receptors that initiate signal transduction following antigen receptor engagement and functional derivatives and sub-fragments thereof. A cytoplasmic signaling domain, such as those derived from the T cell receptor zeta-chain, is employed as part of the CAR in order to produce stimulatory signals for T lymphocyte or myeloid cell proliferation and effector function following engagement of the chimeric receptor with the target antigen. Examples of cytoplasmic signaling domains include but are not limited to the cytoplasmic domain of CD27, the cytoplasmic domain S of CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase, the human CD3 ζ-chain, cytoplasmic domain of CD134 (also referred to as OX40 and TNFRSF4), FcεR1γ and β chains, MB1 (Igα) chain, B29 (Igβ) chain, etc.), CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28.

Co-Stimulatory Domain

In some embodiments, the CAR may also provide a co-stimulatory domain. The term “co-stimulatory domain”, refers to a stimulatory domain, typically an endodomain, of a CAR that provides a secondary non-specific activation mechanism through which a primary specific stimulation is propagated. The co-stimulatory domain refers to the portion of the CAR which enhances the proliferation, survival or development of memory cells. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. (2013) Nat Rev Immunol 13(4):227-42. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof.

CARs are often referred to as first, second, third or fourth generation. The term first-generation CAR refers to a CAR wherein the cytoplasmic domain transmits the signal from antigen binding through only a single signaling domain, for example a signaling domain derived from the high-affinity receptor for IgE FcεR1γ or the CD3ζ chain. The domain contains one or three immunoreceptor tyrosine-based activating motif(s) [ITAM(s)] for antigen-dependent T-cell activation. The ITAM-based activating signal endows T-cells with the ability to lyse the target tumor cells and secret cytokines in response to antigen binding. Second-generation CARs include a co-stimulatory signal in addition to the CD3 ζ signal. Coincidental delivery of the delivered co-stimulatory signal enhances cytokine secretion and antitumor activity induced by CAR-transduced T-cells. The co-stimulatory domain is usually be membrane proximal relative to the CD3ζ domain. Third-generation CARs include a tripartite signaling domain, comprising for example a CD28, CD3, OX40 or 4-1BB signaling region. In fourth generation, or “armored car” CAR T-cells are further modified to express or block molecules and/or receptors to enhance immune activity such as the expression of IL-12, IL-18, IL-7, and/or IL-10; 4-1BB ligand, CD-40 ligand.

Examples of intracellular signaling domains comprising may be incorporated into the CAR of the present invention include (amino to carboxy): CD3ζ; CD28-41BB—CD3ζ; CD28-OX40-CD3ζ; CD28-41BB-CD3ζ; 41BB—CD-28-CD3ζ and 41BB-CD3ζ.

Furthermore, in addition to the more conventional first and second generation CARS, the term CAR includes CAR variants including but not limited split CARs, ON-switch CARS, bispecific or tandem CARs, inhibitory CARs (iCARs) and induced pluripotent stem (iPS) CAR-T cells.

The term “Split CARs” refers to CARs wherein the extracellular portion, the ABD and the cytoplasmic signaling domain of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR molecules and derivatives thereof (i.e., CAR variants) are described, e.g., in PCT Application Nos. US2014/016527, US1996/017060, US2013/063083; Fedorov et al. Sci Transl Med (2013) 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The term “bispecific or tandem CARs” refers to CARs which include a secondary CAR binding domain that can either amplify or inhibit the activity of a primary CAR.

The term “inhibitory chimeric antigen receptors” or “iCARs” are used interchangeably herein to refer to a CAR where binding iCARs use the dual antigen targeting to shut down the activation of an active CAR through the engagement of a second suppressive receptor equipped with inhibitory signaling domains of a secondary CAR binding domain results in inhibition of primary CAR activation. T cells with specificity for both tumor and off-target tissues can be restricted to tumor only by using an antigen-specific iCAR introduced into the I cells to protect the off-target tissue (Fedorov; et al., (2013). Science Translational Medicine, 5:215). Inhibitory CARs (iCARs) are designed to regulate CAR-T cells activity through inhibitory receptors signaling modules activation. This approach combines the activity of two CARs, one of which generates dominant negative signals limiting the responses of CAR-T cells activated by the activating receptor. iCARs can switch off the response of the counteracting activator CAR when bound to a specific antigen expressed only by normal tissues. In this way, iCARs-T cells can distinguish cancer cells from healthy ones, and reversibly block functionalities of transduced T cells in an antigen-selective fashion. CTLA-4 or PD-1 intracellular domains in iCARs trigger inhibitory signals on T lymphocytes or myeloid cells, leading to less cytokine production, less efficient target cell lysis, and altered lymphocyte or myeloid cell motility. In some embodiments, the iCAR comprises an single chain antibody (e.g. scFv, VHH, etc) that specifically binds to an inhibitory antigen, one or more intracellular derived from the ICDs Immunoinhibitory receptors (including but not limited to CTLA-4 PD-1, LAG-3, 2B4 (CD244), BMA (CD272). TIM-3; TGFbeta receptor dominant negative analog etc.) via a transmembrane region that inhibits cell function specifically upon antigen recognition.

The term “tandem CAR” or “TanCAR” refers to CARs which mediate bispecific activation of T cells through the engagement of two chimeric receptors designed to deliver stimulatory or costimulatory signals in response to an independent engagement of two different tumor associated antigens.

Typically, the chimeric antigen receptor T-cells (CAR-T cells) are T-cells which have been recombinantly modified by transduction with an expression vector encoding a CAR in substantial accordance with the teaching above.

In some embodiments, an engineered T cell is allogeneic with respect to the individual that is treated. See, e.g., Graham et al. (2018) Cell 7(10) E155. In some embodiments an allogeneic engineered T cell is partially or fully HLA matched. However not all patients have a fully matched donor and a cellular product suitable for all patients independent of HLA type provides an alternative.

Because the cell product may consist of a subject's own T-cells, the population of the cells to be administered is to the subject is necessarily variable. Additionally, since the CAR-T cell agent is variable, the response to such agents can vary and thus involves the ongoing monitoring and management of therapy related toxicities which are managed with a course of pharmacologic immunosuppression or B cell depletion prior to the administration of the CAR-T cell treatment. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection. The engineered cells may be infused to the subject in any physiologically acceptable medium by any convenient route of administration, normally intravascularly, although they may also be introduced by other routes, where the cells may find an appropriate site for growth

If the T cells used as described herein are allogeneic T cells, such cells may be modified to reduce graft versus host disease. For example, the engineered cells may be TCRαβ receptor knock-outs achieved by gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-CreI homing endonuclease, etc. See, for example, Eyquem et al. (2017) Nature 543:113-117, in which the TRAC coding sequence is replaced by a CAR coding sequence; and Georgiadis et al. (2018) Mol. Ther. 26:1215-1227, which linked CAR expression with TRAC disruption by clustered regularly interspaced short palindromic repeats (CRISPR)/Cas9 without directly incorporating the CAR into the TRAC loci. See, also, Stadtmauer et al. Science Vol. 367, Issue 6481 (2020) An alternative strategy to prevent GVHD modifies T cells to express an inhibitor of TCRαβ signaling, for example using a truncated form of CD3ζ as a TCR inhibitory molecule. In some embodiments, the lymphotyes described herein are deleted for one or more of T cell receptor alpha (TCRA), T cell receptor beta (TCRB), PD-1, cytotoxic T-lymphocyte-associated protein 4 (CTLA4), beta2 microglobulin (B2M), LAG3, TIM3, TGFBR2, FAS, TET2, SOCS1, TCEB2, RASA2, CBLB, ADORA2A, PTPN2, KDR, or FAM105A. Examples of the deletion of CLTA4, B2M, PD-1, TCRA and TCRB can be found, for example, in US Patent Publication No. 2016/0348073.

### Methods of Treatment

### Selective Activation Of hoCD122+/nCD122− Orthogonal Cells

In some embodiments, the disclosure provides a method to selectively activate and/or stimulate the proliferation of an engineered human immune cell comprising a genomically-integrated polynucleotide encoding an orthogonal human CD122 (hoCD122) polypeptide by contacting the in a mixed population of cells by contacting the mixed population of cells with an IL2 ortholog that is a cognate ligand for the orthogonal CD122 of the orthogonal cell. In some embodiments, the present disclosure provides an engineered human immune cell comprising a genomically-integrated polynucleotide encoding an hoCD122 operably linked to at least one expression control sequence functional in the human immune cell to effect expression of hoCD122 in the engineered human immune cell.

Treatment of Disease Disorder of Condition with Orthogonal Cells

In some embodiments, the present disclosure provides therapeutic methods to the treatment of a subject suffering from a disease, disorder or condition, the method comprising the administration to said subject a population engineered human immune cell comprising a genomically-integrated polynucleotide encoding an orthogonal human CD122 (hoCD122) polypeptide in combination with the administration of an IL2 ortholog that is a cognate ligand for the orthogonal CD122 expressed on said orthogonal cells.

In some embodiments, the present disclosure provides therapeutic methods for the treatment of a subject suffering from a neoplastic disease, disorder or condition, the method comprising the the method comprising the administration to said subject a population engineered human immune cell comprising a genomically-integrated polynucleotide encoding an orthogonal human CD122 (hoCD122) polypeptide, wherein the engineered cells are human orthogonal TILs (hoTILs), in combination with a therapeutically effective amount of a human IL2 ortholog that is a cognate ligand for the orthogonal human CD122 expressed on said hoTILs.

In some embodiments, the present disclosure provides therapeutic methods for the treatment of a subject suffering from a neoplastic disease, disorder or condition, the method comprising the method comprising the administration to said subject a population engineered human immune cell comprising a genomically-integrated polynucleotide encoding an orthogonal human CD122 (hoCD122) polypeptide, wherein the engineered cells are human orthogonal CAR-T (hoCAR-T) cells in combination with a therapeutically effective amount of a human IL2 ortholog that is a cognate ligand for the orthogonal human CD122 expressed on said hoTILs. In some embodiments, the hoCAR-T cells of the method is selected from the group consisting of CD19 hoCAR-T cells, CD20, hoCAR-T cells, BCMA hoCAR-T cells, or GPC3 hoCAR-T cells.

In some cases, the subject is suffering from a neoplastic disease and the orthogonal human immune cells are CD8+ T cells. In some cases, the subject is suffering from an autoimmune disease the orthogonal human immune cells are Treg cells. In some cases, the engineered immune cells are hoCAR-T cells. In another aspect, the invention feature cytotoxic cell, e.g., a naturally or non-naturally occurring T cell, NK cell or cytotoxic T cell or cell of an NK cell line, e.g., NK92, comprising (a) a first KIR-CAR described herein. In one embodiment, said cytotoxic cell is cell. In one embodiment, said cytotoxic cell is an NK cell. In one embodiment, said cytotoxic cell is from an NK cell line, e.g., an NK92 cell.

### Treatment Optionally In Absence of Lymphodepletion:

In some embodiments, the methods of the present disclosure optionally further comprise the step of lymphodepletion prior to the administration of the engineered orthogonal cells to the subject. Lymphodepletion is typically performed in a subject in conjunction with adoptive cell therapy by the administration of a mixed cell population comprising the CAR-Ts or TILs in combination with the administration of non-specific agents (e.g. IL2) to support the CAR-Ts or TILs. Studies suggest that lymphodepletion may have therapeutic benefits in the context of adoptive cell transfer. It is reported that lymphodepletion depletes Tregs, removes cellular “sinks”, provided physical space for the adoptively transferred cells to proliferate in the subject, reduces the competition for homeostatic cytokines such as IL-7 and IL-15 and reduces immunosuppressive lymphoid and myeloid populations. However, it should be noted that lymphodepletion is associated with certain serious toxicities associated with adoptive cell transfer treatment. Lymphodepleting regimens cause a short, but deep lymphopenia and neutropenia, with full bone marrow recovery within 7-10 days, typically not requiring hematopoietic stem cell support. In those circumstance where lymphodepletion is deemed necessary by the healthcare professional, the subject should be closely monitored to address any resulting toxicities.

In those circumstances where lymphodepletion is employed in the context of the therapeutic method, lymphodepletion may be achieved by treating said subject with a lymphodepleting treatment regimen comprising anti-CD52 antibodies, purine analogs, and the like. In some embodiments, the lymphodepleting treatment regimen is a lymphodepleting non-myeloablative chemotherapeutic regimen (NMA chemotherapy). One example of a lymphodepleting non-myeloablative chemotherapeutic regimen (NMA chemotherapy) commonly used in clinical practice comprises the following steps: approximately of 2 days intravenous administration of cyclophosphamide to the subject at a dose of approximately 60 mg/kg followed by 5 days fludarabine administration at a dose of approximately 25 mg/m2. In some instances, the lymphodepleting treatment regimen optionally or further comprises exposing the subject to total body ionizing irradiation (TBI) at a dose of from about 1 gray to about 80 gray, optionally from about 1 gray to about 20 gray, optionally from about 2 gray to about 15 gray. Murine models had shown that response rates upon TIL therapy improved after prior lymphodepletion by total body irradiation (TBI). The amount of radiation applied varies depending on the type and stage of cancer being treated. Higher doses of radiation are typically administered in the case of solid epithelial tumors where lower doses may be sufficient for non-solid tumors such as lymphomas, and as part of a maintenance protocol from about 0.5 gray to about 4 gray, preferably about 1-2 gray.

Alternatively, In some embodiments, the present disclosure provides therapeutic methods to the treatment of a subject suffering from a disease, disorder or condition, the method comprising the administration to said subject a population engineered human immune cell comprising a genomically-integrated polynucleotide encoding an orthogonal human CD122 (hoCD122) polypeptide in combination with the administration of an IL2 ortholog that is a cognate ligand for the orthogonal CD122 expressed on said orthogonal cells (e.g. orthogonal human immune cells, hoCAR-T cells, hoTILs, hoNK cells) in the absence of lymphodepletion. The methods and compositions of the present disclosure typically obviate the for lymphodepletion of the subject in adoptive cell therapy by both (or either) providing a substantially purified population of engineered cells largely devoid of contamination by non-engineered cells when the foregoing ex vivo method is employed and/or the selective activation and expansion of the orthogonal cells with an IL2 ortholog of the present invention which provide substantially reduced off-target effects of non-specific proliferative agents such as IL2.

In one aspect of the invention, the lymphodepletion currently employed in association with CAR-T therapy may be obviated or reduced by the use of hoCAR-Ts of the present invention. As noted above, the lymphodepletion is commonly employed to enable expansion of the CAR-T cells. However, the lymphodepletion is also associated with major side effects of CAR-T cell therapy. Because the hIL2 ortholog enables the selective activation and expansion of the orthogonal human immune cell (e.g. hoTIL or hoCAR-T) in the mixed population, the cell product administered is substantially enriched for the therapeutically effective orthogonal human immune cell (e.g. hoCD122 TIL or hoCD122 CAR-T) such the need for lymphodepletion prior to administration of the cell product comprising the orthogonal human immune cells is avoided or substantially reduced. The compositions and method of the present invention enable the practice of adoptive cell therapy without or with reduced lymphodepletion prior to administration of the adoptive cell product to the subject.

In some embodiments, the present disclosure provides therapeutic methods to the treatment of a subject suffering from a disease, disorder or condition amenable to treatment with adoptive cell therapy, the method comprising the administration to said subject a population engineered human immune cell comprising a genomically-integrated polynucleotide encoding an orthogonal human CD122 (hoCD122) polypeptide in combination with the administration of an therapeutically effective amount of an hIL2 ortholog that is a cognate ligand for the orthogonal CD122 expressed on said orthogonal cells in the absence of prior lymphodepletion. In one embodiment, the present disclosure provides a method of treating a human subject suffering from a neoplastic disease, disorder or condition with TIL adoptive cell therapy the method comprising administering to said subject a population cells comprising a therapeutically effective amount of hoCD122 TILs in the absence of prior lymphodepletion. In one embodiment, the present disclosure provides a method of treating a human subject suffering from a neoplastic disease, disorder or condition with CAR-T adoptive cell therapy, the method comprising administering to said subject a population cells comprising a therapeutically effective amount of hoCAR-T cells in the absence of prior lymphodepletion.

Treatment of Neoplastic Disease

In one embodiment, the present disclosure provides a method of treating a subject suffering from a hematological neoplastic disease by the administration of a plurality of engineered T cells genomically modified to express the an orthogonal receptor comprising the hoCD122 ECD and a chimeric antigen receptor the extracellular domain of which specifically binds to CD19 and the contemporaneous combination administration of orthogonal IL2 ligand of Formula 1 and the prevention of recurrence of said hematologic neoplastic disease by a maintenance therapy comprising the periodic administration of an orthogonal IL2 ligand of Formula 1.

In one aspect, the hematological cancer is a leukemia or a lymphoma. In one aspect, the term leukemias includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CIVIL), Chronic Lymphoid Leukemia (CLL), B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. In some embodiments, the cancer is multiple myeloma, Hodgkin's lymphoma, non-Hodgkin's lymphoma, or glioblastoma.

In embodiments, the term myeloma inclues e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

Further neoplastic diseases amenable to treatment with the compositions of the present disclosure include atypical and/or non-classical cancers, malignancies, precancerous conditions or proliferative diseases such as a prostate cancer (e.g., castrate-resistant or therapy-resistant prostate cancer, or metastatic prostate cancer), pancreatic cancer, or lung cancer. Non-cancer related conditions amenable to treatment include viral infections and chronic viral infections; e.g., HIV, fungal infections, e.g., C. neoformans; autoimmune disease; e.g. rheumatoid arthritis, system lupus erythematosus (SLE or lupus), pemphigus vulgaris, and Sjogren's syndrome; inflammatory bowel disease, ulcerative colitis; transplant-related allospecific immunity disorders related to mucosal immunity; and unwanted immune responses towards biologies (e.g., Factor VIII) where humoral immunity is important. Additional non-cancer related indications include but are not limited to autoimmune disease, (e.g., lupus), inflammatory disorders (allergy and asthma) and transplantation. In some embodiments, the tumor antigen-expressing cell expresses, or at any time expressed, mRNA encoding the tumor antigen. In an embodiment, the tumor antigen-expressing cell produces the tumor antigen protein (e.g., wild-type or mutant), and the tumor antigen protein may be present at normal levels or reduced levels. In an embodiment, the tumor antigen-expressing cell produced detectable levels of a tumor antigen protein at one point, and subsequently produced substantially no detectable tumor antigen protein. The term “conservative sequence modifications” refers to a

Combination Therapy

The compositions and methods of the present disclosure may be combined with additional therapeutic agents. For example, when the disease, disorder or condition to be treated is a neoplastic disease (e.g. cancer) the methods of the present disclosure may be combined with conventional chemotherapeutic agents or other biological anti-cancer drugs such as checkpoint inhibitors (e.g. PD1 or PDL1 inhibitors) or therapeutic monoclonal antibodies (e.g. Avastin, Herceptin).

As used herein, the term “in combination with” when used in reference to the administration of multiple agents to a subject refers to the administration of a first agent at least one additional (i.e. second, third, fourth, fifth, etc.) agent to a subject. For purposes of the present disclosure, one agent (e.g. an orthogonal cell) is considered to be administered in combination with a second agent (e.g. an ortholog) if the biological effect resulting from the administration of the first agent persists in the subject at the time of administration of the second agent such that the therapeutic effects of the first agent and second agent overlap. For example, an hoCD122 orthogonal CAR-T cells may be administered a single time in a course of therapy while the IL2 orthologs of the present disclosure are typically administered more frequently, e.g. daily, BID, or weekly. However, the administration of the first agent (orthogonal CAR-T cell) t provides a therapeutic effect over an extended time and the administration of the second agent (e.g. an IL2 ortholog) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject.

In certain embodiments, the hoCD122 CAR-T cells and IL2 ortholog are further administered in combination with additional supplementary agent(s) are administered or applied sequentially, e.g., where one agent is administered prior to one or more other agents. In other embodiments, the IL2 ortholog or hoCD122 CAR-T cells and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

Chemotherapeutic Agents:

In some embodiments, the supplementary agent is a chemotherapeutic agent. In some embodiments the supplementary agent is a “cocktail” of multiple chemotherapeutic agents. IN some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A₂, cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C, N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent is\\ one or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the IL2 ortholog is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11:S115), IDO inhibitors such as epacadostat, and oncolytic viruses such as talimogene laherparepvec (T-VEC).

The compositions of the present disclosure may be administered in combination with one or more additional therapeutic agents selected from the group consisting of tyrosine-kinase inhibitors, such as Imatinib mesylate (marketed as Gleevec®, also known as STI-571), Gefitinib (Iressa®, also known as ZD1839), Erlotinib (marketed as Tarceva®), Sorafenib (Nexavar®), Sunitinib (Sutent®), Dasatinib (Sprycel®), Lapatinib (Tykerb®), Nilotinib (Tasigna®), and Bortezomib (Velcade®), Jakafi® (ruxolitinib); Janus kinase inhibitors, such as tofacitinib; ALK inhibitors, such as crizotinib; Bc1-2 inhibitors, such as obatoclax, venclexta, and gossypol; FLT3 inhibitors, such as midostaurin (Rydapt®), IDH inhibitors, such as AG-221, PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel®), everolimus (Afinitor®), Vemurafenib (Zelboraf®), Trametinib (Mekinist), and Dabrafenib (Tafinlar®).

In some embodiments, particularly where the tumor antigen binding domain of the CAR is directed against BCMA, the engineered CAR-T cell is administered in combination with a γ-Secretase Inhibitor (GSI) as described in Pont, et al. (2019) “γ-secretase inhibition increases efficacy of BCMA-specific chimeric antigen receptor T cells in multiple myeloma” Blood https://doi.org/10.1182/blood.2019000050.

Tumor specific monoclonal antibodies that can be administered in combination with an engineered cell may include, without limitation, Rituximab (marketed as MabThera or Rituxan), Alemtuzumab, Panitumumab, Ipilimumab (Yervoy), etc.

Combination with Therapeutic Antibodies

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and tri-specific killer engager (TriKE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Le^{y}, VEGF (e.g. bevacizumab), VEGFR, VEGFR2 (e.g. ramucirumab), PDGFR□ (e.g. olartumumab), EGFR (e.g. cetuximab, panitumumab and necitumumab), ERBB2 (e.g. trastuzumab), ERBB3, MET, IGF1R, EPHA3, TRAIL R1, TRAIL R2, RANKL RAP, tenascin, integrin □V□3, and integrin □4□1.

Examples of antibody therapeutics which are FDA approved and may be used as supplementary agents for use in the treatment of neoplastic disease include but are not limited to [fam]-trastuzumab deruxtecan, enfortumab vedotin, Polatuzumab vedotin Cemiplimab, Moxetumomab pasudotox, mogamuizumab, tildrakizumab, ibalizumab, durvalumab, inotuzumab, ozogamicin, avelumab, atezolizumab, olaratumab, ixekizumab, daratumumab, elotuzumab, necitumumab, dinutuximab, nivolumab. Blinatumomab, pembrolizumab, ramucirumab, siltuximab, obinutuzumab, ado-trastuzumab emtansine, pertuzumab, brentuximab vedotin, ipilimumab, ofatumumab, certolizumab pegol, catumaxomab, panitumumab, bevacizumab, cetuximab, tositumomab-I131, ibritumomab tiuxetan, gemtuzumab, ozogamicin, trastuzumab, infliximab, rituximab, and/or edrecolomab.

In some embodiments, where the antibody is a bispecific antibody targeting a first and second tumor antigen such as HER2 and HER3 (abbreviated HER2×HER3), FAP×DR-5 bispecific antibodies, CEA×CD3 bispecific antibodies, CD20×CD3 bispecific antibodies, EGFR-EDV-miR16 trispecific antibodies, gp100×CD3 bispecific antibodies, Ny-eso×CD3 bispecific antibodies, EGFR×cMet bispecific antibodies, BCMA×CD3 bispecific antibodies, EGFR-EDV bispecific antibodies, CLEC12A×CD3 bispecific antibodies, HER2×HER3 bispecific antibodies, Lgr5×EGFR bispecific antibodies, PD1×CTLA-4 bispecific antibodies, CD123×CD3 bispecific antibodies, gpA33×CD3 bispecific antibodies, B7-H3×CD3 bispecific antibodies, LAG-3×PD1 bispecific antibodies, DLL4×VEGF bispecific antibodies, Cadherin-P×CD3 bispecific antibodies, BCMA×CD3 bispecific antibodies, DLL4×VEGF bispecific antibodies, CD20×CD3 bispecific antibodies, Ang-2×VEGF-A bispecific antibodies, CD20×CD3 bispecific antibodies, CD123×CD3 bispecific antibodies, SSTR2×CD3 bispecific antibodies, PD1×CTLA-4 bispecific antibodies, HER2×HER2 bispecific antibodies, GPC3×CD3 bispecific antibodies, PSMA×CD3 bispecific antibodies, LAG-3×PD-L1 bispecific antibodies, CD38×CD3 bispecific antibodies, HER2×CD3 bispecific antibodies, GD2×CD3 bispecific antibodies, and CD33×CD3 bispecific antibodies.

Such therapeutic antibodies may be further conjugated to one or more chemotherapeutic agents (e.g antibody drug conjugates or ADCs) directly or through a linker, especially acid, base or enzymatically labile linkers.

Combination with Physical Methods:

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising an IL2 ortholog and one or more supplementary agents. In some embodiments, the present disclosure further contemplates the use of an IL2 ortholog in combination with surgery (e.g. tumor resection). In some embodiments, the present disclosure further contemplates the use of an IL2 ortholog in combination with bone marrow transplantation, peripheral blood stem cell transplantation or other types of transplantation therapy.

Combination with Immune Checkpoint Modulators:

In some embodiments, a “supplementary agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

An immune checkpoint whose activation results in inhibition or downregulation of the immune response is referred to herein as a “negative immune checkpoint pathway modulator.” The inhibition of the immune response resulting from the activation of a negative immune checkpoint modulator diminishes the ability of the host immune system to recognize foreign antigen such as a tumor-associated antigen. The term negative immune checkpoint pathway includes, but is not limited to, biological pathways modulated by the binding of PD1 to PDL1, PD1 to PDL2, and CTLA4 to CDCD80/86. Examples of such negative immune checkpoint antagonists include but are not limited to antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152).

In one embodiment, an immune checkpoint pathway the activation of which results in stimulation of the immune response is referred to herein as a “positive immune checkpoint pathway modulator.” The term positive immune checkpoint pathway modulator includes, but is not limited to, biological pathways modulated by the binding of ICOSL to ICOS(CD278), B7-H6 to NKp30, CD155 to CD96, OX40L to OX40, CD70 to CD27, CD40 to CD40L, and GITRL to GITR. Molecules which agonize positive immune checkpoints (such natural or synthetic ligands for a component of the binding pair that stimulates the immune response) are useful to upregulate the immune response. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”).

The immune response mediated by immune checkpoint pathways is not limited to T-cell mediated immune response. For example, the KIR receptors of NK cells modulate the immune response to tumor cells mediated by NK cells. Tumor cells express a molecule called HLA-C, which inhibits the KIR receptors of NK cells leading to a dimunition or the anti-tumor immune response. The administration of an agent that antagonizes the binding of HLA-C to the KIR receptor such an anti-KIR3 mab (e.g. lirilumab, BMS) inhibits the ability of HLA-C to bind the NK cell inhibitory receptor (KIR) thereby restoring the ability of NK cells to detect and attack cancer cells. Thus, the immune response mediated by the binding of HLA-C to the KIR receptor is an example a negative immune checkpoint pathway the inhibition of which results in the activation of a of non-T-cell mediated immune response.

In one embodiment, the immune checkpoint pathway modulator is a negative immune checkpoint pathway inhibitor/antagonist. In another embodiment, immune checkpoint pathway modulator employed in combination with the IL2 ortholog is a positive immune checkpoint pathway agonist. In another embodiment, immune checkpoint pathway modulator employed in combination with the IL2 ortholog is an immune checkpoint pathway antagonist.

The term “negative immune checkpoint pathway inhibitor” refers to an immune checkpoint pathway modulator that interferes with the activation of a negative immune checkpoint pathway resulting in the upregulation or enhancement of the immune response. Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

The term PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline. Aptamer compounds are also described in the literature useful as PD1 pathway inhibitors (Wang, et al. (2018) 145:125-130.).

The term PD1 pathway inhibitors includes peptidyl PD1 pathway inhibitors such as those described in Sasikumar, et al., U.S. Pat. No. 9,422,339 issued Aug. 23, 2016, and Sasilkumar, et al., U.S. Pat. No. 8,907,053 issued Dec. 9, 2014. CA-170 (AUPM-170, Aurigene/Curis) is reportedly an orally bioavailable small molecule targeting the immune checkpoints PDL1 and VISTA. Pottayil Sasikumar, et al. Oral immune checkpoint antagonists targeting PD-L1/VISTA or PD-L1/Tim3 for cancer therapy. [abstract]. In: Proceedings of the 107th Annual Meeting of the American Association for Cancer Research; 2016 Apr. 16-20; New Orleans, LA. Philadelphia (PA): AACR; Cancer Res 2016; 76(14 Suppl): Abstract No. 4861. CA-327 (AUPM-327, Aurigene/Curis) is reportedly an orally available, small molecule that inhibit the immune checkpoints, Programmed Death Ligand-1 (PDL1) and T-cell immunoglobulin and mucin domain containing protein-3 (TIM3).

The term PD1 pathway inhibitors includes small molecule PD1 pathway inhibitors. Examples of small molecule PD1 pathway inhibitors useful in the practice of the present invention are described in the art including Sasikumar, et al., 1,2,4-oxadiazole and thiadiazole compounds as immunomodulators (PCT/IB2016/051266 filed Mar. 7, 2016, published as WO2016142833A1 Sep. 15, 2016) and Sasikumar, et al. 3-substituted-1,2,4-oxadiazole and thiadiazole PCT/IB2016/051343 filed Mar. 9, 2016 and published as WO2016142886A2), BMS-1166 and Chupak L S and Zheng X. Compounds useful as immunomodulators. Bristol-Myers Squibb Co. (2015) WO 2015/034820 A1, EP3041822 B1 granted Aug. 9, 2017; WO2015034820 A1; and Chupak, et al. Compounds useful as immunomodulators. Bristol-Myers Squibb Co. (2015) WO 2015/160641 A2. WO 2015/160641 A2, Chupak, et al. Compounds useful as immunomodulators. Bristol-Myers Squibb Co. Sharpe, et al. Modulators of immunoinhibitory receptor PD-1, and methods of use thereof, WO 2011082400 A2 published Jul. 7, 2011; U.S. Pat. No. 7,488,802 (Wyeth) issued Feb. 10, 2009;

In some embodiments, combination of IL2 orthologs and one or more PD1 immune checkpoint modulators are useful in the treatment of neoplastic conditions for which PD1 pathway inhibitors have demonstrated clinical effect in human beings either through FDA approval for treatment of the disease or the demonstration of clinical efficacy in clinical trials including but not limited to melanoma, non-small cell lung cancer, small cell lung cancer, head and neck cancer, renal cell cancer, bladder cancer, ovarian cancer, uterine endometrial cancer, uterine cervical cancer, uterine sarcoma, gastric cancer, esophageal cancer, DNA mismatch repair deficient colon cancer, DNA mismatch repair deficient endometrial cancer, hepatocellular carcinoma, breast cancer, Merkel cell carcinoma, thyroid cancer, Hodgkins lymphoma, follicular lymphoma, diffuse large B-cell lymphoma, mycosisfungoides, peripheral T-cell lymphoma. In some embodiments, the combination of IL2 orthologs and an PD1 immune checkpoint modulator is useful in the treatment of tumors characterized by high levels of expression of PDL1, where the tumor has a tumor mutational burden, where there are high levels of CD8+ T-cell in the tumor, an immune activation signature associated with IFNγ and the lack of metastatic disease particularly liver metastasis.

In some embodiments, the IL2 ortholog is administered in combination with an antagonist of a negative immune checkpoint pathway that inhibits the binding of CTLA4 to CD28 (“CTLA4 pathway inhibitor”). Examples of CTLA4 pathway inhibitors are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (Medarex, Inc.) issued Oct. 20, 2009)

In some embodiments, the IL2 ortholog is administered in combination with an antagonist of a negative immune checkpoint pathway that inhibits the binding of BTLA to HVEM (“BTLA pathway inhibitor”). A number of approaches targeting the BTLA/HVEM pathway using anti-BTLA antibodies and antagonistic HVEM-Ig have been evaluated, and such approaches have suggested promising utility in a number of diseases, disorders and conditions, including transplantation, infection, tumor, and autoimmune disease (See e.g. Wu, et al., (2012) Int. J. Biol. Sci. 8:1420-30).

In some embodiments, the IL2 ortholog is administered in combination with an antagonist of a negative immune checkpoint pathway that inhibits the ability TIM3 to binding to TIM3-activating ligands (“TIM3 pathway inhibitor”). Examples of TIM3 pathway inhibitors are known in the art and with representative non-limiting examples described in United States Patent Publication No. PCT/US2016/021005 published Sep. 15, 2016; Lifke, et al. United States Patent Publication No. US 20160257749 A1 published Sep. 8, 2016 (F. Hoffman-LaRoche), Karunsky, U.S. Pat. No. 9,631,026 issued Apr. 27, 2017; Karunsky, Sabatos-Peyton, et al. U.S. Pat. No. 8,841,418 isued Sep. 23, 2014; U.S. Pat. No. 9,605,070; Takayanagi, et al., U.S. Pat. No. 8,552,156 issued Oct. 8, 2013.

In some embodiments, the IL2 ortholog is administered in combination with an inhibitor of both LAG3 and PD1 as the blockade of LAG3 and PD1 has been suggested to synergistically reverse anergy among tumor-specific CD8+ T-cells and virus-specific CD8+ T-cells in the setting of chronic infection. IMP321 (ImmuFact) is being evaluated in melanoma, breast cancer, and renal cell carcinoma. See generally Woo et al., (2012) Cancer Res 72:917-27; Goldberg et al., (2011) Curr. Top. Microbiol. Immunol. 344:269-78; Pardoll (2012) Nature Rev. Cancer 12:252-64; Grosso et al., (2007) J. Clin. Invest. 117:3383-392].

In some embodiments, the IL2 ortholog is administered in combination with an A2aR inhibitor. A2aR inhibits T-cell responses by stimulating CD4+ T-cells towards developing into TReg cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the IL2 orthologs for use in the treatment disorders such as cancer and Parkinson's disease.

In some embodiments, the IL2 ortholog is administered in combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g. cancer) in a mammalian subject by the administration of a IL2 ortholog in combination with an agent(s) that modulate at least one immune checkpoint pathway including immune checkpoint pathway modulators that modulate two, three or more immune checkpoint pathways.

In some embodiments the IL2 ortholog is administered in combination with an immune checkpoint modulator that is capable of modulating multiple immune checkpoint pathways. Multiple immune checkpoint pathways may be modulated by the administration of multi-functional molecules which are capable of acting as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of hIL2 orthologs in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the IL2 ortholog may be administered in combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a IL2 ortholog of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

Chemokine and Cytokine Agents as Supplementary Agents:

In some embodiments the IL2 ortholog is administered in combination with additional cytokines including but not limited to IL-7, IL-12, IL-15 and IL-18 including analogs and variants of each thereof.

Activation-Induced Cell Death Inhibitors

In some embodiments the IL2 ortholog is administered in combination with one or more supplementary agents that inhibit Activation-Induced Cell Death (AICD). AICD is a form of programmed cell death resulting from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Examples of agents that inhibit AICD that may be used in combination with the IL2 orthologs described herein include but are not limited to cyclosporin A (Shih, et al., (1989) Nature 339:625-626, IL-16 and analogs (including rhlL-16, Idziorek, et al., (1998) Clinical and Experimental Immunology 112:84-91), TGFb1 (Genesteir, et al., (1999) J Exp Med189(2): 231-239), and vitamin E (Li-Weber, et al., (2002) J Clin Investigation 110(5):681-690).

### EXAMPLES

The following examples are offered to illustrate, but not to limit the claimed invention.

### Example 1: CRISPR Knock-In Strategy for Human orthoIL2Rb

The following strategy is used to engineer a recombinant human immune T cell to express an hoCD122 species comprising the mutations H133D Y134F (numbered in accordance with wild-type hCD122) into the endogenous hCD122 genomic locus of a T-cell using the CRISPR Cas9 technology which is well known to those of skill in the art. Recombinant Cas9, IL2Rb targeting sgRNA, and a single-stranded DNA homology donor repair (HDR) template encoding the orthogonal mutations are electroporated into primary T cells or T cell clones. Cells are then anti-CD3/CD28-stimulated and grown in T-cell growth medium T cell growth media (e.g. OpTmizer, TexMACS, RPMI) containing orthogonal IL-2 ligand to select and enrich for cells that incorporate the hoCD122 mutation. Transduction of CARs can be performed 48 h post-stimulation. Genomic editing efficiency is performed by restriction fragment length polymorphism (RFLP) assay and/or DNA sequencing of the orthogonal mutation locus.

### Example 2. sgRNA Design

Three 20 bp sgRNAs (Table 3) targeting regions within 30 bp of the orthoIL2Rb mutation site are selected based upon their specificity and efficiency scores (>60 on scale of 1-100). sgRNAs with chemical modifications to reduce off target effects and overall improved editing efficiency will be ordered from Synthego (world wide web at: synthego.com/help/grnas-chemical-modifications). See Table 3 below.

### Example 3. Ortho Mutant HDR Template Design

The H133 codon (CAC) is changed to D (GAT or GAC) and the Y134 codon (TAC) is changed to F (TTT or TTC). To start, CAC TAC is changed to GAC TTC. The template encodes the H133D and Y134F changes, a silent mutation within the sgRNA PAM site (to prevent possible recutting), and another silent mutation to create a novel restriction enzyme site (NheI) within the orthoIL2Rb loci for the RFLP assay. The template has symmetrical 75-bp homology arms flanking the orthoIL2Rb mutation site. These are generated as single stranded oligonucleotides. Optionally, HDR templates with asymmetric homology arms 36-bp 3′ of the PAM and 91-bp 5′ of the PAM can be used.

### Example 4. Introduction of Reagents into the Cells

While there are numerous approaches to introduce Cas9 and the sgRNAs into cells (e.g. lentiviral transduction, plasmid-based transfections), electroporation of recombinant Cas9, synthetic sgRNA, and the HDR template is a very efficient and GMP suitable approach for cell therapy.

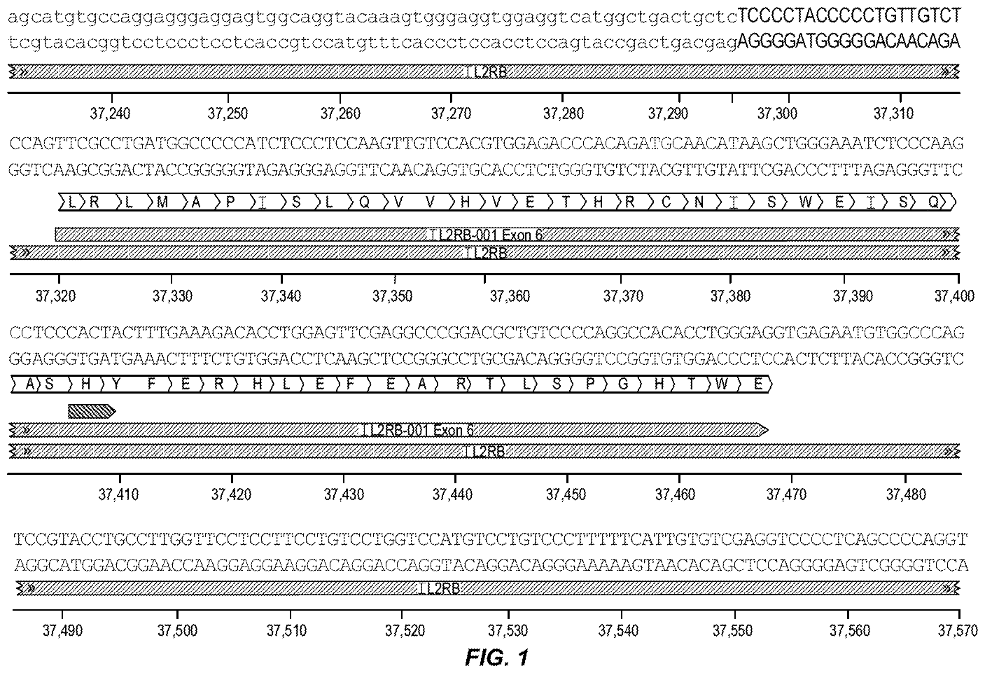

FIG. 1 depicts a portion of the CD122 coding sequence with positions for mutation of H133 and D134.

TABLE 3
(Table 3 discloses SEQ ID NOS 18-32, in order of column)

| | | | | | | | | | HDR template | |
|---|---|---|---|---|---|---|---|---|---|---|
| | | | | | | | HDR template | | sequence with | |
| | | | | | | | sequence | | XhoI site | |
| | | | | | | | | reverse | | reverse |
| | Posi- | | Se- | | Specificity | Efficiency | | comple- | | comple- |
| sgRNA | tion | Strand | quence | PAM | Score | Score | | ment | | ment |
| sghIL2Rb_ | 37407 | −1 | AGTAGTG | AGG | 67.7613225 | 67.52337428 | GCCCCCA | GCCACAT | GCCCCCA | GCCACAT |
| ortho_ | | | GGGTGTC | | | | TCTCCCT | TCTCACC | TCTCCCT | TCTCACC |
| 1 | | | TTTCAA | | | | CCAAGTT | TCCCAGG | CCAAGTT | TCCCAGG |
| | | | | | | | GTCCACG | TGTGGCC | GTCCACG | TGTGGCC |
| | | | | | | | TGGAGAC | TGGGGAC | TGGAGAC | TGGGGAC |
| | | | | | | | CCACAGA | AGCGTCC | CCACAGA | AGCGTCC |
| | | | | | | | TGCAACA | GGGCCTC | TGCAACA | GGGCCTC |
| | | | | | | | TAAGCTG | GAACTCC | TAAGCTG | GAACTCG |
| | | | | | | | GGAAATC | AGGTGTC | GGAAATC | AGGTGTC |
| | | | | | | | TCCCAAG | TTTCAAA | TCCCAAG | TTTCAAA |
| | | | | | | | CATCCgA | GaAGTcG | CATCCGA | GAAGTCG |
| | | | | | | | CTtCTTT | GATGCTT | CTTCTTT | GATGCTT |
| | | | | | | | GAAAGAC | GGGAGAT | GAAAGAC | GGGAGAT |
| | | | | | | | ACCTGGA | TTCCCAG | ACCTCGA | TTCCCAG |
| | | | | | | | GTTCGAG | CTTATGT | GTTCGAG | CTTATGT |
| | | | | | | | GCCCGGA | TGCATCT | GCCCGGA | TGCATCT |
| | | | | | | | CGCTGTC | GTGGGTC | CGCTGTC | GTGGGTC |
| | | | | | | | CCCAGGC | TCCACGT | CCCAGGC | TCCACGT |
| | | | | | | | CACACCT | GGACAAC | CACACCT | GGACAAC |
| | | | | | | | GGGAGGT | TTGGAGG | GGGAGGT | TTGGAGG |
| | | | | | | | GAGAATG | GAGATGG | GAGAATG | GAGATGG |
| | | | | | | | TGGC | GGGC | TGGC | GGGC |
| sghIL2Rb_ | 37383 | 1 | CCACAGA | GGG | 70.1530894 | 66.30011904 | GCCCCCA | GCCACAT | GCCCCCA | GCCACAT |
| ortho_ | | | TGCA | | | | TCTCCCT | TCTCACC | TCTCCCT | TCTCACC |
| 2 | | | | | | | CCAAGTT | TCCCAGG | CCAAGTT | TCCCAGG |
| | | | | | | | GTCCACG | TGTGGCC | GTCCACG | TGTGGCC |
| | | | | | | | TGGAGAC | TGGGGAC | TGGAGAC | TGGGGAC |
| | | | | | | | CCACAGG | AGCGTCC | CCACAGG | AGCGTCC |
| | | | | | | | TGCAACA | GGGCCTC | TGCAACA | GGGCCTC |
| | | | | | | | TATCCTG | GAACTCC | TATCCTG | GAACTCG |
| | | | | | | | GGAAATC | AGGTGTC | GGAAATC | AGGTGTC |
| | | | | | | | TCCCAAG | TTTCAAA | TCCCAAG | TTTCAAA |
| | | | | | | | CCTCCgA | GaAGTcG | CCTCCGA | GAAGTCG |
| | | | | | | | CTtCTTT | GAGGCTT | CTTCTTT | GAGGCTT |
| | | | | | | | GAAAGAC | GGGAGAT | GAAAGAC | GGGAGAT |
| | | | | | | | ACCTGGA | TTCCCAG | ACCTCGA | TTCCCAG |
| | | | | | | | GTTCGAG | GATATGT | GTTCGAG | GATATGT |
| | | | | | | | GCCCGGA | TGCACCT | GCCCGGA | TGCACCT |
| | | | | | | | CGCTGTC | GTGGGTC | CGCTGTC | GTGGGTC |
| | | | | | | | CCCAGGC | | CCCAGGC | TCCACGT |
| | | | | | | | CACACCT | | CACACCT | GGA |
| | | | | | | | GGGAG | | GGGAGGT | |
| | | | | | | | | | GAGAA | |
| sghIL2Rb_ | 37410 | −1 | CAGGTGT | GGG | 64.9225092 | 60.63085149 | GCCCCCA | GCCACAT | GCCCCCA | GCCACAT |
| ortho_ | | | CTTT [symbol] | | | | TCTCCCT | TCTCACC | TCTCCCT | TCTCACC |
| 3 | | | | | | | CCAAGTT | TCCCAGG | CCAAGTT | TCCCAGG |
| | | | | | | | GTCCACG | TGTGGCC | GTCCACG | TGTGGCC |
| | | | | | | | TGGAGAC | TGGGGAC | TGGAGAC | TGGGGAC |
| | | | | | | | CCACAGA | AGCGTCC | CCACAGA | AGCGTCC |
| | | | | | | | TGCAACA | GGGCCTC | TGCAACA | GGGCCTC |
| | | | | | | | TAAGCTG | GAACTCC | TAAGCTG | GAACTCG |
| | | | | | | | GGAAATC | AGGTGTC | GGAAATC | AGGTGTC |
| | | | | | | | TCCCAAG | TTTCAAA | TCCCAAG | TTTCAAA |
| | | | | | | | CCAGCgA | GaAGTcG | CCAGCGA | GAAGTCG |
| | | | | | | | CTtCTTT | CTGGCTT | CTTCTTT | CTGGCTT |
| | | | | | | | GAAAGAC | GGGAGAT | GAAAGAC | GGGAGAT |
| | | | | | | | ACCTGGA | TTCCCAG | ACCTCGA | TTCCCAG |
| | | | | | | | GTTCGAG | CTTATGT | GTTCGAG | CTTATGT |
| | | | | | | | GCCCGGA | | GCCCGGA | TGCATCT |
| | | | | | | | CGCTGTC | | CGCTGTC | GT |
| | | | | | | | CC | | CCCAGGC | |

[symbol]

indicates data missing or illegible when filed

All publications, patents and patent applications cited in this specification are herein incorporated by reference as if each individual publication or patent application were specifically and individually indicated to be incorporated by reference. Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. A human lymphocyte or myeloid cell comprising a polynucleotide encoding an engineered hoCD122, wherein the lymphocyte or myeloid cell does not express native human CD122.

2. The human lymphocyte or myeloid cell of claim 1, wherein the polynucleotide encoding an engineered hoCD122 is inserted in place of the endogenous human CD locus.

3. The human lymphocyte or myeloid cell of claim 1, wherein the hoCD122 is modified at one or more residues selected from R41, R42, Q70, K71, T73, T74, V75, S132, H133, Y134, F135, E136, and Q214 relative to native human CD122.

4. The human lymphocyte or myeloid cell of claim 1, wherein the hoCD122 is modified at H133 and Y134 relative to native human CD122.

5. The human lymphocyte or myeloid cell of claim 3, wherein the engineered hoCD122 comprises an amino acid sequence at least 95% identical to SEQ ID NO:1.

6. The human lymphocyte or myeloid cell of claim 5, wherein the engineered hoCD122 comprises SEQ ID NO:1 modified to have H133D and Y134F substitutions.

7. The human lymphocyte or myeloid cell of claim 1, wherein the lymphocyte further expresses a chimeric antigen receptor (CAR).

8. The human lymphocyte or myeloid cell of claim 7, wherein the CAR is selected from the group consisting of a CD19 chimeric antigen receptor (CAR), a B-Cell Maturation Antigen (BCMA) CAR, a CD123 CAR, a CD20 CAR, a CD22 CAR, a CD30 CAR, a CD70 CAR, a Lewis Y CAR, a GD3 CAR, a GD3 CAR, a mesothelin CAR, a ROR CAR, a CD44 CAR, a CD171 CAR, a EGP2 CAR, a EphA2 CAR, a ErbB2 CAR, a ErbB3/4 CAR, a FAP CAR, a FAR CAR, a IL11Ra CAR, a PSCA CAR, a PSMA CAR and a NCAM CAR.

9. The human lymphocyte or myeloid cell of claim 1, wherein the lymphocyte or myeloid cell is deleted for one or more of T cell receptor alpha (TCRA), T cell receptor beta (TCRB), PD-1, cytotoxic T-lymphocyte-associated protein 4 (CTLA4) or beta2 microglobulin (B2M).

10. The human lymphocyte or myeloid cell of claim 1, wherein the cell is a T-cell.

11. A method of expanding the human lymphocyte or myeloid cell of claim 1, the method comprising,
contacting the human lymphocyte or myeloid cell with an orthogonal human IL-2, wherein contact of the orthogonal human IL-2 to the engineered hoCD122 results of expansion of the human lymphocyte or myeloid cell.

12. A method of making a human lymphocyte or myeloid cell of claim 1 comprising a polynucleotide encoding an engineered hoCD122 in place of the endogenous human CD122 locus, the method comprising,
providing a human lymphocyte or myeloid cell; and
introducing the polynucleotide in the endogenous human CD122 locus of the lymphocyte or myeloid cell, thereby making a human lymphocyte comprising a polynucleotide encoding an engineered hoCD122 in place of the endogenous human CD122 locus.

13-32. (canceled)

33. A nucleic acid comprising a homology directed repair (HDR) template comprising a polynucleotide encoding an engineered hoCD122 comprising homology arms for insertion into an endogenous human CD122 locus.

34. The nucleic acid of claim 33, wherein the polynucleotide encoding an engineered hoCD122 comprises one or more mutation relative to the endogenous human CD122 locus such that one or more CRISPR protospacer adjacent motif (PAM) site is eliminated, optionally wherein the mutation results in a silent codon change.

35. The nucleic acid of claim 33, wherein the HDR template further comprises a CAR-encoding polynucleotide.

36. The nucleic acid of claim 35, wherein the engineered hoCD122 and the CAR are encoded as a single fusion protein separated by a self-cleaving peptide sequence.

37. A composition comprising the nucleic acid of claim 33 and (i) a nuclease targeted to an endogenous human CD122 locus, (ii) a polynucleotide encoding the nuclease targeted to an endogenous human CD122 locus or (iii) a viral vector targeted to an endogenous human CD122 locus.

38-40. (canceled)
